(19) United States
(12) Patent Application Publication
(10) Pub. No.: US 2010/0292233 A1
(43) Pub. Date: Nov. 18, 2010

(54) DIHYDRO-1H-PYRROLO[1,2-A]INDOL-1-YL CARBOXYLIC ACID DERIVATIVES WHICH ACT AS S1P1 AGONISTS

(75) Inventors: Robert M. Jones, San Diego, CA (US); Daniel J. Buzard, San Diego, CA (US); Andrew M. Kawasaki, San Diego, CA (US); Luis A. Lopez, San Diego, CA (US); Jeanne V. Moody, San Diego, CA (US); Lars Thoresen, San Diego, CA (US); Brett Ullman, San Diego, CA (US)

Correspondence Address: FISH & RICHARDSON P.C., P.O. BOX 1022, MINNEAPOLIS, MN, 55440-1022, US

(73) Assignee: ARENA PHARMACEUTICALS, INC., San Diego, CA (US)

(21) Appl. No.: 12/863,637

(22) PCT Filed: Jan. 22, 2009
(86) PCT No.: PCT/US09/00399

§ 371 Date: Jul. 20, 2010

Related U.S. Application Data
(60) Provisional application No. 61/062,368, filed on Jan. 25, 2008.

(51) Int. Cl. A61K 31/4245; C07D 487/04; A61K 31/4439; A61K 31/497; A61K 31/5377; A61P 37/00; A61P 35/00; A61P 29/00; A61P 17/06; A61P 1/04; A61P 3/10; A61P 9/10; A61P 17/10; A61P 31/00; A61P 31/12

(52) U.S. Cl. 514/233.2; 548/131; 546/269.4; 544/405; 544/131; 514/364; 514/339; 514/255.05

(57) ABSTRACT

The present invention relates to certain (1,2,4-oxadiazol-3-yl)-2,3-dihydro-1H-pyrrolo[1,2-a]indol-1-yl carboxylic acid derivatives of Formula (Ia) and pharmaceutically acceptable salts thereof, which exhibit useful pharmacological properties, for example, as agonists of the S1P1 receptor. Also provided by the present invention are pharmaceutical compositions containing compounds of the invention, and methods of using the compounds and compositions of the invention in the treatment of S1P1 associated disorders, for example, psoriasis, rheumatoid arthritis, Crohn's disease, transplant rejection, multiple sclerosis, systemic lupus erythematosus, ulcerative colitis, type I diabetes, sepsis, myocardial infarction, ischemic stroke, acne, microbial infections or diseases and viral infections or diseases.

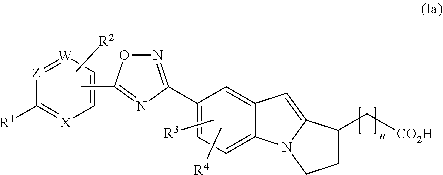

The present invention relates to certain (1,2,4-oxadiazol-3-yl)-2,3-dihydro-1H-pyrrolo[1,2-a]indol-1-yl carboxylic acid derivatives of Formula (Ia) and pharmaceutically acceptable salts thereof, which exhibit useful pharmacological properties, for example, as agonists of the S1P1 receptor. Also provided by the present invention are pharmaceutical compositions containing compounds of the invention, and methods of using the compounds and compositions of the invention in the treatment of S1P1 associated disorders, for example, psoriasis, rheumatoid arthritis, Crohn's disease, transplant rejection, multiple sclerosis, systemic lupus erythematosus, ulcerative colitis, type I diabetes, sepsis, myocardial infarction, ischemic stroke, acne, microbial infections or diseases and viral infections or diseases.

### BACKGROUND OF THE INVENTION

The present invention relates to compounds that are S1P1 receptor agonists having at least immunosuppressive, anti-inflammatory and/or hemostatic activities, e.g. by virtue of modulating leukocyte trafficking, sequestering lymphocytes in secondary lymphoid tissues, and/or enhancing vascular integrity.

The present application is in part focused on addressing an unmet need for immunosuppressive agents such as may be orally available which have therapeutic efficacy for at least autoimmune diseases and disorders, inflammatory diseases and disorders (e.g., acute and chronic inflammatory conditions), transplant rejection, cancer, and/or conditions that have an underlying defect in vascular integrity or that are associated with angiogenesis such as may be pathologic (e.g., as may occur in inflammation, tumor development and atherosclerosis) with fewer side effects such as the impairment of immune responses to systemic infection.

The sphingosine-1-phosphate (S1P) receptors 1-5 constitute a family of G protein-coupled receptors with a seven-transmembrane domain. These receptors, referred to as S1P1 to S1P5 (formerly termed endothelial differentiation gene (EDG) receptor-1, -5, -3, -6 and -8, respectively; Chun et al., Pharmacological Reviews, 54:265-269, 2002), are activated via binding by sphingosine-1-phosphate, which is produced by the sphingosine kinase-catalyzed phosphorylation of sphingosine. S1P1, S1P4 and S1P5 receptors activate Gi but not Gq, whereas S1P2 and S1P3 receptors activate both Gi and Gq. The S1P3 receptor, but not the S1P1 receptor responds to an agonist with an increase in intracellular calcium.

S1P receptor agonists having agonist activity on the S1P1 receptor have been shown to rapidly and reversibly induce lymphopenia (also referred to as peripheral lymphocyte lowering (PLL); Hale et al., Bioorg. Med. Chem. Lett., 14:3351-3355, 2004). This is attended by clinically useful immunosuppression by virtue of sequestering T- and B-cells in secondary lymphoid tissue (lymph nodes and Peyer's patches) and thus apart from sites of inflammation and organ grafts (Rosen et al., Immunol. Rev., 195:160-177, 2003; Schwab et al., Nature Immunol., 8:1295-1301, 2007). This lymphocyte sequestration, for example in lymph nodes, is thought to be a consequence of concurrent agonist-driven functional antagonism of the S1P1 receptor on T-cells (whereby the ability of S1P to mobilize T-cell egress from lymph nodes is reduced) and persistent agonism of the S1P1 receptor on lymph node endothelium (such that barrier function opposing transmigration of lymphocytes is increased) (Matloubian et al., Nature, 427:355-360, 2004; Baumruker et al., Expert Opin. Investig. Drugs, 16:283-289, 2007). It has been reported that agonism of the S1P1 receptor alone is sufficient to achieve lymphocyte sequestration (Sarna et al., J Biol Chem., 279:13839-13848, 2004) and that this occurs without impairment of immune responses to systemic infection (Brinkmann et al., Transplantation, 72:764-769, 2001; Brinkmann et al., Transplant Proc., 33:530-531, 2001).

That agonism of the endothelial S1P1 receptor has a broader role in promoting vascular integrity is supported by work implicating the S1P1 receptor in capillary integrity in mouse skin and lung (Sarna et al., Nat Chem Biol., 2:434-441, 2006). Vascular integrity can be compromised by inflammatory processes, for example as may derive from sepsis, major trauma and surgery so as to lead to acute lung injury or respiratory distress syndrome (Johan Groeneveld, Vascul. Pharmacol., 39:247-256, 2003).

An exemplary S1P receptor agonist having agonist activity on the S1P1 receptor is FTY720 (fingolimod), an immunosuppressive agent currently in clinical trials (Martini et al., Expert Opin. Investig. Drugs, 16:505-518, 2007). FTY720 acts as a prodrug which is phosphorylated in vivo; the phosphorylated derivative is an agonist for S1P1, S1P3, S1P4 and S1P5 receptors (but not the S1P2 receptor) (Chiba, Pharmacology & Therapeutics, 108:308-319, 2005). FTY720 has been shown to rapidly and reversibly induce lymphopenia (also referred to as peripheral lymphocyte lowering (PLL); Hale et al., Bioorg. Med. Chem. Lett., 14:3351-3355, 2004). This is attended by clinically useful immunosuppression by virtue of sequestering T- and B-cells in secondary lymphoid tissue (lymph nodes and Peyer's patches) and thus apart from sites of inflammation and organ grafts (Rosen et al., Immunol. Rev., 195:160-177, 2003; Schwab et al., Nature Immunol., 8:1295-1301, 2007).

In clinical trials, FTY720 elicited an adverse event (i.e., transient asymptomatic bradycardia) due to its agonism of the S1P3 receptor (Budde et al., J. Am. Soc. Nephrol., 13:1073-1083, 2002; Sanna et al., J. Biol. Chem., 279:13839-13848, 2004; Ogawa et al., BBRC, 361:621-628, 2007).

FTY720 has been reported to have therapeutic efficacy in at least: a rat model for autoimmune myocarditis and a mouse model for acute viral myocarditis (Kiyabayashi et al., J. Cardiovasc. Pharmacol., 35:410-416, 2000; Miyamoto et al., J. Am. Coll. Cardiol., 37:1713-1718, 2001); mouse models for inflammatory bowel disease including colitis (Mizushima et al., Inflamm. Bowel Dis., 10:182-192, 2004; Deguchi et al., Oncology Reports, 16:699-703, 2006; Fujii et al., Am. J. Physiol. Gastrointest. Liver Physiol., 291:G267-G274, 2006; Daniel et al., J. Immunol., 178:2458-2468, 2007); a rat model for progressive mesangioproliferative glomerulonephritis (Martini et al., Am. J. Physiol. Renal Physiol., 292:F1761-F1770, 2007); a mouse model for asthma, suggested to be primarily through the S1P1 receptor on the basis of work using the the S1P1 receptor agonist SEW2871 (Idzko et al, J. Clin. Invest., 116:2935-2944, 2006); a mouse model for airway inflammation and induction of bronchial hyperresponsiveness (Sawicka et al., J. Immunol., 171;6206-6214, 2003); a mouse model for atopic dermatitis (Kohno et al., Biol. Pharm. Bull., 27:1392-1396, 2004); a mouse model for ischemia-reperfusion injury (Kaudel et al., Transplant. Proc, 39:499-502, 2007); a mouse model for systemic lupus erythematosus (SLE) (Okazaki et al., J. Rheumatol., 29:707-716, 2002; Herzinger et al, Am. J. Clin. Dermatol., 8:329-336, 2007); rat models for rheumatoid arthritis (Matsuura et al., Int. J. Immunopharmacol., 22:323-331, 2000; Matsuura et al., Inflamm. Res., 49:404-410, 2000); a rat model for autoimmune uveitis (Kurose et al., Exp. Eye Res., 70:7-15, 2000); mouse models for type I diabetes (Fu et al, Transplantation, 73:1425-1430, 2002; Maki et al., Transplantation, 74:1684-1686, 2002; Yang et al., Clinical Immunology, 107:30-35, 2003; Maki et al., Transplantation, 79:1051-1055, 2005); mouse models for atherosclerosis (Nofer et al., Circulation, 115:501-508, 2007; Keul et al., Arterioscler. Thromb. Vase. Biol., 27:607-613, 2007); a rat model for brain inflammatory reaction following traumatic brain injury (TBI) (Zhang et al., J. Cell. Mol. Med., 11:307-314, 2007); and mouse models for graft coronary artery disease and graft-versus-host disease (GVHD) (Hwang et al., Circulation, 100:1322-1329, 1999; Taylor et al., Blood, 110:3480-3488, 2007). In vitro results suggest that FTY720 may have therapeutic efficacy for β-amyloid-related inflammatory diseases including Alzheimer's disease (Kaneider et al., FASEB J., 18:309-311, 2004). KRP-203, an S1P receptor agonist having agonist activity on the S1P1 receptor, has been reported to have therapeutic efficacy in a rat model for autoimmune myocarditis (Ogawa et al., BBRC, 361:621-628, 2007). Using the S1P1 receptor agonist SEW2871, it has been shown that agonism of the endothelial S1P1 receptor prevents proinflammatory monocyte/endothelial interactions in type I diabetic vascular endothelium (Whetzel et al., Circ. Res., 99:731-739, 2006) and protects the vasculature against TNFα-mediated monocyte/endothelial interactions (Bolick et al., Arterioscler. Thromb. Vasc. Biol., 25:976-981, 2005).

Additionally, FTY720 has been reported to have therapeutic efficacy in experimental autoimmune encephalomyelitis (EAE) in rats and mice, a model for human multiple sclerosis (Brinkmann et al., J. Biol. Chem., 277:21453-21457, 2002; Fujino et al., J. Pharmacol. Exp. Ther., 305:70-77, 2003; Webb et al., J. Neuroimmunol., 153:108-121, 2004; Rausch et al., J. Magn. Reson. Imaging, 20:16-24, 2004; Kataoka et al., Cellular & Molecular Immunology, 2:439-448, 2005; Brinkmann et al., Pharmacology & Therapeutics, 115:84-105, 2007; Baumruker et al., Expert Opin. Investig. Drugs, 16:283-289, 2007; Balatoni et al., Brain Research Bulletin, 74:307-316, 2007). Furthermore, FTY720 has been found to have therapeutic efficacy for multiple sclerosis in clinical trials. In Phase II clinical trials for relapsing-remitting multiple sclerosis, FTY720 was found to reduce the number of lesions detected by magnetic resonance imaging (MRI) and clinical disease activity in patients with multiple sclerosis (Kappos et al., N. Engl. J. Med., 355:1124-1140, 2006; Martini et al., Expert Opin. Investig. Drugs, 16:505-518, 2007; Zhang et al., Mini-Reviews in Medicinal Chemistry, 7:845-850, 2007; Brinkmann, Pharmacology & Therapeutics, 115:84-105, 2007). FTY720 is currently in Phase III studies of remitting-relapsing multiple sclerosis (Brinkmann, Pharmacology & Therapeutics, 115:84-105, 2007; Baumruker et al., Expert. Opin. Investig. Drugs, 16:283-289, 2007; Dev et al., Pharmacology and Therapeutics, 117:77-93, 2008).

Recently, FTY720 has been reported to have anti-viral activity. Specific data has been presented in the lymphocytic choriomeningitis virus (LCMV) mouse model, wherein the mice were infected with either the Armstrong or the clone 13 strain of LCMV (Premenko-Lanier et al., Nature, 454, 894, 2008).

FTY720 has been reported to impair migration of dendritic cells infected with Francisella tularensis to the mediastinal lymph node, thereby reducing the bacterial colonization of it. Francisella tularensis is associated with tularemia, ulceroglandular infection, respiratory infection and a typhoidal disease (E. Bar-Haim et al, PLoS Pathogens, 4(11): e1000211.doi:10.1371/journal.ppat.1000211, 2008).

It has also been recently reported that a short-term high dose of FTY720 rapidly reduced ocular infiltrates in experimental autoimmune uveoretinitis. When given in the early stages of ocular inflammation, FTY720 rapidly prevented retinal damage. It was reported to not only prevent infiltration of target organs, but also reduce existing infiltration (Raveney et al., Arch. Ophthalmol. 126(10), 1390, 2008).

Agonism of the S1P1 receptor has been implicated in enhancement of survival of oligodendrocyte progenitor cells. Survival of oligodendrocyte progenitor cells is a required component of the remyelination process. Remyelination of multiple sclerosis lesions is considered to promote recovery from clinical relapses. (Miron et al., Ann. Neurol., 63:61-71, 2008; Coelho et al., J. Pharmacol. Exp. Ther., 323:626-635, 2007; Dev et al., Pharmacology and Therapeutics, 117:77-93, 2008). It also has been shown that the S1P1 receptor plays a role in platelet-derived growth factor (PDGF)-induced oligodendrocyte progenitor cell mitogenesis (Jung et al., Glia, 55:1656-1667, 2007).

Agonism of the S1P1 receptor has also been reported to mediate migration of neural stem cells toward injured areas of the central nervous system (CNS), including in a rat model of spinal cord injury (Kimura et al., Stem Cells, 25:115-124, 2007).

Agonism of the S1P1 receptor has been implicated in the inhibition of keratinocyte proliferation (Sauer et al., J. Biol. Chem., 279:38471-38479, 2004), consistent with reports that S1P inhibits keratinocyte proliferation (Kim et al., Cell Signal, 16:89-95, 2004). The hyperproliferation of keratinocytes at the entrance to the hair follicle, which can then become blocked, and an associated inflammation are significant pathogenetic factors of acne (Koreck et al., Dermatology, 206:96-105, 2003; Webster, Cutis, 76:4-7, 2005).

FTY720 has been reported to have therapeutic efficacy in inhibiting pathologic angiogenesis, such as that as may occur in tumor development. Inhibition of angiogenesis by FTY720 is thought to involve agonism of the S1P1 receptor (Oo et al., J. Biol. Chem., 282;9082-9089, 2007; Schmid et al., J. Cell Biochem., 101:259-270, 2007). FTY720 has been reported to have therapeutic efficacy for inhibiting primary and metastatic tumor growth in a mouse model of melanoma (LaMontagne et al., Cancer Res., 66:221-231, 2006). FTY720 has been reported to have therapeutic efficacy in a mouse model for metastatic hepatocellular carcinoma (Lee et al., Clin. Cancer Res., 11:84588466, 2005).

It has been reported that oral administration of FTY720 to mice potently blocked VEGF-induced vascular permeability, an important process associated with angiogenesis, inflammation, and pathological conditions such as sepsis, hypoxia, and solid tumor growth (T Sanchez et al, J. Biol. Chem., 278(47), 47281-47290, 2003).

Cyclosporin A and FK506 (calcineurin inhibitors) are drugs used to prevent rejection of transplanted organs. Although they are effective in delaying or suppressing transplant rejection, classical immunosuppressants such as cyclosporin A and FK506 are known to cause several undesirable side effects including nephrotoxicity, neurotoxicity, β-cell toxicity and gastrointestinal discomfort. There is an unmet need in organ transplantation for an immunosuppressant without these side effects which is effective as a monotherapy or in combination with a classical immunosuppressant for inhibiting migration of, e.g., alloantigen-reactive T-cells to the grafted tissue, thereby prolonging graft survival.

FTY720 has been shown to have therapeutic efficacy in transplant rejection both as a monotherapy and in synergistic combination with a classical immunosuppressant, including cyclosporin A, FK506 and RAD (an mTOR inhibitor). It has been shown that, unlike the classical immunosuppressants cyclosporin A, FK506 and RAD, FTY720 has efficacy for prolonging graft survival without inducing general immunosuppression, and this difference in drug action is believed to be relevant to the synergism observed for the combination (Brinkmann et al., Transplant Proc., 33:530-531, 2001; Brinkmann et al., Transplantation, 72:764-769, 2001).

Agonism of the S1P1 receptor has been reported to have therapeutic efficacy for prolonging allograft survival in mouse and rat skin allograft models (Lima et al., Transplant Proc., 36:1015-1017, 2004; Yan et al., Bioorg. & Med. Chem. Lett., 16:3679-3683, 2006). FTY720 has been reported to have therapeutic efficacy for prolonging allograft survival in a rat cardiac allograft model (Suzuki et al., Transpl. Immunol., 4:252-255, 1996). FTY720 has been reported to act synergistically with cyclosporin A to prolong rat skin allograft survival (Yanagawa et al., J. Immunol., 160:5493-5499, 1998), to act synergistically with cyclosporin A and with FK506 to prolong rat cardiac allograft survival, and to act synergistically with cyclosporin A to prolong canine renal allograft survival and monkey renal allograft survival (Chiba et al., Cell Mol. Biol., 3:11-19, 2006). KRP-203, an S1P receptor agonist has been reported to have therapeutic efficacy for prolonging allograft survival in a rat skin allograft model and both as monotherapy and in synergistic combination with cyclosporin A in a rat cardiac allograft model (Shimizu et al., Circulation, 111:222-229, 2005). KRP-203 also has been reported to have therapeutic efficacy in combination with mycophenolate mofetil (MMF; a prodrug for which the active metabolite is mycophenolic acid, an inhibitor of purine biosynthesis) for prolonging allograft survival both in a rat renal allograft model and in a rat cardiac allograft model (Suzuki et al., J. Heart Lung Transplant, 25:302-209, 2006; Fujishiro et al., J. Heart Lung Transplant, 25:825-833, 2006). It has been reported that an agonist of the S1P1 receptor, AUY954, in combination with a subtherapeutic dose of RAD001 (Certican/Everolimus, an mTOR inhibitor) can prolong rat cardiac allograft survival (Pan et al., Chemistry & Biology, 13:1227-1234, 2006). In a rat small bowel allograft model, FTY720 has been reported to act synergistically with cyclosporin A to prolong small bowel allograft survival (Sakagawa et al., Transpl. Immunol., 13:161-168, 2004). FTY720 has been reported to have therapeutic efficacy in a mouse islet graft model (Fu et al., Transplantation, 73:1425-1430, 2002; Liu et al., Microsurgery, 27:300-304; 2007) and in a study using human islet cells to evidence no detrimental effects on human islet function (Truong et al., American Journal of Transplantation, 7:2031-2038, 2007).

FTY720 has been reported to reduce the nociceptive behavior in the spared nerve injury model for neuropathic pain which does not depend on prostaglandin synthesis (O. Costu et al, Journal of Cellular and Molecular Medicine 12(3), 995-1004, 2008).

FTY720 has been reported to impair initiation of murine contact hypersensitivity (CHS). Adoptive transfer of immunized lymph node cells from mice treated with FTY720 during the sensitization phase was virtually incapable of inducing CHS response in recipients (D. Nakashima et al., J. Investigative Dermatology (128(12), 2833-2841, 2008).

It has been reported that prophylactic oral administration of FTY720 (1 mg/kg, three times a week), completely prevented the development of experimental autoimmune myasthenia gravis (EAMG) in C57BL/6 mice (T. Kohono et al, Biological & Pharmaceutical Bulletin, 28(4), 736-739, 2005).

In one embodiment, the present invention encompasses compounds which are agonists of the S1P1 receptor having selectivity over the S1P3 receptor. The S1P3 receptor, and not the S1P1 receptor, has been directly implicated in bradycardia (Sanna et al., J. Biol. Chem., 279:13839-13848, 2004). An S1P1 receptor agonist selective over at least the S1P3 receptor has advantages over current therapies by virtue of an enhanced therapeutic window, allowing better tolerability with higher dosing and thus improving efficacy as therapy. The present invention encompasses compounds which are agonists of the S1P1 receptor and which exhibit no or substantially no activity for bradycardia.

S1P1 receptor agonists are useful to treat or prevent conditions where suppression of the immune system or agonism of the S1P1 receptor is in order, such as diseases and disorders mediated by lymphocytes, transplant rejection, autoimmune diseases and disorders, inflammatory diseases and disorders, and conditions that have an underlying defect in vascular integrity or that relate to angiogenesis such as may be pathologic.

In one embodiment, the present invention encompasses compounds which are agonists of the S1P1 receptor having good overall physical properties and biological activities and having an effectiveness that is substantially at least that of prior compounds with activity at the S1P1 receptor.

Citation of any reference throughout this application is not to be construed as an admission that such reference is prior art to the present application.

### SUMMARY OF THE INVENTION

The present invention encompasses compounds of Formula (Ia) and pharmaceutically acceptable salts, solvates and hydrates thereof:

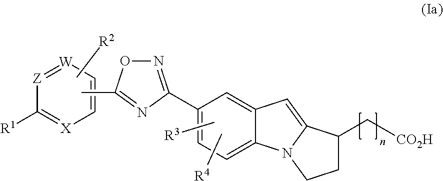

wherein:

n is 0 or 1;

W is N or CR⁵;

Z is N or CR⁶;

X is N or CR⁷; provided that W, Z and X are not all N;

R¹, R², R⁵, R⁶ and R⁷ are each independently selected from the group consisting of H, C₁-C₆ acyl, C₁-C₆ acyloxy, C₁-C₆ alkoxy, C₁-C₆ alkoxycarbonylamino, C₁-C₆ alkyl, C₂-C₆ alkynyl, C₁-C₆ alkylamino, C₂-C₈ dialkylamino, C₁-C₆ alkylcarboxamide, C₁-C₆ alkylsulfonamide, C₁-C₆ alkylsulfinyl, C₁-C₆ alkylsulfonyl, C₁-C₆ alkylthio, C₁-C₆ alkylureyl, amino, carbo-C₁-C₆-alkoxy, carboxamide, carboxy, cyano, C₃-C₇ cycloalkyl, C₃-C₇ cycloalkyloxy, C₃-C₇ cycloalkylthio, C₃-C₇ cycloalkylsulfinyl, C₃-C₇ cycloalkylsulfonyl, C₂-C₆ dialkylcarboxamide, C₁-C₆ haloalkoxy, C₁-C₆ haloalkyl, C₁-C₆ haloalkylsulfinyl, C₁-C₆ haloalkylsulfonyl, halogen, heteroaryl, heterocyclyl, hydroxyl, nitro and sulfonamide, wherein C₁-C₆ alkoxy, C₁-C₆ alkylthio, C₁-C₆ alkyl, C₂-C₆ alkynyl and heteroaryl are optionally substituted with one substituent selected from the group consisting of C₁-C₆ alkoxy, carbo-C₁-C₆-alkoxy, cyano, C₃-C₇ cycloalkyl;halogen and phenyl, or

two adjacent groups selected from R¹, R², R⁵, R⁶ and R⁷ together with the atoms to which they are both bonded form a five or six member heterocyclyl ring optionally substituted with one or two halogen atoms; and

R³ and R⁴ are each independently selected from the group consisting of H, C₁-C₂ alkyl, fluoro and chloro.

Compounds of the invention encompass compounds which are S1P1 receptor agonists having at least immunosuppressive, anti-inflammatory and/or hemostatic activities, e.g. by virtue of modulating leukocyte trafficking, sequestering lymphocytes in secondary lymphoid tissues, and/or enhancing vascular integrity.

S1P1 receptor agonists are useful to treat or prevent conditions where suppression of the immune system or agonism of S1P1 receptor is in order, such as diseases and disorders mediated by lymphocytes, transplant rejection, autoimmune diseases and disorders, inflammatory diseases and disorders (e.g., acute and chronic inflammatory conditions), cancer, and conditions that have an underlying defect in vascular integrity or that are associated with angiogenesis such as may be pathologic (e.g., as may occur in inflammation, tumor development and atherosclerosis). Such conditions where suppression of the immune system or agonism of S1P1 receptor is in order include diseases and disorders mediated by lymphocytes, conditions that have an underlying defect in vascular integrity, autoimmune diseases and disorders, inflammatory diseases and disorders (e.g., acute and chronic inflammatory conditions), (acute or chronic) rejection of cells, tissue or solid organ grafts, arthritis including psoriatic arthritis and rheumatoid arthritis, diabetes including type I diabetes, demyelinating disease including multiple sclerosis, ischemia-reperfusion injury including renal and cardiac ischemia-reperfusion injury, inflammatory skin disease including psoriasis, atopic dermatitis, acne, hyperproliferative skin disease including acne, inflammatory bowel disease including Crohn's disease and ulcerative colitis, systemic lupus erythematosis, asthma, uveitis, myocarditis, allergy, atherosclerosis, brain inflammation including Alzheimer's disease and brain inflammatory reaction following traumatic brain injury, central nervous system disease including spinal cord injury or cerebral infarction, pathologic angiogenesis including as may occur in primary and metastatic tumor growth, rheumatoid arthritis, diabetic retinopathy and atherosclerosis, cancer, chronic pulmonary disease, acute lung injury, acute respiratory disease syndrome, sepsis, ischemic injury including myocardial infarction and stroke, and the like.

One aspect of the present invention pertains to pharmaceutical compositions comprising a compound of the present invention and a pharmaceutically acceptable carrier.

One aspect of the present invention pertains to methods for treating a disorder associated with the S1P1 receptor in an individual comprising administering to the individual in need thereof a therapeutically effective amount of a compound of the present invention or a pharmaceutical composition thereof.

One aspect of the present invention pertains to methods for treating a disease or disorder mediated by lymphocytes in an individual comprising administering to the individual in need thereof a therapeutically effective amount of a compound of the present invention or a pharmaceutical composition thereof.

One aspect of the present invention pertains to methods for treating an autoimmune disease or disorder in an individual comprising administering to the individual in need thereof a therapeutically effective amount of a compound of the present invention or a pharmaceutical composition thereof.

One aspect of the present invention pertains to methods for treating an inflammatory disease or disorder in an individual comprising administering to the individual in need thereof a therapeutically effective amount of a compound of the present invention or a pharmaceutical composition thereof.

One aspect of the present invention pertains to methods for treating cancer in an individual comprising administering to the individual in need thereof a therapeutically effective amount of a compound of the present invention or a pharmaceutical composition thereof.

One aspect of the present invention pertains to methods for treating a disorder in an individual comprising administering to the individual in need thereof a therapeutically effective amount of a compound of the present invention or a pharmaceutical composition thereof, wherein said disorder is selected from the group consisting of psoriasis, rheumatoid arthritis, Crohn's disease, transplant rejection, multiple sclerosis, systemic lupus erythematosus, ulcerative colitis, type I diabetes, sepsis, myocardial infarction, ischemic stroke and acne.

One aspect of the present invention pertains to methods for treating psoriasis in an individual comprising administering to the individual in need thereof a therapeutically effective amount of a compound of the present invention or a pharmaceutical composition thereof.

One aspect of the present invention pertains to methods for treating rheumatoid arthritis in an individual comprising administering to the individual in need thereof a therapeutically effective amount of a compound of the present invention or a pharmaceutical composition thereof.

One aspect of the present invention pertains to methods for treating Crohn's disease in an individual comprising administering to the individual in need thereof a therapeutically effective amount of a compound of the present invention or a pharmaceutical composition thereof.

One aspect of the present invention pertains to methods for treating transplant rejection in an individual comprising administering to the individual in need thereof a therapeutically effective amount of a compound of the present invention or a pharmaceutical composition thereof.

One aspect of the present invention pertains to methods for treating multiple sclerosis in an individual comprising administering to the individual in need thereof a therapeutically effective amount of a compound of the present invention or a pharmaceutical composition thereof.

One aspect of the present invention pertains to methods for treating systemic lupus erythematosus in an individual comprising administering to the individual in need thereof a therapeutically effective amount of a compound of the present invention or a pharmaceutical composition thereof.

One aspect of the present invention pertains to methods for treating ulcerative colitis in an individual comprising administering to the individual in need thereof a therapeutically effective amount of a compound of the present invention or a pharmaceutical composition thereof.

One aspect of the present invention pertains to methods for treating type I diabetes in an individual comprising administering to the individual in need thereof a therapeutically effective amount of a compound of the present invention or a pharmaceutical composition thereof.

One aspect of the present invention pertains to methods for treating acne in an individual comprising administering to the individual in need thereof a therapeutically effective amount of a compound of the present invention or a pharmaceutical composition thereof.

One aspect of the present invention pertains to methods for treating a disease or disorder associated with the S1P1 receptor in an individual comprising administering to the individual in need thereof a therapeutically effective amount of a compound of the present invention or a pharmaceutical composition thereof, wherein said disorder is selected from the group consisting of a disease or disorder mediated by lymphocytes, an autoimmune disease or disorder, an inflammatory disease or disorder, cancer, psoriasis, rheumatoid arthritis, Crohn's disease, transplant rejection, multiple sclerosis, systemic lupus erythematosus, ulcerative colitis, type I diabetes, sepsis, myocardial infarction, ischemic stroke and acne.

One aspect of the present invention pertains to methods for treating a disease or disorder associated with the S1P1 receptor associated with the S1P1 receptor in an individual comprising administering to the individual in need thereof a therapeutically effective amount of a compound of the present invention or a pharmaceutical composition thereof, wherein said disorder associated with the S1P1 receptor is a microbial infection or disease or a viral infection or disease.

One aspect of the present invention pertains to methods for treating gastritis in an individual comprising administering to the individual in need thereof a therapeutically effective amount of a compound of the present invention or a pharmaceutical composition thereof.

One aspect of the present invention pertains to methods for treating polymyositis in an individual comprising administering to the individual in need thereof a therapeutically effective amount of a compound of the present invention or a pharmaceutical composition thereof.

One aspect of the present invention pertains to methods for treating thyroiditis in an individual comprising administering to the individual in need thereof a therapeutically effective amount of a compound of the present invention or a pharmaceutical composition thereof.

One aspect of the present invention pertains to methods for treating vitiligo in an individual comprising administering to the individual in need thereof a therapeutically effective amount of a compound of the present invention or a pharmaceutical composition thereof.

One aspect of the present invention pertains to methods for treating hepatitis in an individual comprising administering to the individual in need thereof a therapeutically effective amount of a compound of the present invention or a pharmaceutical composition thereof.

One aspect of the present invention pertains to methods for treating biliary cirrhosis in an individual comprising administering to the individual in need thereof a therapeutically effective amount of a compound of the present invention or a pharmaceutical composition thereof.

One aspect of the present invention pertains to the use of compounds of the present invention in the manufacture of a medicament for the treatment of an S1P1 receptor-associated disorder.

One aspect of the present invention pertains to the use of compounds of the present invention in the manufacture of a medicament for the treatment of a disease or disorder mediated by lymphocytes.

One aspect of the present invention pertains to the use of compounds of the present invention in the manufacture of a medicament for the treatment of an autoimmune disease or disorder.

One aspect of the present invention pertains to the use of compounds of the present invention in the manufacture of a medicament for the treatment of an inflammatory disease or disorder.

One aspect of the present invention pertains to the use of compounds of the present invention in the manufacture of a medicament for the treatment of cancer.

One aspect of the present invention pertains to the use of compounds of the present invention in the manufacture of a medicament for the treatment of an S1P1 receptor-associated disorder selected from the group consisting of psoriasis, rheumatoid arthritis, Crohn's disease, transplant rejection, multiple sclerosis, systemic lupus erythematosus, ulcerative colitis, type I diabetes, sepsis, myocardial infarction, ischemic stroke and acne.

One aspect of the present invention pertains to the use of compounds of the present invention in the manufacture of a medicament for the treatment of psoriasis.

One aspect of the present invention pertains to the use of compounds of the present invention in the manufacture of a medicament for the treatment of rheumatoid arthritis.

One aspect of the present invention pertains to the use of compounds of the present invention in the manufacture of a medicament for the treatment of Crohn's disease.

One aspect of the present invention pertains to the use of compounds of the present invention in the manufacture of a medicament for the treatment of transplant rejection.

One aspect of the present invention pertains to the use of compounds of the present invention in the manufacture of a medicament for the treatment of multiple sclerosis.

One aspect of the present invention pertains to the use of compounds of the present invention in the manufacture of a medicament for the treatment of systemic lupus erythematosus.

One aspect of the present invention pertains to the use of compounds of the present invention in the manufacture of a medicament for the treatment of ulcerative colitis.

One aspedt of the present invention pertains to the use of compounds of the present invention in the manufacture of a medicament for the treatment of type I diabetes.

One aspect of the present invention pertains to the use of compounds of the present invention in the manufacture of a medicament for the treatment of acne.

One aspect of the present invention pertains to the use of a compound of the present invention in the manufacture of a medicament for the treatment of an S1P1 receptor-associated disorder selected from the group consisting of a disease or disorder mediated by lymphocytes, an autoimmune disease or disorder, an inflammatory disease or disorder, cancer, psoriasis, rheumatoid arthritis, Crohn's disease, transplant rejection, multiple sclerosis, systemic lupus erythematosus, ulcerative colitis, type I diabetes, sepsis, myocardial infarction, ischemic stroke and acne.

One aspect of the present invention pertains to the use of a compound of the present invention in the manufacture of a medicament for the treatment of a S1P1 receptor associated disorder wherein said S1P1 receptor associated disorder is a microbial infection or disease or a viral infection or disease.

One aspect of the present invention pertains to the use of compounds of the present invention in the manufacture of a medicament for the treatment of gastritis.

One aspect of the present invention pertains to the use of compounds of the present invention in the manufacture of a medicament for the treatment of polymyositis.

One aspect of the present invention pertains to the use of compounds of the present invention in the manufacture of a medicament for the treatment of, thyroiditis.

One aspect of the present invention pertains to the use of compounds of the present invention in the manufacture of a medicament for the treatment of vitiligo.

One aspect of the present invention pertains to the use of compounds of the present invention in the manufacture of a medicament for the treatment of hepatitis.

One aspect of the present invention pertains to the use of compounds of the present invention in the manufacture of a medicament for the treatment of biliary cirrhosis.

One aspect of the present invention pertains to compounds of the present invention for use in a method of treatment of the human or animal body by therapy.

One aspect of the present invention pertains to compounds of the present invention for use in a method for the treatment of an S1P1 receptor-associated disorder.

One aspect of the present invention pertains to compounds of the present invention for use in a method for the treatment of a disease or disorder mediated by lymphocytes.

One aspect of the present invention pertains to compounds of the present invention for use in a method for the treatment of an autoimmune disease or disorder.

One aspect of the present invention pertains to compounds of the present invention for use in a method for the treatment of an inflammatory disease or disorder.

One aspect of the present invention pertains to compounds of the present invention for use in a method for the treatment of cancer.

One aspect of the present invention pertains to compounds of the present invention for use in a method for the treatment of an S1P1 receptor-associated disorder selected from the group consisting of psoriasis, rheumatoid arthritis, Crohn's disease, transplant rejection, multiple sclerosis, systemic lupus erythematosus, ulcerative colitis, type I diabetes, sepsis, myocardial infarction, ischemic stroke and acne.

One aspect of the present invention pertains to compounds of the present invention for use in a method for the treatment of psoriasis.

One aspect of the present invention pertains to compounds of the present invention for use in a method for the treatment of rheumatoid arthritis.

One aspect of the present invention pertains to compounds of the present invention for use in a method for the treatment of Crohn's disease.

One aspect of the present invention pertains to compounds of the present invention for use in a method for the treatment of transplant rejection.

One aspect of the present invention pertains to compounds of the present invention for use in a method for the treatment of multiple sclerosis.

One aspect of the present invention pertains to compounds of the present invention for use in a method for the treatment of systemic lupus erythematosus.

One aspect of the present invention pertains to compounds of the present invention for use in a method for the treatment of ulcerative colitis.

One aspect of the present invention pertains to compounds of the present invention for use in a method for the treatment of type I diabetes.

One aspect of the present invention pertains to compounds of the present invention for use in a method for the treatment of acne.

One aspect of the present invention pertains to compounds of the present invention for use in a method for the treatment of an S1P1 receptor-associated disorder selected from the group consisting of a disease or disorder mediated by lymphocytes, an autoimmune disease or disorder, an inflammatory disease or disorder, cancer, psoriasis, rheumatoid arthritis, Crohn's disease, transplant rejection, multiple sclerosis, systemic lupus erythematosus, ulcerative colitis, type I diabetes, sepsis, myocardial infarction, ischemic stroke and acne.

One aspect of the present invention pertains to compounds of the present invention for use in a method for the treatment of a S1P1 receptor associated disorder wherein said S1P1 receptor associated disorder is a microbial infection or disease or a viral infection or disease.

One aspect of the present invention pertains to compounds of the present invention for use in a method for the treatment of gastritis.

One aspect of the present invention pertains to compounds of the present invention for use in a method for the treatment of polymyositis.

One aspect of the present invention pertains to compounds of the present invention for use in a method for the treatment of thyroiditis.

One aspect of the present invention pertains to compounds of the present invention for use in a method for the treatment of vitiligo.

One aspect of the present invention pertains to compounds of the present invention for use in a method for the treatment of hepatitis.

One aspect of the present invention pertains to compounds of the present invention for use in a method for the treatment of biliary cirrhosis.

One aspect of the present invention pertains to processes for preparing a composition comprising admixing a compound of the present invention and a pharmaceutically acceptable carrier.

These and other aspects of the invention disclosed herein will be set forth in greater detail as the patent disclosure proceeds.

### BRIEF DESCRIPTION OF THE DRAWINGS

FIG. 1 shows the cellular/functional Ca²⁺ assay for agonist activity at the S1P3 receptor. The calcium ionophore A23187 shows pronounced agonist activity while Compound 2 (Enantiomer 1), a representative compound of the present invention, shows substantially little to no activity.

FIG. 2 shows the ability for Compound 1 to lower peripheral lymphocytes in the mouse compared to vehicle.

FIG. 3 shows the results of an experiment which measured the ability of the 2^{nd} enantiomer of Compound 2 (isolated after resolution of compound 2 by HPLC, with a retention time of 14.9 min per the conditions reported in Example 1.25) to have efficacy in experimental autoimmune encephalomyelitis (EAE) compared to vehicle.

FIG. 4 shows the results of an experiment which measured the ability of three different doses of the 2^{nd} enantiomer of Compound 2 (isolated after resolution of compound 2 by HPLC, with a retention time of 14.9 min per the conditions reported in Example 1.25) to reduce the mean ankle diameter in rats compared to vehicle.

FIG. 5 shows the results of an experiment which measured the ability of three different doses of the 2^{nd} enantiomer of Compound 2 (isolated after resolution of compound 2 by HPLC, with a retention time of 14.9 min per the conditions reported in Example 1.25) to lower allograft rejection in mice compared to vehicle.

FIG. 6 shows the results of an experiment which measured the ability of the 2^{nd} enantiomer of Compound 2 (isolated after resolution of compound 2 by HPLC, with a retention time of 14.9 min per the conditions reported in Example 1.25) to lower the blood glucose concentration in mice compared to vehicle.

FIG. 7 shows a general synthetic scheme for the preparation of N-hydroxy-carbamimidoyl intermediates useful in the preparation of Compounds of Formula (Ia) wherein “n” is 1.

FIG. 8 shows a general synthetic scheme for the preparation of N-hydroxy-carbamimidoyl intermediates useful in the preparation of Compounds of Formula (Ia) wherein “n” is 0.

FIG. 9 shows a general synthetic scheme for the preparation of N-hydroxy-carbamimidoyl intermediates useful in the preparation of Compounds of Formula (Ia) wherein “n” is 0.

FIG. 10 shows a general synthetic scheme for the preparation of compounds of Formula (Ia). The synthetic scheme shows the coupling of carboxylic acids or acid chlorides with N-hydroxy-carbamimidoyl intermediates and subsequent formation of the oxadiazole ring to provide ester intermediates. The esters can be converted to Compounds of Formula (Ia) using methods know in the art, for example, t-buyl esters are converted to carboxylic acids by treatment with an acid, such as TFA in the presence or absence of thioanisole, alkyl esters are converted to carboxylic acids by treatment with a base, such as LiOH, NaOH, KOH and the like.

FIG. 11 shows three general routes for modifying the aryl and heteroaryl ring. The first route shows adding alcohols to fluoro or chloro pyridine to provide ethers. The second route shows the formation of ethers using PPh₃ and DEAD or DIAD. The third route shows Pd(0) catalyzed alkyl couplings. Each route provides esters that are subsequently converted to Compounds of Formula (Ia).

FIG. 12 shows two general routes for modifying the aryl and heteroaryl ring present in ester intermediates. The first route shows Pd(0) catalyzed heteroaryl coupling to provide ethers. The second route shows Pd(0) catalyzed couplings to give alkynes and can optionally be reduced to provide the corresponding alkyl group. Each route provides esters that are subsequently converted to Compounds of Formula (Ia).

### DETAILED DESCRIPTION OF THE INVENTION

### Definitions

For clarity and consistency, the following definitions will be used throughout this patent document.

The term “agonist” is intended to mean a moiety that interacts with and activates a G-protein-coupled receptor, such as the S1P1 receptor, and can thereby initiate a physiological or pharmacological response characteristic of that receptor. For example, an agonist activites an intracellular response upon binding to the receptor, or enhances GTP binding to a membranes. In certain embodiments, an agonist in the invention is an S1P1 receptor agonist that is capable of facilitating sustained S1P1 receptor internalilzation (see e.g., Matloubian et al., Nature, 427, 355, 2004).

The term “antagonist” is intended to mean a moietiy that competitively binds to the receptor at the same site as an agonist (for example, the endogenous ligand), but which does not activate the intracellular response initiated by the active form of the receptor and can thereby inhibit the intracellular responses by an agonist or partial agonist. An antagonist does not diminish the baseline intracellular response which is observed in the absence of an agonist or partial agonist.

The term “in need of treatment” and the term “in need thereof” when referring to treatment are used interchangeably to mean a judgment made by a caregiver (e.g. physician, nurse, nurse practitioner, etc. in the case of humans; veterinarian in the case of animals, including non-human mammals) that an individual or animal requires or will benefit from treatment. This judgment is made based on a variety of factors that are in the realm of a caregiver's expertise, but that includes the knowledge that the individual or animal is ill, or will become ill, as the result of a disease, condition or disorder that is treatable by the compounds of the invention. Accordingly, the compounds of the invention can be used in a protective or preventive manner; or compounds of the invention can be used to alleviate, inhibit or ameliorate the disease, condition or disorder.

The term “individual” is intended to mean any animal, including mammals, preferably mice, rats, other rodents, rabbits, dogs, cats, swine, cattle, sheep, horses, or primates and most preferably humans.

The term “inverse agonist” is intended to mean a moiety that binds to the endogenous form of the receptor or to the constitutively activated form of the receptor and which inhibits the baseline intracellular response initiated by the active form of the receptor below the normal base level of activity which is observed in the absence of an agonist or partial agonist, or decrease GTP binding to a membrane. In some embodiments, the baseline intracellular response is inhibited in the presence of the inverse agonist by at least 30%, by at least 50%, or by at least 75%, as compared with the baseline response in the absence of the inverse agonist.

The term “modulate or modulating” is intended to mean an increase or decrease in the amount, quality, response or effect of a particular activity, function or molecule.

The term “pharmaceutical composition” is intended to mean a composition comprising at least one active ingredient, including but not limited to, salts, solvates and hydrates of compounds of the present invention, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

The term “therapeutically effective amount” is intended to mean the amount of active compound or pharmaceutical agent that elicits the biological or medicinal response in a tissue, system, animal, individual or human that is being sought by a researcher, veterinarian, medical doctor or other clinician or caregiver, or by an individual, which includes one or more of the following:

(1) Preventing the disease, for example, preventing a disease, condition or disorder in an individual that may be predisposed to the disease, condition or disorder but does not yet experience or display the pathology or symptomatology of the disease,

(2) Inhibiting the disease, for example, inhibiting a disease, condition or disorder in an individual that is experiencing or displaying the pathology or symptomatology of the disease, condition or disorder (i.e., arresting further development of the pathology and/or symptomatology); and

(3) Ameliorating the disease, for example, ameliorating a disease, condition or disorder in an individual that is experiencing or displaying the pathology or symptomatology of the disease, condition or disorder (i.e., reversing the pathology and/or symptomatology).

### Chemical Group, Moiety or Radical

The term “C₁-C₆ acyl” is intended to mean a C₁-C₆ alkyl radical attached to the carbon of a carbonyl group wherein the definition of alkyl has the same definition as described herein; some examples include, but are not limited to, acetyl, propionyl, n-butanoyl, sec-butanoyl, pivaloyl, pentanoyl and the like.

The term “C₃-C₆ acyloxy” is intended to mean an acyl radical attached to an oxygen atom wherein acyl has the same definition has described herein; some embodiments are when acyloxy is C₁-C₅ acyloxy, some embodiments are when acyloxy is C₁-C₆ acyloxy. Some examples include, but are not limited to, acetyloxy, propionyloxy, butanoyloxy, iso-butanoyloxy, pentanoyloxy, hexanoyloxy and the like.

The term “C₁-C₆ alkoxy” is intended to mean a C₁-C₆ alkyl radical, as defined herein, attached directly to an oxygen atom, some embodiments are 1 to 5 carbons, some embodiments are 1 to 4 carbons, some embodiments are 1 to 3 carbons and some embodiments are 1 or 2 carbons. Examples include methoxy, ethoxy, n-propoxy, iso-propoxy, n-butoxy, t-butoxy, iso-butoxy, sec-butoxy and the like.

The term “C₁-C₆ alkoxycarbonylamino” is intended to mean a single C₁-C₆ alkoxy group attached to the carbon of an amide group wherein alkoxy has the same definition as found herein. The C₁-C₆ alkoxycarbonylamino group may be represented by the following:

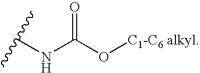

The term “C₁-C₆ alkyl” is intended to mean a straight or branched carbon radical containing 1 to 6 carbons. Some embodiments are 1 to 5 carbons, some embodiments are 1 to 4 carbons, some embodiments are 1 to 3 carbons and some embodiments are 1 or 2 carbons. Examples of an alkyl include, but not limited to, methyl, ethyl, n-propyl, iso-propyl, n-butyl, sec-butyl, iso-butyl, t-butyl, pentyl, iso-pentyl, t-pentyl, neo-pentyl, 1-methylbutyl [i.e., —CH(CH₃)CH₂CH₂CH₃], 2-methylbutyl [i.e., —CH₂CH(CH₃)CH₂CH₃], n-hexyl and the like.

The term “C₁-C₆ alkylcarboxamido” or “C₁-C₆ alkylcarboxamide” is intended to mean a single C₁-C₆ alkyl group attached to either the carbon or the nitrogen of an amide group, wherein alkyl has the same definition as found herein. The C₁-C₆ alkylcarboxamido group may be represented by the following:

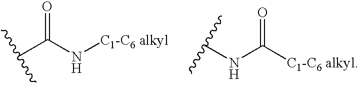

Examples include, but are not limited to, N-methylcarboxamide, N-ethylcarboxamide, N-n-propylcarboxamide, N-iso-propylcarboxamide, N-n-butylcarboxamide, N-sec-butylcarboxamide, N-iso-butylcarboxamide, N-t-butylcarboxamide and the like.

The term “C₁-C₆ alkylsulfinyl” is intended to mean a C₁-C₆ alkyl radical attached to the sulfur of a sulfoxide radical having the formula: —S(O)— wherein the alkyl radical has the same definition as described herein. Examples include, but are not limited to, methylsulfinyl, ethylsulfinyl, n-propylsulfinyl, iso-propylsulfinyl, n-butylsulfinyl, sec-butylsulfinyl, iso-butylsulfinyl, t-butylsulfinyl and the like.

The term “C₁-C₆ alkylsulfonamide” is intended to mean the groups shown below:

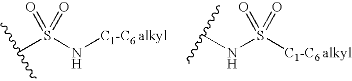

wherein C₁-C₆ alkyl has the same definition as described herein.

The term “C₁-C₆ alkylsulfonyl” is intended to mean a C₁-C₆ alkyl radical attached to the sulfur of a sulfone radical having the formula: —S(O)₂— wherein the alkyl radical has the same definition as described herein. Examples include, but are not limited to, methylsulfonyl, ethylsulfonyl, n-propylsulfonyl, iso-propylsulfonyl, n-butylsulfonyl, sec-butylsulfonyl, iso-butylsulfonyl, t-butylsulfonyl and the like.

The term “C₁-C₆ alkylthio” is intended to mean a C₁-C₆ alkyl radical attached to a sulfur atom (i.e., —S—) wherein the alkyl radical has the same definition as described herein. Examples include, but are not limited to, methylsulfanyl (i.e., CH₃S—), ethylsulfanyl, n-propylsulfanyl, iso-propylsulfanyl, n-butylsulfanyl, sec-butylsulfanyl, iso-butylsulfanyl, t-butylsulfanyl, and the like.

The term “C₁-C₆ alkylureyl” is intended to mean the group of the formula: —NC(O)N— wherein one or both of the nitrogens are substituted with the same or different C₁-C₆alkyl group wherein alkyl has the same definition as described herein. Examples of an alkylureyl include, but are not limited to, CH₃NHC(O)NH—, NH₂C(O)NCH₃—, (CH₃)₂NC(O)NH—, (CH₃)₂NC(O)NCH₃—, CH₃CH₂NHC(O)NH—, CH₃CH₂NHC(O)NCH₃— and the like.

The term “amino” is intended to mean the group —NH₂.

The term “C₁-C₆ alkylamino” is intended to mean one alkyl radical attached to a —NH— radical wherein the alkyl radical has the same meaning as described herein. Some examples include, but are not limited to, methylamino, ethylamino, n-propylamino, iso-propylamino, n-butylamino, sec-butylamino, iso-butylamino, t-butylamino and the like. Some embodiments are “C₁-C₂ alkylamino.”

The term “aryl” is intended to mean an aromatic ring radical containing 6 to 10 ring carbons. Examples include phenyl and naphthyl.

The term “carbo-C₁-C₆-alkoxy” is intended to mean a C₁-C₆ alkyl ester of a carboxylic acid, wherein the alkyl group is as defined herein. Examples include, but are not limited to, carbomethoxy [—C(═O)OCH₃], carboethoxy, carbopropoxy, carboisopropoxy, carbobutoxy, carbo-sec-butoxy, carbo-iso-butoxy, carbo-t-butoxy, carbo-n-pentoxy, carbo-iso-pentoxy, carbo-t-pentoxy, carbo-neo-pentoxy, carbo-n-hexyloxy and the like.

The term “C₂-C₆ alkynyl” is intended to mean a radical containing at least one carbon-carbon triple bond and 2 to 6 carbons. Some examples include, but are not limited to, ethynyl, 1-propynyl, 2-propynyl, 1-butynyl, 2-butynyl, and the like.

The term “carboxamide” is intended to mean the group —CONH₂.

The term “carboxy” or “carboxyl” is intended to mean the group —CO₂H; also referred to as a carboxylic acid group.

The term “cyano” is intended to mean the group —CN.

The term “C₃-C₇ cycloalkyl” is intended to mean a saturated ring radical containing 3 to 7 carbons; some embodiments contain 3 to 6 carbons; some embodiments contain 3 to 5 carbons; some embodiments contain 5 to 7 carbons; some embodiments contain 3 to 4 carbons. Examples include cyclopropyl, cyclobutyl, cyclopentyl, cyclohexyl, cycloheptyl and the like.

The term “C₃-C₇ cycloalkyloxy” is intended to mean a saturated ring radical containing 3 to 7 carbons directly bonded to an oxygen atom. Some examples include cyclopropyl-O—, cyclobutyl-O—, cyclopentyl-O—, cyclohexyl-O—, and the like.

The term “C₃-C₇ cycloalkylthio” is intended to mean a saturated ring radical containing 3 to 7 carbons directly bonded to a sulfur atom. Some examples include cyclopropyl-S—, cyclobutyl-S—, cyclopentyl-S—, cyclohexyl-S—, and the like.

The term “C₃-C₇ cycloalkylsulfinyl” is intended to mean a saturated ring radical containing 3 to 7 carbons directly bonded to a sulfoxide group. Some examples include cyclopropyl-S(O)—, cyclobutyl-S(O)—, cyclopentyl-S(O)—, cyclohexyl-S(O)—, and the like.

The term “C₃-C₇ cycloalkylsulfonyl” is intended to mean a saturated ring radical containing 3 to 7 carbons directly bonded to a sulfoxide group. Some examples include cyclopropyl-S(O)₂—, cyclobutyl-S(O)₂—, cyclopentyl-S(O)₂—, cyclohexyl-S(O)₂—, and the like.

The term “C₂-C₈ dialkylamino” is intended to mean an amino group substituted with two of the same or different C₁-C₄ alkyl radicals wherein alkyl radical has the same definition as described herein. Some examples include, but are not limited to, dimethylamino, methylethylamino, diethylamino, methylpropylamino, methylisopropylamino, ethylpropylamino, ethylisopropylamino, dipropylamino, propylisopropylamino and the like. Some embodiments are C₂-C₄ dialkylamino.

The term “C₂-C₆ dialkylcarboxamido” or “C₂-C₆ dialkylcarboxamide” is intended to mean two alkyl radicals, that are the same or different, attached to an amide group, wherein alkyl has the same definition as described herein. A C₂-C₆ dialkylcarboxamido may be represented by the following groups:

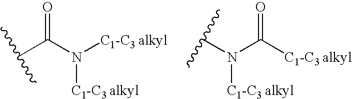

wherein C₁-C₃ has the same definition as described herein. Examples of a dialkylcarboxamide include, but are not limited to, N,N-dimethylcarboxamide, N-methyl-N-ethylcarboxamide, N,N-diethylcarboxamide, N-methyl-N-isopropylcarboxamide and the like.

The term “C₁-C₆ haloalkoxy” is intended to mean a C₁-C₆ haloalkyl, as defined herein, which is directly attached to an oxygen atom. Examples include, but are not limited to, difluoromethoxy, trifluoromethoxy, 2,2,2-trifluoroethoxy, pentafluoroethoxy and the like.

The term “C₁-C₆ haloalkyl” is intended to mean an C₁-C₆ alkyl group, as defined herein, wherein the alkyl is substituted with one halogen up to fully substituted and a fully substituted C₁-C₆ haloalkyl can be represented by the formula CₐL₂ₐ₊₁ wherein L is a halogen and “a” is 1, 2, 3, 4, 5 or 6. When more than one halogen is present then they may be the same or different and selected from the group consisting of F, Cl, Br and I, some embodiments are 1 to 5 carbons, some embodiments are 1 to 4 carbons, some embodiments are 1 to 3 carbons and some embodiments are 1 or 2 carbons. Examples of haloalkyl groups include, but are not limited to, fluoromethyl, difluoromethyl, trifluoromethyl, chlorodifluoromethyl, 2,2,2-trifluoroethyl, pentafluoroethyl and the like.

The term “C₁-C₆ haloalkylsulfinyl” is intended to mean a C₁-C₆ haloalkyl radical attached to the sulfur atom of a sulfoxide group having the formula: —S(O)— wherein the haloalkyl radical has the same definition as described herein. Examples include, but are not limited to, trifluoromethylsulfinyl, 2,2,2-trifluoroethylsulfinyl, 2,2-difluoroethylsulfinyl and the like.

The term “C₁-C₆ haloalkylsulfonyl” is intended to mean a C₁-C₆ haloalkyl radical attached to the sulfur atom of a sulfone group having the formula: —S(O)₂— wherein haloalkyl has the same definition as described herein. Examples include, but are not limited to, trifluoromethylsulfonyl, 2,2,2-trifluoroethylsulfonyl, 2,2-difluoroethylsulfonyl and the like.

The term “halogen” or “halo” is intended to mean to a fluoro, chloro, bromo or iodo group.

The term “heteroaryl” is intended to mean an aromatic ring system containing 5 to 14 aromatic ring atoms that may be a single ring, two fused rings or three fused rings wherein at least one aromatic ring atom is a heteroatom selected from, for example, but not limited to, the group consisting of O, S and N wherein the N can be optionally substituted with H, C₁-C₄ acyl or C₁-C₄ alkyl. Some embodiments contain 5 or 6 ring atoms for example furanyl, thienyl, pyrrolyl, imidazolyl, oxazolyl, thiazolyl, isoxazolyl, pyrazolyl, isothiazolyl, oxadiazolyl, triazolyl, thiadiazolyl, pyridinyl, pyrazinyl, pyrimidinyl, pyridazinyl and triazinyl and the like. Some embodiments contain 8 to 14 ring atoms for example quinolizinyl, quinolinyl, isoquinolinyl, cinnolinyl, phthalazinyl, quinazolinyl, quinoxalinyl, triazinyl, indolyl, isoindolyl, indazolyl, indolizinyl, purinyl, naphthyridinyl, pteridinyl, carbazolyl, acridinyl. phenazinyl, phenothiazinyl, phenoxazinyl, benzoxazolyl, benzothiazolyl, 1H-benzimidazolyl, imidazopyridinyl, benzothienyl, benzofuranyl, and isobenzofuran and the like

The term “heterocyclic” or “heterocyclyl” is intended to mean a non-aromatic carbon ring containing 3 to 8 ring atoms wherein one, two or three ring atoms are heteroatoms selected from, for example, the group consisting of O, S and NH, wherein the N is optionally substituted with C₁-C₄ alkyl or as described herein and S is optionally substituted with 1 or 2 oxygens. In some embodiments, the nitrogen is optionally substituted with C₁-C₄ acyl or C₁-C₄ alkyl and ring carbon atoms are optionally substituted with oxo or a thiooxo thus forming a carbonyl or thiocarbonyl group. The heterocyclic group can be attached/bonded to any available ring atom, for example, ring carbon, ring nitrogen and the like. In some embodiments the heterocyclic group is a 3-, 4-, 5-, 6- or 7-membered ring. Examples of a heterocyclic group include, but are not limited to, aziridin-1-yl, aziridin-2-yl, azetidin-1-yl, azetidin-2-yl, azetidin-3-yl, piperidin-1-yl, piperidin-2-yl, piperidin-3-yl, piperidin-4-yl, morpholin-2-yl, morpholin-3-yl, morpholin-4-yl, piperzin-1-yl, piperzin-2-yl, piperzin-3-yl, piperzin-4-yl, pyrrolidin-1-yl, pyrrolidin-2-yl, pyrrolidin-3-yl, [1,3]-dioxolan-2-yl, thiomorpholin-4-yl, 1,4-oxazepan-4-yl, 1,1-dioxothiomorpholin-4-yl, azepan-1-yl, azepan-2-yl, azepan-3-yl, azepan-4-yl, tetrahydrofuran-2-yl, tetrahydrofuran-3-yl and the like.

The term “hydroxyl” is intended to mean the group —OH.

The term “nitro” is intended to mean the group —NO₂.

The term “phenyl” is intended to mean the group —C₆H₅.

The term “sulfonamide” is intended to mean the group —SO₂NH₂.

### Compounds of the Invention:

One aspect of the present invention pertains to certain compounds of Formula (Ia) and pharmaceutically acceptable salts, solvates and hydrates thereof:

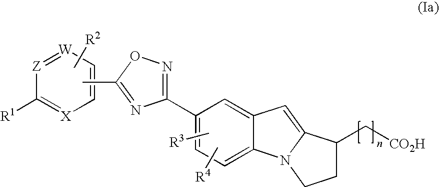

wherein:

n, R¹, R², R³, R⁴, W, Z and X have the same definitions as described herein, supra and infra.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the chemical groups represented by the variables (e.g., n, R¹, R², R³, R⁴, R⁵, R⁶, R⁷, R^{a}W, Z and X) contained within the generic chemical formulae described herein, for example, (Ia, Ic, Ie, Ig, Ii, Ik, Im, Io, Iq, IIa, IIc, IIe, IIg, IIi, IIk, IIm, IIo, IIq, IIIa) are specifically embraced by the present invention just as if each and every combination was individually explicitly recited, to the extent that such combinations embrace compounds that result in stable compounds (i.e., compounds that can be isolated, characterized and tested for biological activity). In addition, all subcombinations of the chemical groups listed in the embodiments describing such variables, as well as all subcombinations of uses and medical indications described herein, are also specifically embraced by the present invention just as if each and every subcombination of chemical groups and subcombination of uses and medical indications was individually and explicitly recited herein.

As used herein, “substituted” indicates that at least one hydrogen atom of the chemical group is replaced by a non-hydrogen substituent or group, the non-hydrogen substituent or group can be monovalent or divalent. When the substituent or group is divalent, then it is understood that this group is further substituted with another substituent or group. When a chemical group herein is “substituted” it may have up to the full valance of substitution; for example, a methyl group can be substituted with 1, 2, or 3 substituents, a methylene group can be substituted by 1 or 2 substituents, a phenyl group can be substituted with 1, 2, 3, 4, or 5 substituents, a naphthyl group can be substituted with 1, 2, 3, 4, 5, 6, or 7 substituents and the like. Likewise, “substituted with one or more substituents” refers to the substitution of a group with one substituent up to the total number of substituents physically allowed by the group. Further, when a group is substituted with more than one group they can be identical or they can be different.

Compounds of the invention also include tautomeric forms, such as keto-enol tautomers and the like. Tautomeric forms can be in equilibrium or sterically locked into one form by appropriate substitution. It is understood that the various tautomeric forms are within the scope of the compounds of the present invention.

Compounds of the invention also include all isotopes of atoms occurring in the intermediates and/or final compounds. Isotopes include those atoms having the same atomic number but different mass numbers. For example, isotopes of hydrogen include deuterium and tritium.

It is understood and appreciated that compounds of Formula (Ia) and formulae related thereto may have one or more chiral centers and therefore can exist as enantiomers and/or diastereomers. The invention is understood to extend to and embrace all such enantiomers, diastereomers and mixtures thereof, including but not limited to racemates. It is understood that compounds of Formula (Ia) and formulae used throughout this disclosure are intended to represent all individual enantiomers and mixtures thereof, unless stated or shown otherwise.

The “n” Variable

In some embodiments, n is 1.

Some embodiments of the present invention pertain to compounds of Formula (Ic) and pharmaceutically acceptable salts, solvates and hydrates thereof:

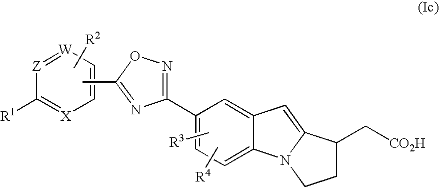

wherein each variable in Formula (Ic) has the same meaning as described herein, supra and infra.

In some embodiments, n is 0.

Some embodiments of the present invention pertain to compounds of Formula (Ie) and pharmaceutically acceptable salts, solvates and hydrates thereof:

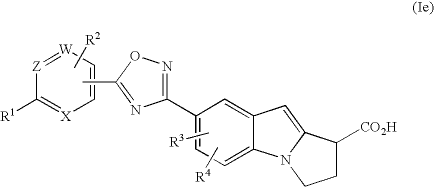

wherein each variable in Formula (Ie) has the same meaning as described herein, supra and infra.

### The Group R¹

In some embodiments, R¹ is selected from the group consisting of H, C₁-C₆ alkoxy, C₁-C₆ alkyl, C₁-C₆ alkylcarboxamide, cyano, C₁-C₆ haloalkoxy, C₁-C₆ haloalkyl, halogen, heteroaryl, heterocyclyl and hydroxyl.

In some embodiments, R¹ is selected from the group consisting of H, methoxy, isopropoxy, methyl, acetamido, cyano, trifluoromethoxy, 2,2,2-trifluoroethoxy, 1,1,1-trifluoropropan-2-yloxy, trifluoromethyl, fluoro, chloro, pyrazol-1-yl, morpholino and hydroxyl.

### The Group R²

In some embodiments, R² is selected from the group consisting of H, C₁-C₆ alkoxy, C₁-C₆ alkyl and halogen.

In some embodiments, R² is selected from the group consisting of H, methoxy, methyl and chloro.

In some embodiments, R² is H.

### The Groups R³ and R⁴

In some embodiments, R³ and R⁴ are each independently selected from the group consisting of H, CH₃ and F.

It is understood that R³ and R⁴ can be bonded to any of the three available positions on the phenyl ring of the 2,3-dihydro-1H-pyrrolo[1,2-a]indole ring system, specifically these positions are C(5), C(6) and C(8) as illustrated in the formula below:

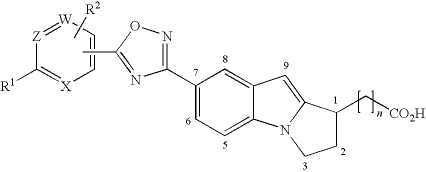

In some embodiments, R³ is H.

In some embodiments, R⁴ is H.

In some embodiments, R³ and R⁴ are both H.

Some embodiments of the present invention pertain to compounds of Formula (Ig) and pharmaceutically acceptable salts, solvates and hydrates thereof:

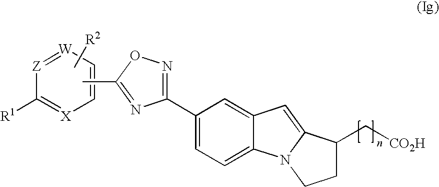

wherein each variable in Formula (Ig) has the same meaning as described herein, supra and infra.

### The Group R⁵

In some embodiments, R⁵ is selected from the group consisting of H, C₁-C₆ alkyl, C₁-C₆ haloalkyl and hydroxyl.

In some embodiments, R⁵ is selected from the group consisting of H, methyl, trifluoromethyl and hydroxyl.

In some embodiments, R⁵ is H.

### The Group R⁶

In some embodiments, R⁶ is selected from the group consisting of H, C₁-C₆ alkoxy, C₁-C₆ alkyl, cyano, C₁-C₆ haloalkoxy, C₁-C₆ haloalkyl, halogen and heteroaryl.

In some embodiments, R⁶ is selected from the group consisting of H, methoxy, methyl, cyano, trifluoromethoxy, difluoromethoxy, trifluoromethyl, bromo, tetrazol-5-yl, thiophen-2-yl and pyridin-3-yl.

In some embodiments, R⁶ is H.

### The Group R⁷

In some embodiments, R⁷ is selected from the group consisting of H, C₁-C₆ alkoxy, C₁-C₆ alkyl, cyano, C₁-C₆ haloalkoxy, C₁-C₆ haloalkyl, halogen and heteroaryl.

In some embodiments, R⁷ is selected from the group consisting of H, methoxy, methyl, cyano, trifluoromethoxy, trifluoromethyl, bromo, tetrazol-5-yl and thiophen-2-yl.

In some embodiments, R⁷ is H.

### The Groups R¹ and R⁶—Fused Ring Substitutions

In some embodiments, R¹ and R⁶ together with the atoms to which they are both bonded form a five member heterocyclyl ring optionally substituted with two halogen atoms.

In some embodiments, R¹ and R⁶ together with the atoms to which they are both bonded form a five member heterocyclyl ring containing two oxygen atoms and optionally substituted with two halogen atoms.

### The Groups R⁵ and R⁶—Fused Ring Substitutions

In some embodiments, R⁵ and R⁶ together with the atoms to which they are both bonded form a five member heterocyclyl ring substituted with two fluorine atoms.

In some embodiments, R⁵ and R⁶ together with the atoms to which they are both bonded form a five member heterocyclyl ring containing two oxygen atoms substituted with two halogen atoms.

### The Groups R¹, R², R⁵, R⁶ and R⁷

In some embodiments, R¹, R², R⁵, R⁶ and R⁷ are each independently selected from the group consisting of H, C₁-C₆ alkoxy, C₁-C₆ alkyl, C₁-C₆ alkylcarboxamide, cyano, C₁-C₆ haloalkoxy, C₁-C₆ haloalkyl, halogen, heteroaryl, heterocyclyl and hydroxyl, or

two adjacent groups selected from R¹, R², R⁵, R⁶ and R⁷ together with the atoms to which they are both bonded form a five member heterocyclyl ring optionally substituted with two halogen atoms.

In some embodiments, R¹, R², R⁵, R⁶ and R⁷ are each independently selected from the group consisting of H, C₁-C₆ alkoxy, C₁-C₆ alkyl, C₁-C₆ alkylcarboxamide, cyano, C₁-C₆ haloalkoxy, C₁-C₆ haloalkyl, halogen, heteroaryl, heterocyclyl and hydroxyl.

In some embodiments, R¹, R², R⁵, R⁶ and R⁷ are each independently selected from the group consisting of H, methoxy, isopropoxy, methyl, acetamido, cyano, trifluoromethoxy, difluoromethoxy, 2,2,2-trifluoroethoxy, 1,1,1-trifluoropropan-2-yloxy, trifluoromethyl, fluoro, chloro, bromo, tetrazol-5-yl, pyrazol-1-yl, thiophen-2-yl, pyridin-3-yl, morpholino and hydroxyl, or

two adjacent groups selected from R¹, R², R⁵, R⁶ and R⁷ together with the atoms to which they are both bonded form a five member heterocyclyl ring containing two oxygen atoms and optionally substituted with two halogen atoms.

In some embodiments, R¹, R², R⁵, R⁶ and R⁷ are each independently selected from the group consisting of H, methoxy, isopropoxy, methyl, acetamido, cyano, trifluoromethoxy, difluoromethoxy, 2,2,2-trifluoroethoxy, 1,1,1-trifluoropropan-2-yloxy, trifluoromethyl, fluoro, chloro, bromo, tetrazol-5-yl, pyrazol-1-yl, thiophen-2-yl, pyridin-3-yl, morpholino and hydroxyl.

### Certain Combinations for Groups R¹, R², R⁵, R⁶ and R⁷

In some embodiments, R¹ is selected from the group consisting of H, C₁-C₆ alkoxy, C₁-C₆ alkyl, C₁-C₆ alkylcarboxamide, cyano, C₁-C₆ haloalkoxy, C₁-C₆ haloalkyl, halogen, heteroaryl, heterocyclyl and hydroxyl;

R² is selected from the group consisting of H, C₁-C₆ alkoxy, C₁-C₆ alkyl and halogen;

R⁵ is selected from the group consisting of H, C₁-C₆ alkyl, C₁-C₆ haloalkyl and hydroxyl;

R⁶ is selected from the group consisting of H, C₁-C₆ alkoxy, C₁-C₆ alkyl, cyano, C₁-C₆ haloalkoxy, C₁-C₆ haloalkyl, halogen and heteroaryl; and

R⁷ is selected from the group consisting of H, C₁-C₆ alkoxy, C₁-C₆ alkyl, cyano, C₁-C₆ haloalkoxy, C₁-C₆ haloalkyl, halogen and heteroaryl.

In some embodiments, R¹ is selected from the group consisting of H, methoxy, isopropoxy, methyl, acetamido, cyano, trifluoromethoxy, 2,2,2-trifluoroethoxy, 1,1,1-trifluoropropan-2-yloxy, trifluoromethyl, fluoro, chloro, pyrazol-1-yl, morpholino and hydroxyl;

R² is selected from the group consisting of H, methoxy, methyl and chloro;

R⁵ is selected from the group consisting of H, methyl, trifluoromethyl and hydroxyl;

R⁶ is selected from the group consisting of H, methoxy, methyl, cyano, trifluoromethoxy, difluoromethoxy, trifluoromethyl, bromo, tetrazol-5-yl, thiophen-2-yl, and pyridin-3-yl; and

R⁷ is selected from the group consisting of H, methoxy, methyl, cyano, trifluoromethoxy, trifluoromethyl, bromo, tetrazol-5-yl and thiophen-2-yl.

In some embodiments, R¹ is selected from the group consisting of H, methoxy, isopropoxy, methyl, acetamido, cyano, trifluoromethoxy, 2,2,2-trifluoroethoxy, 1,1,1-trifluoropropan-2-yloxy, trifluoromethyl, fluoro, chloro, pyrazol-1-yl, morpholino and hydroxyl;

R² is H;

R⁵ is H;

R⁶ is selected from the group consisting of H, methoxy, methyl, cyano, trifluoromethoxy, difluoromethoxy, trifluoromethyl, bromo, tetrazol-5-yl, thiophen-2-yl and pyridin-3-yl; and

R⁷ is selected from the group consisting of H, methoxy, methyl, cyano, trifluoromethoxy, trifluoromethyl, bromo, tetrazol-5-yl and thiophen-2-yl.

### The Variables W, Z and X

In some embodiments, W is CR⁵; Z is CR⁶; and X is CR⁷.

Some embodiments of the present invention pertain to compounds of Formula (Ii) and pharmaceutically acceptable salts, solvates and hydrates thereof:

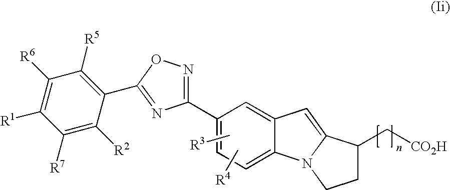

wherein each variable in Formula (Ii) has the same meaning as described herein, supra and infra.

In some embodiments, W is CR⁵; Z is CR⁶; and X is N.

Some embodiments of the present invention pertain to compounds of Formula (Ik) and pharmaceutically acceptable salts, solvates and hydrates thereof:

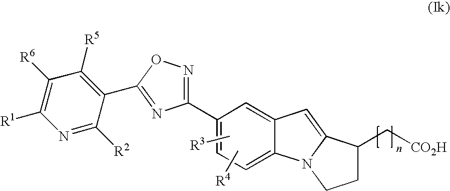

wherein each variable in Formula (Ik) has the same meaning as described herein, supra and infra.

Some embodiments of the present invention pertain to compounds of Formula (Im) and pharmaceutically acceptable salts, solvates and hydrates thereof:

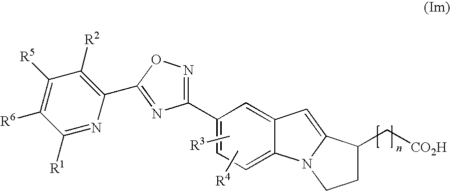

wherein each variable in Formula (Im) has the same meaning as described herein, supra and infra.

In some embodiments, W is N; Z is CR⁶; and X is N.

Some embodiments of the present invention pertain to compounds of Formula (Io) and pharmaceutically acceptable salts, solvates and hydrates thereof:

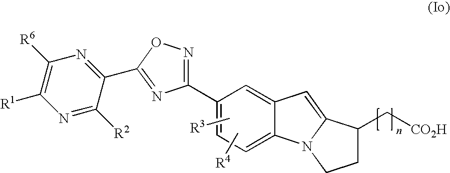

wherein each variable in Formula (Io) has the same meaning as described herein, supra and infra.

In some embodiments, W is CR⁵; Z is N; and X is CR⁷.

Some embodiments of the present invention pertain to compounds of Formula (Iq) and pharmaceutically acceptable salts, solvates and hydrates thereof:

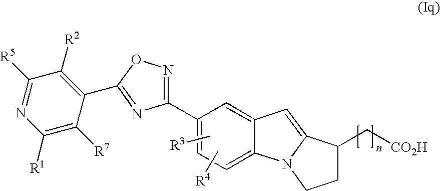

wherein each variable in Formula (Iq) has the same meaning as described herein, supra and infra.

### Certain Combinations

Some embodiments of the present invention pertain to compounds of Formula (IIa) and pharmaceutically acceptable salts, solvates and hydrates thereof:

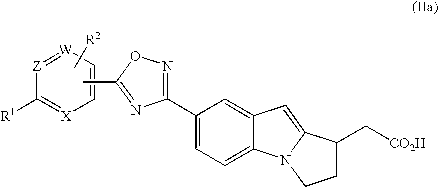

wherein:

W is N or CR⁵;

Z is N or CR⁶;

X is N or CR⁷; provided that W, Z and X are not all N, and when Z is N, then W is CR⁵ and X is CR⁷;

R¹ is selected from the group consisting of H, C₁-C₆ alkoxy, C₁-C₆ alkyl, C₁-C₆ alkylcarboxamide, cyano, C₁-C₆ haloalkoxy, C₁-C₆ haloalkyl, halogen, heteroaryl, heterocyclyl and hydroxyl;

R² is selected from the group consisting of H, C₁-C₆ alkoxy, C₁-C₆ alkyl and halogen;

R⁵ is selected from the group consisting of H, C₁-C₆ alkyl, C₁-C₆ haloalkyl and hydroxyl;

R⁶ is selected from the group consisting of H, C₁-C₆ alkoxy, C₁-C₆ alkyl, cyano, C₁-C₆ haloalkoxy, C₁-C₆ haloalkyl, halogen and heteroaryl; and

R⁷ is selected from the group consisting of H, C₁-C₆ alkoxy, C₁-C₆ alkyl, cyano, C₁-C₆ haloalkoxy, C₁-C₆ haloalkyl, halogen and heteroaryl;

or R¹ and R⁶ together with the atoms to which they are both bonded form a five member heterocyclyl ring optionally substituted with two halogen atoms;

or R⁵ and R⁶ together with the atoms to which they are both bonded form a five member heterocyclyl ring substituted with two halogen atoms.

Some embodiments of the present invention pertain to compounds of Formula (IIa) and pharmaceutically acceptable salts, solvates and hydrates thereof:

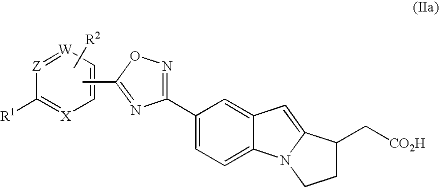

wherein:

W is N or CR⁵;

Z is N or CR⁶;

X is N or CR⁷; provided that W, Z and X are not all N, and when Z is N, then W is CR⁵ and X is CR⁷;

R¹ is selected from the group consisting of H, C₁-C₆ alkoxy, C₁-C₆ alkyl, C₁-C₆ alkylcarboxamide, cyano, C₁-C₆ haloalkoxy, C₁-C₆ haloalkyl, halogen, heteroaryl, heterocyclyl and hydroxyl;

R² is selected from the group consisting of H, C₁-C₆ alkoxy, C₁-C₆ alkyl and halogen;

R⁵ is selected from the group consisting of H, C₁-C₆ alkyl, C₁-C₆ haloalkyl and hydroxyl;

R⁶ is selected from the group consisting of H, C₁-C₆ alkoxy, C₁-C₆ alkyl, cyano, C₁-C₆ haloalkoxy, C₁-C₆ haloalkyl, halogen and heteroaryl; and

R⁷ is selected from the group consisting of H, C₁-C₆ alkoxy, C₁-C₆ alkyl, cyano, C₁-C₆ haloalkoxy, C₁-C₆ haloalkyl, halogen and heteroaryl.

Some embodiments of the present invention pertain to compounds of Formula (IIa) and pharmaceutically acceptable salts, solvates and hydrates thereof:

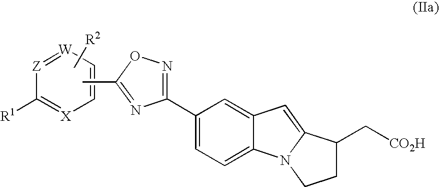

wherein:

W is N or CR⁵;

Z is N or CR⁶;

X is N or CR⁷; provided that W, Z and X are not all N, and when Z is N, then W is CR⁵ and X is CR⁷;

R¹ is selected from the group consisting of H, C₁-C₆ alkoxy, C₁-C₆ alkyl, C₁-C₆ alkylcarboxamide, cyano, C₁-C₆ haloalkoxy, C₁-C₆ haloalkyl, halogen, heteroaryl, heterocyclyl and hydroxyl;

R² is selected from the group consisting of H, methoxy, methyl and chloro;

R⁵ is selected from the group consisting of H, methyl, trifluoromethyl and hydroxyl;

R⁶ is selected from the group consisting of H, methoxy, methyl, cyano, trifluoromethoxy, difluoromethoxy, trifluoromethyl, bromo, tetrazol-5-yl, thiophen-2-yl, and pyridin-3-yl; and

R⁷ is selected from the group consisting of H, methoxy, methyl, cyano, trifluoromethoxy, trifluoromethyl, bromo, tetrazol-5-yl and thiophen-2-yl.

Some embodiments of the present invention pertain to compounds of Formula (IIa) and pharmaceutically acceptable salts, solvates and hydrates thereof:

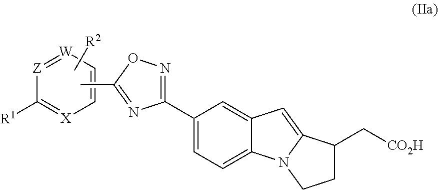

wherein:

W is N or CR⁵;

Z is N or CR⁶;

X is N or CR⁷; provided that W, Z and X are not all N, and when Z is N, then W is CR⁵ and X is CR⁷;

R¹ is selected from the group consisting of H, methoxy, isopropoxy, methyl, acetamido, cyano, trifluoromethoxy, 2,2,2-trifluoroethoxy, 1,1,1-trifluoropropan-2-yloxy, trifluoromethyl, fluoro, chloro, pyrazol-1-yl, morpholino and hydroxyl;

R² is H;

R⁵ is H;

R⁶ is selected from the group consisting of H, methoxy, methyl, cyano, trifluoromethoxy, difluoromethoxy, trifluoromethyl, bromo, tetrazol-5-yl, thiophen-2-yl and pyridin-3-yl; and

R⁷ is selected from the group consisting of H, methoxy, methyl, cyano, trifluoromethoxy, trifluoromethyl, bromo, tetrazol-5-yl and thiophen-2-yl.

Some embodiments of the present invention pertain to compounds of Formula (IIc) and pharmaceutically acceptable salts, solvates and hydrates thereof:

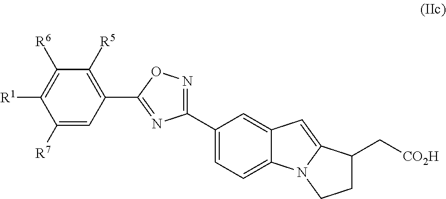

wherein:

R¹ is selected from the group consisting of H, C₁-C₆ alkoxy, C₁-C₆ haloalkoxy, halogen and hydroxyl;

R⁵ is H;

R⁶ is selected from the group consisting of cyano, C₁-C₆ haloalkoxy, C₁-C₆ haloalkyl, halogen and heteroaryl;

and

R⁷ is selected from the group consisting of H, C₁-C₆ alkoxy, cyano, C₁-C₆ haloalkoxy, C₁-C₆ haloalkyl and heteroaryl; or

two adjacent groups selected from R¹, R⁵ and R⁶ together with the atoms to which they are both bonded form a five member heterocyclyl ring optionally substituted with two halogen atoms.

Some embodiments of the present invention pertain to compounds of Formula (IIc) and pharmaceutically acceptable salts, solvates and hydrates thereof:

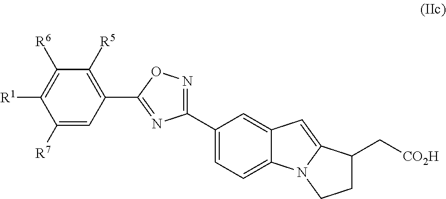

wherein:

R¹ is selected from the group consisting of H, C₁-C₆ alkoxy, C₁-C₆ haloalkoxy, halogen and hydroxyl;

R⁵ is H;

R⁶ is selected from the group consisting of C₁-C₆ alkoxy, cyano, C₁-C₆ haloalkoxy, C₁-C₆ haloalkyl, halogen and heteroaryl;

and

R⁷ is selected from the group consisting of H, C₁-C₆ alkoxy, cyano, C₁-C₆ haloalkoxy, C₁-C₆ haloalkyl, halogen and heteroaryl.

Some embodiments of the present invention pertain to compounds of Formula (IIc) and pharmaceutically acceptable salts, solvates and hydrates thereof:

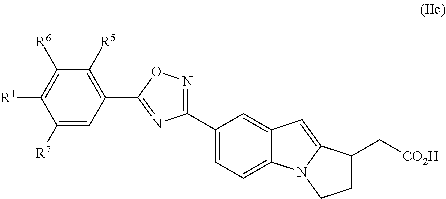

wherein:

R¹ is selected from the group consisting of H, methoxy, isopropoxy, trifluoromethoxy, 2,2,2-trifluoroethoxy, 1,1,1-trifluoropropan-2-yloxy, fluoro and hydroxyl;

R⁵ is H;

R⁶ is selected from the group consisting of cyano, trifluoromethoxy, difluoromethoxy, trifluoromethyl, bromo and pyridin-3-yl; and

R⁷ is selected from the group consisting of H, methoxy, cyano; trifluoromethoxy, trifluoromethyl and tetrazol-5-yl; or

R¹ and R⁶ together with the atoms to which they are both bonded form a benzo[1,3]-dioxole group optionally substituted with two fluorine atoms; or

R⁵ and R⁶ together with the atoms to which they are both bonded form a benzo[1,3]-dioxole group substituted with two fluorine atoms.

Some embodiments of the present invention pertain to compounds of Formula (IIc) and pharmaceutically acceptable salts, solvates and hydrates thereof:

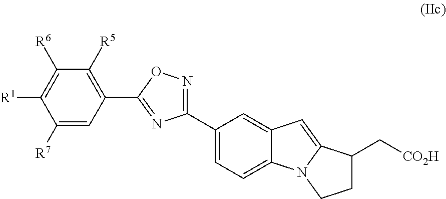

wherein:

R¹ is selected from the group consisting of H, methoxy, isopropoxy, trifluoromethoxy, 2,2,2-trifluoroethoxy, 1,1,1-trifluoropropan-2-yloxy, fluoro and hydroxyl;

R⁵ is H;

R⁶ is selected from the group consisting of cyano, trifluoromethoxy, difluoromethoxy, trifluoromethyl, bromo and pyridin-3-yl; and

R⁷ is selected from the group consisting of H, methoxy, cyano, trifluoromethoxy, trifluoromethyl and tetrazol-5-yl.

Some embodiments of the present invention pertain to compounds of Formula (IIe) and pharmaceutically acceptable salts, solvates and hydrates thereof:

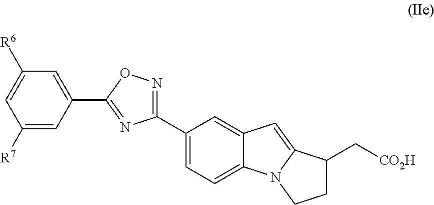

wherein:

R⁶ is selected from the group consisting of cyano, C₁-C₆ haloalkoxy, C₁-C₆ haloalkyl, halogen and heteroaryl;

and

R⁷ is selected from the group consisting of H, C₁-C₆ alkoxy, cyano, C₁-C₆ haloalkoxy, C₁-C₆ haloalkyl and heteroaryl.

Some embodiments of the present invention pertain to compounds of Formula (IIe) and pharmaceutically acceptable salts, solvates and hydrates thereof:

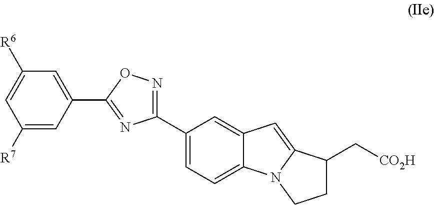

wherein:

R⁶ is selected from the group consisting of cyano, trifluoromethoxy, trifluoromethyl, bromo and pyridin-3-yl; and

R⁷ is selected from the group consisting of H, methoxy, cyano, trifluoromethoxy and trifluoromethyl.

Some embodiments of the present invention pertain to compounds of Formula (IIg) and pharmaceutically acceptable salts, solvates and hydrates thereof:

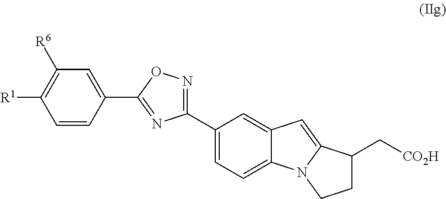

wherein:

R¹ is selected from the group consisting of H, C₁-C₆ alkoxy, C₁-C₆ haloalkoxy, halogen and hydroxyl; and

R⁶ is cyano or C₁-C₆ haloalkoxy.

Some embodiments of the present invention pertain to compounds of Formula (IIg) and pharmaceutically acceptable salts, solvates and hydrates thereof:

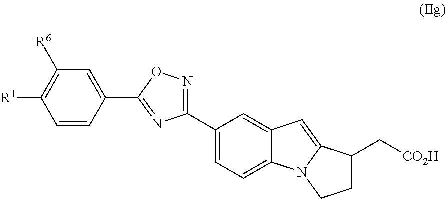

wherein:

R¹ is selected from the group consisting of H, methoxy, isopropoxy, trifluoromethoxy, 2,2,2-trifluoroethoxy, 1,1,1-trifluoropropan-2-yloxy, fluoro and hydroxyl; and

R⁶ is cyano or difluoromethoxy.

Some embodiments of the present invention pertain to compounds of Formula (IIa) and pharmaceutically acceptable salts, solvates and hydrates thereof:

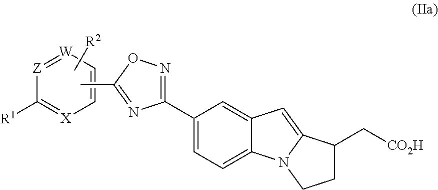

wherein:

W is N or CR⁵;

Z is N or CR⁶;

X is N or CR⁷; provided that at least one of W, Z and X is N, and provided that W, Z and X are not all N;

R¹ is selected from the group consisting of H, C₁-C₆ alkoxy, C₁-C₆ alkyl, C₁-C₆ alkylcarboxamide, cyano, C₁-C₆ haloalkyl, halogen, heteroaryl and heterocyclyl;

R² is selected from the group consisting of H, C₁-C₆ alkoxy and C₁-C₆ alkyl;

R⁵ is selected from the group consisting of H, C₁-C₆ alkyl, C₁-C₆ haloalkyl and hydroxyl;

R⁶ is selected from the group consisting of H, C₁-C₆ alkyl, and heteroaryl; and

R⁷ is H.

Some embodiments of the present invention pertain to compounds of Formula (IIa) and pharmaceutically acceptable salts, solvates and hydrates thereof:

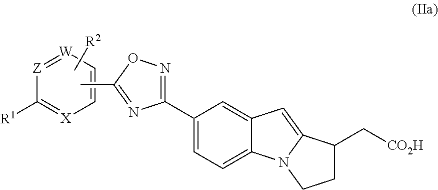

wherein:

W is N or CR⁵;

Z is N or CR⁶;

X is N or CR⁷; provided that at least one of W, Z and X is N, and provided that W, Z and X are not all N;

R¹ is selected from the group consisting of H, methoxy, methyl, acetamido, cyano, trifluoromethyl, chloro, pyrazol-1-yl and morpholino;

R² is selected from the group consisting of H, methoxy and methyl;

R⁵ is selected from the group consisting of H, methyl, trifluoromethyl and hydroxyl;

R⁶ is selected from the group consisting of H, methyl and thiophen-2-yl; and

R⁷ is H.

Some embodiments of the present invention pertain to compounds of Formula (IIi) and pharmaceutically acceptable salts, solvates and hydrates thereof:

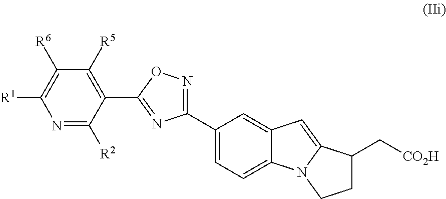

wherein:

R¹ is selected from the group consisting of H, C₁-C₆ alkoxy, C₁-C₆ alkyl, C₁-C₆ alkylcarboxamide, cyano, C₁-C₆ haloalkyl, heteroaryl and heterocyclyl;

R² is selected from the group consisting of H, C₁-C₆ alkoxy and C₁-C₆ alkyl;

R⁵ is selected from the group consisting of H, C₁-C₆ alkyl, C₁-C₆ haloalkyl and hydroxyl; and

R⁶ is selected from the group consisting of H, C₁-C₆ alkyl, and heteroaryl.

Some embodiments of the present invention pertain to compounds of Formula (IIi) and pharmaceutically acceptable salts, solvates and hydrates thereof:

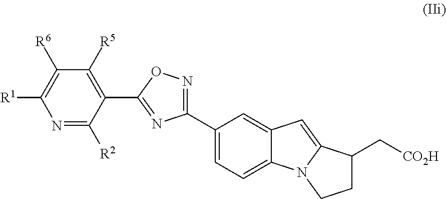

wherein:

R¹ is selected from the group consisting of H, methoxy, methyl, acetamido, cyano, trifluoromethyl, pyrazol-1-yl and morpholino;

R² is selected from the group consisting of H, methoxy and methyl;

R⁵ is selected from the group consisting of H, methyl, trifluoromethyl and hydroxyl; and

R⁶ is selected from the group consisting of H, methyl and thiophen-2-yl.

### C(1) Ring Carbon Stereochemistry

Compounds of the present invention contain the fused tricyclic system referred to as 2,3-dihydro-1H-pyrrolo[1,2-a]indole. Present on one of the rings is either a —CO₂H group (n=0) or a —CH₂CO₂H group (n=1). The ring carbon to which the carboxylic acid or acetic acid group is bonded is labeled as the C(1) ring carbon by convention. It is understood that the stereochemistry for the C(1) ring carbon contained in the 2,3-dihydro-1H-pyrrolo[1,2-a]indole ring system can be either R or S.

A. C(1) Ring Carbon “R” Stereochemistry

In some embodiments, the stereochemistry for the C(1) ring carbon is R.

Some embodiments of the present invention pertain to compounds of Formula (IIk) and pharmaceutically acceptable salts, solvates and hydrates thereof:

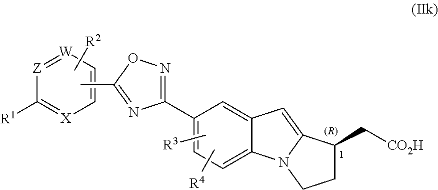

wherein each variable in Formula (IIk) has the same meaning as described herein, supra and infra.

Some embodiments of the present invention pertain to compounds of Formula (IIm) and pharmaceutically acceptable salts, solvates and hydrates thereof:

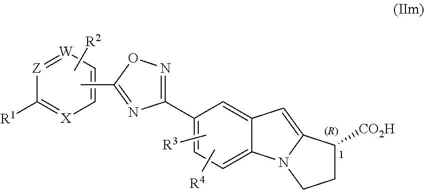

wherein each variable in Formula (IIm) has the same meaning as described herein, supra and infra.

Some embodiments of the present invention include every combination of one or more compounds selected from the following group: (R)-2-(7-(5-(3,5-bis(trifluoromethyl)phenyl)-1,2,4-oxadiazol-3-yl)-2,3-dihydro-1H-pyrrolo[1,2-a]indol-1-yl)acetic acid; (R)-2-(7-(5-(3-cyano-5-(trifluoromethoxy)phenyl)-1,2,4-oxadiazol-3-yl)-2,3-dihydro-1H-pyrrolo[1,2-a]indol-1-yl)acetic acid; (R)-2-(7-(5-(6-cyanopyridin-3-yl)-1,2,4-oxadiazol-3-yl)-2,3-dihydro-1H-pyrrolo[1,2-a]indol-1-yl)acetic acid; (R)-2-(7-(5-(3-cyano-5-methoxyphenyl)-1,2,4-oxadiazol-3-yl)-2,3-dihydro-1H-pyrrolo[1,2-a]indol-1-yl)acetic acid; (R)-2-(7-(5-(3-cyano-4-isopropoxyphenyl)-1,2,4-oxadiazol-3-yl)-2,3-dihydro-1H-pyrrolo[1,2-a]indol-1-yl)acetic acid; (R)-2-(7-(5-(benzo[d][1,3]dioxol-5-yl)-1,2,4-oxadiazol-3-yl)-2,3-dihydro-1H-pyrrolo[1,2-a]indol-1-yl)acetic acid; (R)-2-(7-(5-(2,6-dimethoxypyridin-3-yl)-1,2,4-oxadiazol-3-yl)-2,3-dihydro-1H-pyrrolo[1,2-a]indol-1-yl)acetic acid; (R)-2-(7-(5-(2,2-difluorobenzo[d][1,3]dioxol-5-yl)-1,2,4-oxadiazol-3-yl)-2,3-dihydro-1H-pyrrolo [1,2-a]indol-1-yl)acetic acid; (R)-2-(7-(5-(3-(1H-tetrazol-5-yl)-5-(trifluoromethoxy)phenyl)-1,2,4-oxadiazol-3-yl)-2,3-dihydro-1H-pyrrolo[1,2-a]indol-1-yl)acetic acid; (R)-2-(7-(5-(4-methylpyridin-3-yl)-1,2,4-oxadiazol-3-yl)-2,3-dihydro-1H-pyrrolo[1,2-a]indol-1-yl)acetic acid; (R)-2-(7-(5-(4-(trifluoromethyl)pyridin-3-yl)-1,2,4-oxadiazol-3-yl)-2,3-dihydro-1H-pyrrolo[1,2-a]indol-1-yl)acetic acid; (R)-2-(7-(5-(6-methylpyridin-3-yl)-1,2,4-oxadiazol-3-yl)-2,3-dihydro-1H-pyrrolo[1,2-a]indol-1-yl)acetic acid; (R)-2-(7-(5-(3-hydroxypyridin-2-yl)-1,2,4-oxadiazol-3-yl)-2,3-dihydro-1H-pyrrolo[1,2-a]indol-1-yl)acetic acid; (R)-2-(7-(5-(5-methylpyridin-3-yl)-1,2,4-oxadiazol-3-yl)-2,3-dihydro-1H-pyrrolo[1,2-a]indol-1-yl)acetic acid; (R)-2-(7-(5-(3-cyano-4-(2,2,2-trifluoroethoxy)phenyl)-1,2,4-oxadiazol-3-yl)-2,3-dihydro-1H-pyrrolo[1,2-a]indol-1-yl)acetic acid; (R)-2-(7-(5-(4-methylpyridin-2-yl)-1,2,4-oxadiazol-3-yl)-2,3-dihydro-1H-pyrrolo[1,2-a]indol-1-yl)acetic acid; (R)-2-(7-(5-(6-(1H-pyrazol-1-yl)pyridin-3-yl)-1,2,4-oxadiazol-3-yl)-2,3-dihydro-1H-pyrrolo[1,2-a]indol-1-yl)acetic acid; (R)-2-(7-(5-(3-bromo-5-(trifluoromethoxy)phenyl)-1,2,4-oxadiazol-3-yl)-2,3-dihydro-1H-pyrrolo[1,2-a]indol-1-yl)acetic acid; (R)-2-(7-(5-(3-(trifluoromethoxy)phenyl)-1,2,4-oxadiazol-3-yl)-2,3-dihydro-1H-pyrrolo[1,2-a]indol-1-yl)acetic acid; (R)-2-(7-(5-(3-cyanophenyl)-1,2,4-oxadiazol-3-yl)-2,3-dihydro-1H-pyrrolo[1,2-a]indol-1-yl)acetic acid; (R)-2-(7-(5-(2-methylpyridin-3-yl)-1,2,4-oxadiazol-3-yl)-2,3-dihydro-1H-pyrrolo[1,2-a]indol-1-yl)acetic acid; (R)-2-(7-(5-(5-methylpyrazin-2-yl)-1,2,4-oxadiazol-3-yl)-2,3-dihydro-1H-pyrrolo[1,2-a]indol-1-yl)acetic acid; (R)-2-(7-(5-(6-methylpyridin-2-yl)-1,2,4-oxadiazol-3-yl)-2,3-dihydro-1H-pyrrolo[1,2-a]indol-1-yl)acetic acid; (R)-2-(7-(5-(2,2-difluorobenzo[d][1,3]dioxol-4-yl)-1,2,4-oxadiazol-3-yl)-2,3-dihydro-1H-pyrrolo[1,2-a]indol-1-yl)acetic acid; (R)-2-(7-(5-(3-(difluoromethoxy)phenyl)-1,2,4-oxadiazol-3-yl)-2,3-dihydro-1H-pyrrolo[1,2-a]indol-1-yl)acetic acid; (R)-2-(7-(5-(6-acetamidopyridin-3-yl)-1,2,4-oxadiazol-3-yl)-2,3-dihydro-1H-pyrrolo[1,2-a]indol-1-yl)acetic acid; (R)-2-(7-(5-(6-morpholinopyridin-3-yl)-1,2,4-oxadiazol-3-yl)-2,3-dihydro-1H-pyrrolo[1,2-a]indol-1-yl)acetic acid; (R)-2-(7-(5-(5-(thiophen-2-yl)pyridin-3-yl)-1,2,4-oxadiazol-3-yl)-2,3-dihydro-1H-pyrrolo[1,2-a]indol-1-yl)acetic acid; (R)-2-(7-(5-(3-(pyridin-3-yl)-5-(trifluoromethoxy)phenyl)-1,2,4-oxadiazol-3-yl)-2,3-dihydro-1H-pyrrolo[1,2-a]indol-1-yl)acetic acid; (R)-2-(7-(5-(3-cyano-4-fluorophenyl)-1,2,4-oxadiazol-3-yl)-2,3-dihydro-1H-pyrrolo[1,2-a]indol-1-yl)acetic acid; (R)-2-(7-(5-(3-cyano-4-hydroxyphenyl)-1,2,4-oxadiazol-3-yl)-2,3-dihydro-1H-pyrrolo[1,2-a]indol-1-yl)acetic acid; (R)-2-(7-(5-(3-cyano-4-methoxyphenyl)-1,2,4-oxadiazol-3-yl)-2,3-dihydro-1H-pyrrolo[1,2-a]indol-1-yl)acetic acid; (R)-2-(7-(5-(3-cyano-4-(trifluoromethoxy)phenyl)-1,2,4-oxadiazol-3-yl)-2,3-dihydro-1H-pyrrolo[1,2-a]indol-1-yl)acetic acid; (R)-2-(7-(5-(6-(trifluoromethyl)pyridin-3-yl)-1,2,4-oxadiazol-3-yl)-2,3-dihydro-1H-pyrrolo[1,2-a]indol-1-yl)acetic acid; (R)-2-(7-(5-(2-chloro-6-methylpyridin-4-yl)-1,2,4-oxadiazol-3-yl)-2,3-dihydro-1H-pyrrolo[1,2-a]indol-1-yl)acetic acid; (R)-2-(7-(5-(3-cyano-4-(1,1,1-trifluoropropan-2-yloxy)phenyl)-1,2,4-oxadiazol-3-yl)-2,3-dihydro-1H-pyrrolo[1,2-a]indol-1-yl)acetic acid; (R)-2-(7-(5-(2,6-dichloropyridin-4-yl)-1,2,4-oxadiazol-3-yl)-2,3-dihydro-1H-pyrrolo[1,2-a]indol-1-yl)acetic acid; and (R)-2-(7-(5-(5-isopropoxypyrazin-2-yl)-1,2,4-oxadiazol-3-yl)-2,3-dihydro-1H-pyrrolo[1,2-a]indol-1-yl)acetic acid.

B. C(1) Ring Carbon “S” Stereochemistry

In some embodiments, the stereochemistry for the C(1) ring carbon is S.

Some embodiments of the present invention pertain to compounds of Formula (IIo) and pharmaceutically acceptable salts, solvates and hydrates thereof:

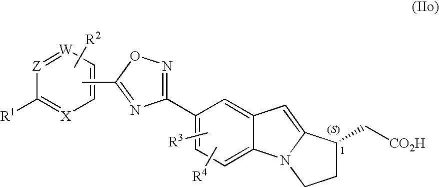

wherein each variable in Formula (IIo) has the same meaning as described herein, supra and infra.

Some embodiments of the present invention pertain to compounds of Formula (IIq) and pharmaceutically acceptable salts, solvates and hydrates thereof:

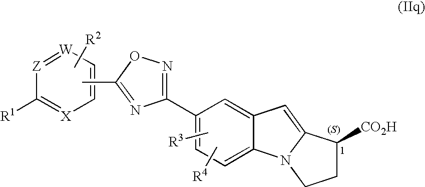

wherein each variable in Formula (IIq) has the same meaning as described herein, supra and infra.

Some embodiments of the present invention include every combination of one or more compounds selected from the following group: (S)-2-(7-(5-(3,5-bis(trifluoromethyl)phenyl)-1,2,4-oxadiazol-3-yl)-2,3-dihydro-1H-pyrrolo[1,2-a]indol-1-yl)acetic acid; (S)-2-(7-(5-(3-cyano-5-(trifluoromethoxy)phenyl)-1,2,4-oxadiazol-3-yl)-2,3-dihydro-1H-pyrrolo[1,2-a]indol-1-yl)acetic acid; (S)-2-(7-(5-(6-cyanopyridin-3-yl)-1,2,4-oxadiazol-3-yl)-2,3-dihydro-1H-pyrrolo[1,2-a]indol-1-yl)acetic acid; (S)-2-(7-(5-(3-cyano-5-methoxyphenyl)-1,2,4-oxadiazol-3-yl)-2,3-dihydro-1H-pyrrolo[1,2-a]indol-1-yl)acetic acid; (S)-2-(7-(5-(3-cyano-4-isopropoxyphenyl)-1,2,4-oxadiazol-3-yl)-2,3-dihydro-1H-pyrrolo[1,2-a]indol-1-yl)acetic acid; (S)-2-(7-(5-(benzo[d][1,3]dioxol-5-yl)-1,2,4-oxadiazol-3-yl)-2,3-dihydro-1H-pyrrolo[1,2-a]indol-1-yl)acetic acid; (S)-2-(7-(5-(2,6-dimethoxypyridin-3-yl)-1,2,4-oxadiazol-3-yl)-2,3-dihydro-1H-pyrrolo[1,2-a]indol-1-yl)acetic acid; (S)-2-(7-(5-(2,2-difluorobenzo[d][1,3]dioxol-5-yl)-1,2,4-oxadiazol-3-yl)-2,3-dihydro-1H-pyrrolo[1,2-a]indol-1-yl)acetic acid; (S)-2-(7-(5-(3-(1H-tetrazol-5-yl)-5-(trifluoromethoxy)phenyl)-1,2,4-oxadiazol-3-yl)-2,3-dihydro-1H-pyrrolo[1,2-a]indol-1-yl)acetic acid; (S)-2-(7-(5-(4-methylpyridin-3-yl)-1,2,4-oxadiazol-3-yl)-2,3-dihydro-1H-pyrrolo[1,2-a]indol-1-yl)acetic acid; (S)-2-(7-(5-(4-(trifluoromethyl)pyridin-3-yl)-1,2,4-oxadiazol-3-yl)-2,3-dihydro-1H-pyrrolo[1,2-a]indol-1-yl)acetic acid; (S)-2-(7-(5-(6-methylpyridin-3-yl)-1,2,4-oxadiazol-3-yl)-2,3-dihydro-1H-pyrrolo[1,2-a]indol-1-yl)acetic acid; (S)-2-(7-(5-(3-hydroxypyridin-2-yl)-1,2,4-oxadiazol-3-yl)-2,3-dihydro-1H-pyrrolo[1,2-a]indol-1-yl)acetic acid; (S)-2-(7-(5-(5-methylpyridin-3-yl)-1,2,4-oxadiazol-3-yl)-2,3-dihydro-1H-pyrrolo[1,2-a]indol-1-yl)acetic acid; (S)-2-(7-(5-(3-cyano-4-(2,2,2-trifluoroethoxy)phenyl)-1,2,4-oxadiazol-3-yl)-2,3-dihydro-1H-pyrrolo[1,2-a]indol-1-yl)acetic acid; (S)-2-(7-(5-(4-methylpyridin-2-yl)-1,2,4-oxadiazol-3-yl)-2,3-dihydro-1H-pyrrolo[1,2-a]indol-1-yl)acetic acid; (S)-2-(7-(5-(6-(1H-pyrazol-1-yl)pyridin-3-yl)-1,2,4-oxadiazol-3-yl)-2,3-dihydro-1H-pyrrolo[1,2-a]indol-1-yl)acetic acid; (S)-2-(7-(5-(3-bromo-5-(trifluoromethoxy)phenyl)-1,2,4-oxadiazol-3-yl)-2,3-dihydro-1H-pyrrolo[1,2-a]indol-1-yl)acetic acid; (S)-2-(7-(5-(3-(trifluoromethoxy)phenyl)-1,2,4-oxadiazol-3-yl)-2,3-dihydro-1H-pyrrolo[1,2-a]indol-1-yl)acetic acid; (S)-2-(7-(5-(3-cyanophenyl)-1,2,4-oxadiazol-3-yl)-2,3-dihydro-1H-pyrrolo[1,2-a]indol-1-yl)acetic acid; (S)-2-(7-(5-(2-methylpyridin-3-yl)-1,2,4-oxadiazol-3-yl)-2,3-dihydro-1H-pyrrolo[1,2-a]indol-1-yl)acetic acid; (S)-2-(7-(5-(5-methylpyrazin-2-yl)-1,2,4-oxadiazol-3-yl)-2,3-dihydro-1H-pyrrolo[1,2-a]indol-1-yl)acetic acid; (S)-2-(7-(5-(6-methylpyridin-2-yl)-1,2,4-oxadiazol-3-yl)-2,3-dihydro-1H-pyrrolo[1,2-a]indol-1-yl)acetic acid; (S)-2-(7-(5-(2,2-difluorobenzo[d][1,3]dioxol-4-yl)-1,2,4-oxadiazol-3-yl)-2,3-dihydro-1H-pyrrolo[1,2-a]indol-1-yl)acetic acid; (S)-2-(7-(5-(3-(difluoromethoxy)phenyl)-1,2,4-oxadiazol-3-yl)-2,3-dihydro-1H-pyrrolo[1,2-a]indol-1-yl)acetic acid; (S)-2-(7-(5-(6-acetamidopyridin-3-yl)-1,2,4-oxadiazol-3-yl)-2,3-dihydro-1H-pyrrolo[1,2-a]indol-1-yl)acetic acid; (S)-2-(7-(5-(6-morpholinopyridin-3-yl)-1,2,4-oxadiazol-3-yl)-2,3-dihydro-1H-pyrrolo[1,2-a]indol-1-yl)acetic acid; (S)-2-(7-(5-(5-(thiophen-2-yl)pyridin-3-yl)-1,2,4-oxadiazol-3-yl)-2,3-dihydro-1H-pyrrolo[1,2-a]indol-1-yl)acetic acid; (S)-2-(7-(5-(3-(pyridin-3-yl)-5-(trifluoromethoxy)phenyl)-1,2,4-oxadiazol-3-yl)-2,3-dihydro-1H-pyrrolo[1,2-a]indol-1-yl)acetic acid; (S)-2-(7-(5-(3-cyano-4-fluorophenyl)-1,2,4-oxadiazol-3-yl)-2,3-dihydro-1H-pyrrolo[1,2-a]indol-1-yl)acetic acid; (S)-2-(7-(5-(3-cyano-4-hydroxyphenyl)-1,2,4-oxadiazol-3-yl)-2,3-dihydro-1H-pyrrolo[1,2-a]indol-1-yl)acetic acid; (S)-2-(7-(5-(3-cyano-4-methoxyphenyl)-1,2,4-oxadiazol-3-yl)-2,3-dihydro-1H-pyrrolo[1,2-a]indol-1-yl)acetic acid; (S)-2-(7-(5-(3-cyano-4-(trifluoromethoxy)phenyl)-1,2,4-oxadiazol-3-yl)-2,3-dihydro-1H-pyrrolo[1,2-a]indol-1-yl)acetic acid; (S)-2-(7-(5-(6-(trifluoromethyl)pyridin-3-yl)-1,2,4-oxadiazol-3-yl)-2,3-dihydro-1H-pyrrolo[1,2-a]indol-1-yl)acetic acid; (S)-2-(7-(5-(2-chloro-6-methylpyridin-4-yl)-1,2,4-oxadiazol-3-yl)-2,3-dihydro-1H-pyrrolo[1,2-a]indol-1-yl)acetic acid; (S)-2-(7-(5-(3-cyano-4-(1,1,1-trifluoropropan-2-yloxy)phenyl)-1,2,4-oxadiazol-3-yl)-2,3-dihydro-1H-pyrrolo[1,2-a]indol-1-yl)acetic acid; (S)-2-(7-(5-(2,6-dichloropyridin-4-yl)-1,2,4-oxadiazol-3-yl)-2,3-dihydro-1H-pyrrolo[1,2-a]indol-1-yl)acetic acid; and (S)-2-(7-(5-(5-isopropoxypyrazin-2-yl)-1,2,4-oxadiazol-3-yl)-2,3-dihydro-1H-pyrrolo[1,2-a]indol-1-yl)acetic acid.

### Esters and Prodrugs

One aspect of the present invention pertains to compounds of Formula (IIIa) as synthetic intermediates useful in the preparation of compounds of Formula (Ia) and/or prodrug useful for the delivery of compounds of Formula (Ia):

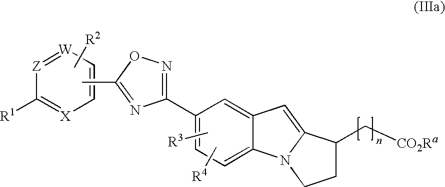

wherein:

n, R¹, R², R³, R⁴, W, X, and Z have the same definitions as described herein, supra and infra, and R^{a} is C₁-C₆ alkyl.

One aspect of the present invention pertains to compounds of Formula (IIIa).

In some embodiments, R^{a} is tert-butyl.

In some embodiments, R^{a} is methyl.

For brevity, it is appreciated that all of the embodiments described herein, supra and infra, that relate to the common variables shared between Compounds of Formula (Ia) and (IIIa) namely, n, R¹, R², R³ , R⁴, R⁵, W, X, and Z, apply to Compounds of Formula (IIIa) just as if they were each individually disclosed herewith with specific reference to Formula (IIIa).

One aspect of the present invention pertains to compounds of Formula (IIIa) as synthetic intermediates useful in the preparation of compounds of Formula (Ia).

One aspect of the present invention pertains to compounds of Formula (IIIa) as esters of compounds, described and shown herein, such as compounds in Table A, where R^{a} is tert-butyl.

One aspect of the present invention pertains to compounds of Formula (IIIa) as esters of compounds, described and shown herein, such as compounds in Table A, where R^{a} is methyl.

One aspect of the present invention pertains to compounds of Formula (IIIc) as prodrugs useful for the delivery of compounds of Formula (Ia).

One aspect of the present invention pertains to compounds of Formula (IIIa) useful as prodrugs of compounds of Formula (Ia).

Some embodiments of the present invention include every combination of one or more compounds selected from the following group shown in TABLE A.

TABLE A

| Cmpd | | |
|---|---|---|
| No. | Chemical Structure | Chemical Name |
| 1 | 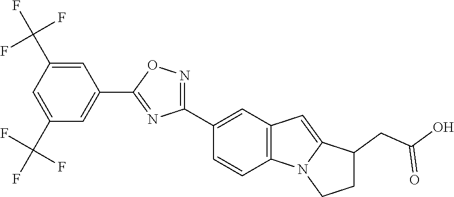 | 2-(7-(5-(3,5- bis(trifluoromethyl)phenyl)- 1,2,4-oxadiazol-3-yl)-2,3- dihydro-1H-pyrrolo[1,2- a]indol-1-yl)acetic acid |
| 2 | 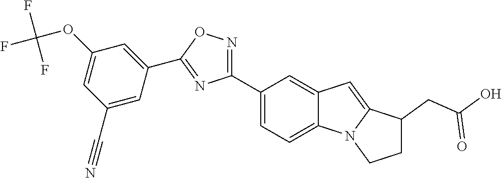 | 2-(7-(5-(3-cyano-5- (trifluoromethoxy)phenyl)- 1,2,4-oxadiazol-3-yl)-2,3- dihydro-1H-pyrrolo[1,2- a]indol-1-yl)acetic acid |
| 3 | 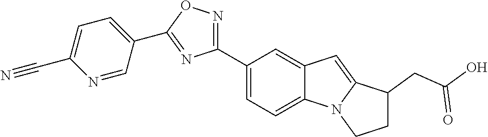 | 2-(7-(5-(6-cyanopyridin-3- yl)-1,2,4-oxadiazol-3-yl)- 2,3-dihydro-1H-pyrrolo[1,2- a]indol-1-yl)acetic acid |
| 4 | 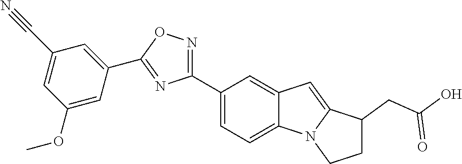 | 2-(7-(5-(3-cyano-5- methoxyphenyl)-1,2,4- oxadiazol-3-yl)-2,3-dihydro- 1H-pyrrolo[1,2-a]indol-1- yl)acetic acid |
| 5 | 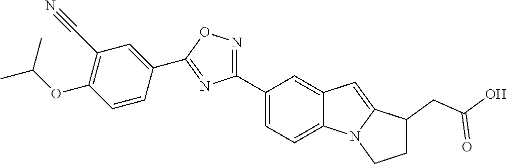 | 2-(7-(5-(3-cyano-4- isopropoxyphenyl)-1,2,4- oxadiazol-3-yl)-2,3-dihydro- 1H-pyrrolo[1,2-a]indol-1- yl)acetic acid |
| 6 | 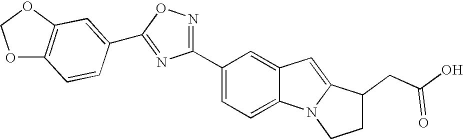 | 2-(7-(5- (benzo[d][1,3]dioxol-5-yl)- 1,2,4-oxadiazol-3-yl)-2,3- dihydro-1H-pyrrolo[1,2- a]indol-1-yl)acetic acid |
| 7 | 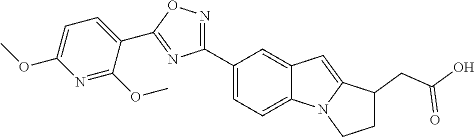 | 2-(7-(5-(2,6- dimethoxypyridin-3-yl)- 1,2,4-oxadiazol-3-yl)-2,3- dihydro-1H-pyrrolo[1,2- a]indol-1-yl)acetic acid |
| 8 | 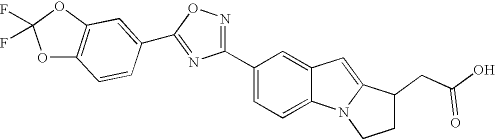 | 2-(7-(5-(2,2- difluorobenzo[d][1,3]dioxol- 5-yl)-1,2,4-oxadiazol-3-yl)- 2,3-dihydro-1H-pyrrolo[1,2- a]indol-1-yl)acetic acid |
| 9 | 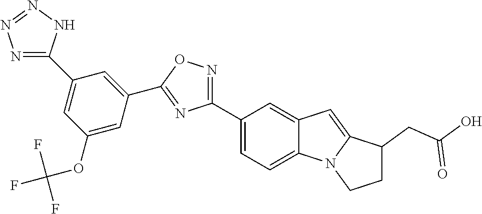 | 2-(7-(5-(3-(1H-tetrazol-5-yl)- 5-(trifluoromethoxy)phenyl)- 1,2,4-oxadiazol-3-yl)-2,3- dihydro-1H-pyrrolo[1,2- a]indol-1-yl)acetic acid |
| 10 | 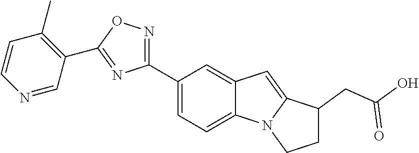 | 2-(7-(5-(4-methylpyridin-3- yl)-1,2,4-oxadiazol-3-yl)- 2,3-dihydro-1H-pyrrolo[1,2- a]indol-1-yl)acetic acid |
| 11 | 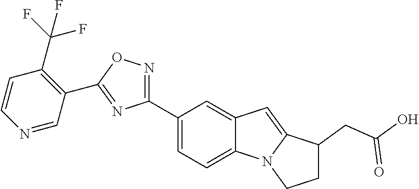 | 2-(7-(5-(4- (trifluoromethyl)pyridin-3- yl)-1,2,4-oxadiazol-3-yl)- 2,3-dihydro-1H-pyrrolo[1,2- a]indol-1-yl)acetic acid |
| 12 | 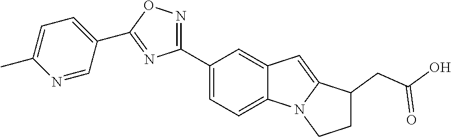 | 2-(7-(5-(6-methylpyridin-3- yl)-1,2,4-oxadiazol-3-yl)- 2,3-dihydro-1H-pyrrolo[1,2- a]indol-1-yl)acetic acid |
| 13 | 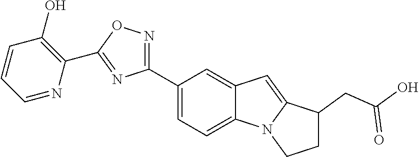 | 2-(7-(5-(3-hydroxypyridin- 2-yl)-1,2,4-oxadiazol-3-yl)- 2,3-dihydro-1H-pyrrolo[1,2- a]indol-1-yl)acetic acid |
| 14 | 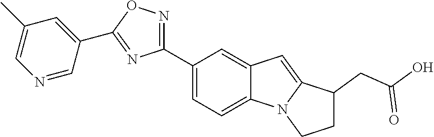 | 2-(7-(5-(5-methylpyridin-3- yl)-1,2,4-oxadiazol-3-yl)- 2,3-dihydro-1H-pyrrolo[1,2- a]indol-1-yl)acetic acid |
| 15 | 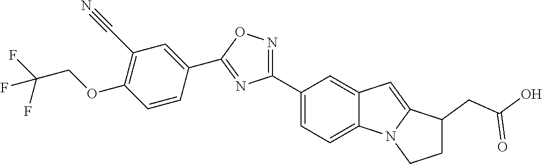 | 2-(7-(5-(3-cyano-4-(2,2,2- trifluoromethoxy)phenyl)- 1,2,4-oxadiazol-3-yl)-2,3- dihydro-1H-pyrrolo[1,2- a]indol-1-yl)acetic acid |
| 16 | 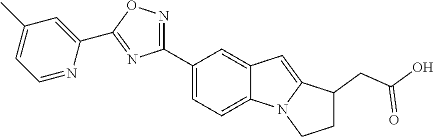 | 2-(7-(5-(4-methylpyridin-2- yl)-1,2,4-oxadiazol-3-yl)- 2,3-dihydro-1H-pyrrolo[1,2- a]indol-1-yl)acetic acid |
| 17 | 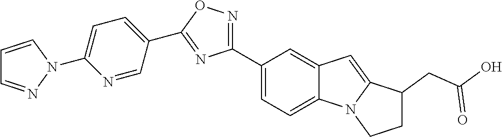 | 2-(7-(5-(6-(1H-pyrazol-1- yl)pyridin-3-yl)-1,2,4- oxadiazol-3-yl)-2,3-dihydro- 1H-pyrrolo[1,2-a]indol-1- yl)acetic acid |
| 18 | 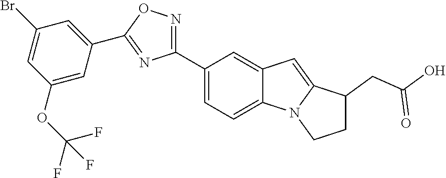 | 2-(7-(5-(3-bromo-5- (trifluoromethoxy)phenyl)- 1,2,4-oxadiazol-3-yl)-2,3- dihydro-1H-pyrrolo[1,2- a]indol-1-yl)acetic acid |
| 19 | 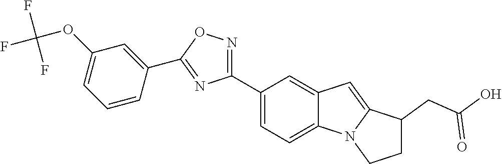 | 2-(7-(5-(3- (trifluoromethoxy)phenyl)- 1,2,4-oxadiazol-3-yl)-2,3- dihydro-1H-pyrrolo[1,2- a]indol-1-yl)acetic acid |
| 20 | 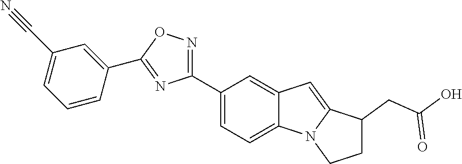 | 2-(7-(5-(3-cyanophenyl)- 1,2,4-oxadiazol-3-yl)-2,3- dihydro-1H-pyrrolo[1,2- a]indol-1-yl)acetic acid |
| 21 | 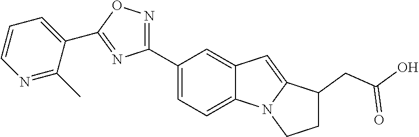 | 2-(7-(5-(2-methylpyridin-3- yl)-1,2,4-oxadiazol-3-yl)- 2,3-dihydro-1H-pyrrolo[1,2- a]indol-1-yl)acetic acid |
| 22 | 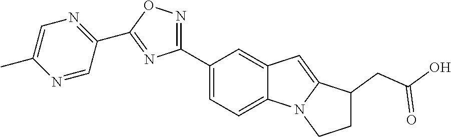 | 2-(7-(5-(5-methylpyrazin-2- yl)-1,2,4-oxadiazol-3-yl)- 2,3-dihydro-1H-pyrrrolo[1,2- a]indol-1-yl)acetic acid |
| 23 | 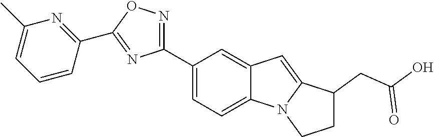 | 2-(7-(5-(6-methylpyridin-2- yl)-1,2,4-oxadiazol-3-yl)- 2,3-dihydro-1H-pyrrolo[1,2- a]indol-1-yl)acetic acid |
| 24 | 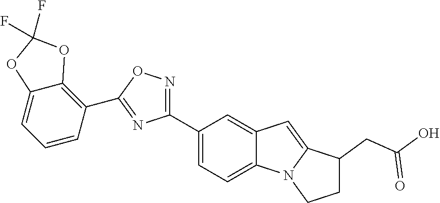 | 2-(7-(5-(2,2- difluorobenzo[d][1,3]dioxol- 4-yl)-1,2,4-oxadiazol-3-yl)- 2,3-dihydro-1H-pyrrolo[1,2- a]indol-1-yl)acetic acid |
| 25 | 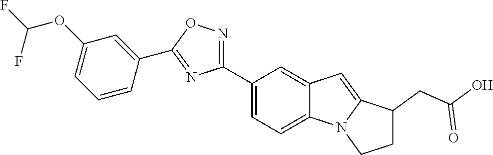 | 2-(7-(5-(3- (difluoromethoxy)phenyl)- 1,2,4-oxadiazol-3-yl)-2,3- dihydro-1H-pyrrolo[1,2- a]indol-1-yl)acetic acid |
| 26 | 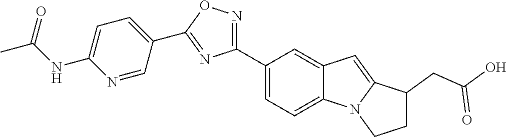 | 2-(7-(5-(6- acetamidopyridin-3-yl)- 1,2,4-oxadiazol-3-yl)-2,3- dihydro-1H-pyrrolo[1,2- a]indol-1-yl)acetic acid |
| 27 | 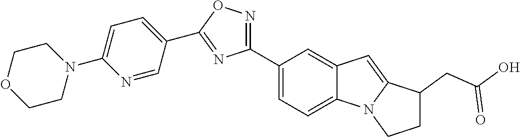 | 2-(7-(5-(6- morpholinopyridin-3-yl)- 1,2,4-oxadiazol-3-yl)-2,3- dihydro-1H-pyrrolo[1,2- a]indol-1-yl)acetic acid |
| 28 | 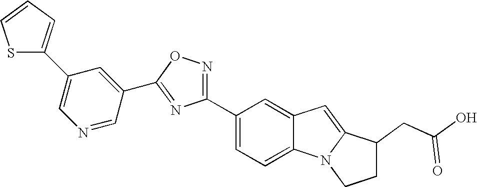 | 2-(7-(5-(5-(thiophen-2- yl)pyridin-3-yl)-1,2,4- oxadiazol-3-yl)-2,3-dihydro- 1H-pyrrolo[1,2-a]indol-1- yl)acetic acid |
| 29 | 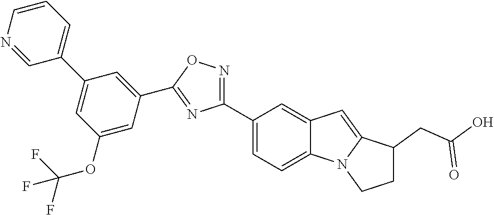 | 2-(7-(5-(3-(pyridin-3-yl)-5- (trifluoromethoxy)phenyl)- 1,2,4-oxadiazol-3-yl)-2,3- dihydro-1H-pyrrolo[1,2- a]indol-1-yl)acetic acid |
| 30 | 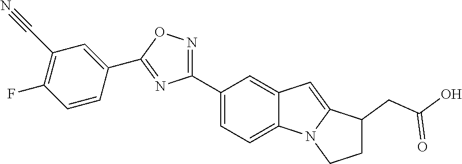 | 2-(7-(5-(3-cyano-4- fluorophenyl)-1,2,4- oxadiazol-3-yl)-2,3-dihydro- 1H-pyrrolo[1,2-a]indol-1- yl)acetic acid |
| 31 | 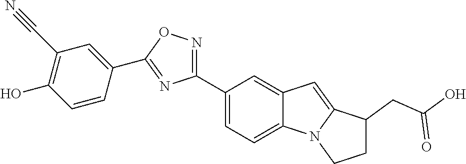 | 2-(7-(5-(3-cyano-4- hydroxyphenyl)-1,2,4- oxadiazol-3-yl)-2,3-dihydro- 1H-pyrrolo[1,2-a]indol-1- yl)acetic acid |
| 32 | 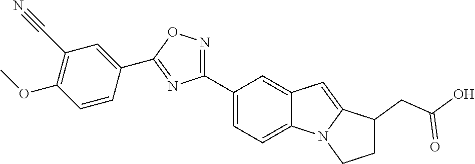 | 2-(7-(5-(3-cyano-4- methoxyphenyl)-1,2,4- oxadiazol-3-yl)-2,3-dihydro- 1H-pyrrolo[1,2-a]indol-1- yl)acetic acid |
| 33 | 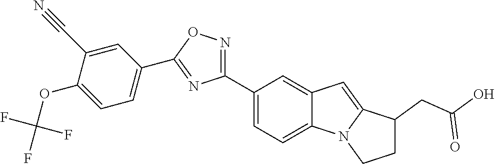 | 2-(7-(5-(3-cyano-4- (trifluoromethoxy)phenyl)- 1,2,4-oxadiazol-3-yl)-2,3- dihydro-1H-pyrrolo[1,2- a]indol-1-yl)acetic acid |
| 34 | 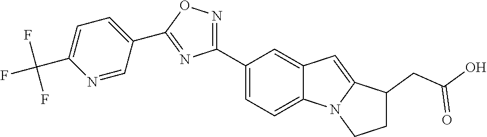 | 2-(7-(5-(6- (trifluoromethyl)pyridin-3- yl)-1,2,4-oxadiazol-3-yl)- 2,3-dihydro-1H-pyrrolo[1,2- a]indol-1-yl)acetic acid |
| 35 | 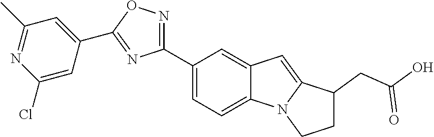 | 2-(7-(5-(2-chloro-6- methylpyridin-4-yl)-1,2,4- oxadiazol-3-yl)-2,3-dihydro- 1H-pyrrolo[1,2-a]indol-1- yl)acetic acid |
| 36 | 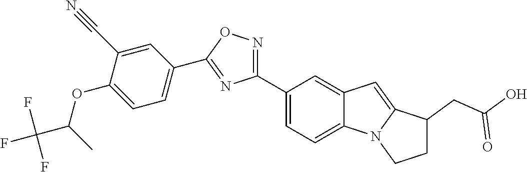 | 2-(7-(5-(3-cyano-4-(1,1,1- trifluoropropan-2- yloxy)phenyl)-1,2,4- oxadiazol-3-yl)-2,3-dihydro- 1H-pyrrolo[1,2-a]indol-1- yl)acetic acid |
| 37 | 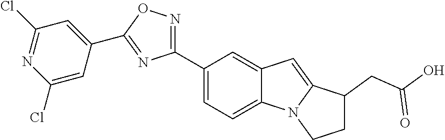 | 2-(7-(5-(2,6- dichloropyridin-4-yl)-1,2,4- oxadiazol-3-yl)-2,3-dihydro- 1H-pyrrolo[1,2-a]indol-1- yl)acetic acid |
| 38 | 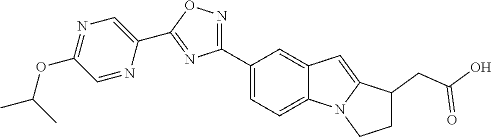 | 2-(7-(5-(5- isopropoxypyrazin-2-yl)- 1,2,4-oxadiazol-3-yl)-2,3- dihydro-1H-pyrrolo[1,2- a]indol-1-yl)acetic acid |

Additionally, individual compounds and chemical genera of the present invention, for example those compounds found in TABLE A including diastereomers and enantiomers thereof, encompass all pharmaceutically acceptable salts, solvates and particularly hydrates, thereof.

It is understood that the present invention embraces each diastereomer, each enantiomer and mixtures thereof of each compound and generic formula disclosed herein just as if they were each individually disclosed with the specific stereochemical designation for each chiral carbon. Separation of the individual isomers (such as, by chiral HPLC, recrystallization of diastereomeric mixtures and the like) or selective synthesis (such as, by enantiomeric selective syntheses and the like) of the individual isomers is accomplished by application of various methods which are well known to practitioners in the art.

The compounds of the Formula (Ia) of the present invention may be prepared according to relevant published literature procedures that are used by one skilled in the art. Exemplary reagents and procedures for these reactions appear hereinafter in the working Examples. Protection and deprotection may be carried out by procedures generally known in the art (see, for example, Greene, T. W. and Wuts, P. G. M., Protecting Groups in Organic Synthesis, 3^{rd} Edition, 1999 [Wiley]; incorporated herein by reference in its entirety).

### Pharmaceutical Compositions

A further aspect of the present invention pertains to pharmaceutical compositions comprising one or more compounds as described herein and one or more pharmaceutically acceptable carriers. Some embodiments pertain to pharmaceutical compositions comprising a compound of the present invention and a pharmaceutically acceptable carrier.

Some embodiments of the present invention include a method of producing a pharmaceutical composition comprising admixing at least one compound according to any of the compound embodiments disclosed herein and a pharmaceutically acceptable carrier.

Formulations may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tabletting lubricants and disintegrants may be used in tablets and capsules for oral administration. Liquid preparations for oral administration may be in the form of solutions, emulsions, aqueous or oily suspensions and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives and flavorings and colorants may be added to the liquid preparations. Parenteral dosage forms may be prepared by dissolving the compound of the invention in a suitable liquid vehicle and filter sterilizing the solution before filling and sealing an appropriate vial or ampule. These are just a few examples of the many appropriate methods well known in the art for preparing dosage forms.

A compound of the present invention can be formulated into pharmaceutical compositions using techniques well known to those in the art. Suitable pharmaceutically-acceptable carriers, outside those mentioned herein, are known in the art; for example, see Remington, The Science and Practice of Pharmacy, 20^{th} Edition, 2000, Lippincott Williams & Wilkins, (Editors: Gennaro et al.)

While it is possible that, for use in the prophylaxis or treatment, a compound of the invention may, in an alternative use, be administered as a raw or pure chemical, it is preferable however to present the compound or active ingredient as a pharmaceutical formulation or composition further comprising a pharmaceutically acceptable carrier.

The invention thus further provides pharmaceutical formulations comprising a compound of the invention or a pharmaceutically acceptable salt, solvate, hydrate or derivative thereof together with one or more pharmaceutically acceptable carriers thereof and/or prophylactic ingredients. The carrier(s) must be “acceptable” in the sense of being compatible with the other ingredients of the formulation and not overly deleterious to the recipient thereof.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation, insufflation or by a transdermal patch. Transdermal patches dispense a drug at a controlled rate by presenting the drug for absorption in an efficient manner with a minimum of degradation of the drug. Typically, transdermal patches comprise an impermeable backing layer, a single pressure sensitive adhesive and a removable protective layer with a release liner. One of ordinary skill in the art will understand and appreciate the techniques appropriate for manufacturing a desired efficacious transdermal patch based upon the needs of the artisan.

The compounds of the invention, together with a conventional adjuvant, carrier, or diluent, may thus be placed into the form of pharmaceutical formulations and unit dosages thereof and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

Compounds of the present invention or a salt, solvate, hydrate or physiologically functional derivative thereof can be used as active ingredients in pharmaceutical compositions, specifically as S1P1 receptor modulators. The term “active ingredient” is defined in the context of a “pharmaceutical composition” and is intended to mean a component of a pharmaceutical composition that provides the primary pharmacological effect, as opposed to an “inactive ingredient” which would generally be recognized as providing no pharmaceutical benefit.

The dose when using the compounds of the present invention can vary within wide limits and as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis is conducted or on whether further active compounds are administered in addition to the compounds of the present invention. Representative doses of the present invention include, but not limited to, about 0.001 mg to about 5000 mg, about 0.001 mg to about 2500 mg, about 0.001 mg to about 1000 mg, 0.001 mg to about 500 mg, 0.001 mg to about 250 mg, about 0.001 mg to 100 mg, about 0.001 mg to about 50 mg and about 0.001 mg to about 25 mg. Multiple doses may be administered during the day, especially when relatively large amounts are deemed to be needed, for example 2, 3 or 4 doses. Depending on the individual and as deemed appropriate from the patient's physician or caregiver it may be necessary to deviate upward or downward from the doses described herein.

The amount of active ingredient, or an active salt, solvate or hydrate or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, whether an acute or chronic disease state is being treated or prophylaxis is conducted or whether further active compounds are administered in addition to the compounds of the present invention and as part of a drug combination. The dosage regimen for treating a disease condition with the compounds and/or compositions of this invention is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods of this invention.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4 part administrations. If appropriate, depending on individual behavior, it may be necessary to deviate upward or downward from the daily dose indicated.

For preparing pharmaceutical compositions from the compounds of the present invention, the suitable pharmaceutically acceptable carrier can be either solid, liquid or a mixture of both. Solid form preparations include powders, tablets, pills, capsules, cachets, suppositories and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted to the desired shape and size.

The powders and tablets may contain varying percentage amounts of the active compound. A representative amount in a powder or tablet may contain from 0.5 to about 90 percent of the active compound; however, an artisan would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous formulations suitable for oral use can be prepared by dissolving or suspending the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well-known suspending agents.

Also included are solid form preparations which are intended to be converted, shortly before use, to liquid form preparations for oral administration. Such liquid forms include solutions, suspensions and emulsions. These preparations may contain, in addition to the active component, colorants, flavors, stabilizers, buffers, artificial and natural sweeteners, dispersants, thickeners, solubilizing agents and the like.

For topical administration to the epidermis the compounds according to the invention may be formulated as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the latter case of a dropper or pipette, this may be achieved by the patient administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the compounds of the present invention or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds of the present invention as an aerosol can be prepared by processes well known to the person skilled in the art. For their preparation, for example, solutions or dispersions of the compounds of the present invention or a pharmaceutically acceptable salt, solvate, hydrate or derivative thereof in water, water/alcohol mixtures or suitable saline solutions can be employed using customary additives, for example benzyl alcohol or other suitable preservatives, absorption enhancers for increasing the bioavailability, solubilizers, dispersants and others and, if appropriate, customary propellants, for example include carbon dioxide, CFCs, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the compound will generally have a small particle size for example of the order of 10 microns or less. Such a particle size may be obtained by means known in the art, for example by micronization. When desired, formulations adapted to give sustained release of the active ingredient may be employed.

Alternatively the active ingredients may be provided in the form of a dry powder, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfiric, tartaric, oxalic, p-toluenesulfonic and the like, such as those pharmaceutically acceptable salts listed in Journal of Pharmaceutical Sciences, 66:1-19 (1977), incorporated herein by reference in its entirety.

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds of this invention may form solvates with standard low molecular weight solvents using methods known to the skilled artisan.

Compounds of the present invention can be converted to “pro-drugs.” The term “pro-drugs” refers to compounds that have been modified with specific chemical groups known in the art and when administered into an individual undergo biotransformation to give the parent compound. Pro-drugs can thus be viewed as compounds of the invention containing one or more specialized non-toxic protective groups used in a transient mariner to alter or to eliminate a property of the compound. In one general aspect, the “pro-drug” approach is utilized to facilitate oral absorption. A thorough discussion is provided in T. Higuchi and V. Stella, Pro-drugs as Novel Delivery Systems Vol. 14 of the A.C.S. Symposium Series; and in Bioreversible Carriers in Drug Design, ed. Edward B. Roche, American Pharmaceutical Association and Pergamon Press, 1987, both of which are hereby incorporated by reference in their entirety.

Some embodiments of the present invention include a method of producing a pharmaceutical composition for “combination-therapy” comprising admixing at least one compound according to any of the compound embodiments disclosed herein, together with at least one known pharmaceutical agent as described herein and a pharmaceutically acceptable carrier.

It is noted that when the S1P1 receptor agonists are utilized as active ingredients in a pharmaceutical composition, these are not intended for use only in humans, but in other non-human mammals as well. Indeed, recent advances in the area of animal health-care mandate that consideration be given for the use of active agents, such as S1P1 receptor agonists, for the treatment of a S1P1 receptor associated disease or disorder in companionship animals (e.g., cats, dogs, etc.) and in livestock animals (e.g., cows, chickens, fish, etc.) Those of ordinary skill in the art are readily credited with understanding the utility of such compounds in such settings.

### Hydrates and Solvates

It is understood that when the phrase “pharmaceutically acceptable salts, solvates and hydrates” is used when referring to a particular formula herein, it is intended to embrace solvates and/or hydrates of compounds of the particular formula, pharmaceutically acceptable salts of compounds of the particular formula as well as solvates and/or hydrates of pharmaceutically acceptable salts of compounds of the particular formula.

The compounds of the present invention can be administrated in a wide variety of oral and parenteral dosage forms. It will be apparent to those skilled in the art that the following dosage forms may comprise, as the active component, either a compound of the invention or a pharmaceutically acceptable salt or as a solvate or hydrate thereof. Moreover, various hydrates and solvates of the compounds of the invention and their salts will find use as intermediates in the manufacture of pharmaceutical compositions. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Brittan, Vol. 95, Marcel Dekker, Inc., New York, 1999, incorporated herein by reference in its entirety. Accordingly, one aspect of the present invention pertains to hydrates and solvates of compounds of Formula (Ia) or Formula (IIa) and/or their pharmaceutical acceptable salts, as described herein, that can be isolated and characterized by methods known in the art, such as, thermogravimetric analysis (TGA), TGA-mass spectroscopy, TGA-Infrared spectroscopy, powder X-ray diffraction (XRPD), Karl Fisher titration, high resolution X-ray diffraction, and the like. There are several commercial entities that provide quick and efficient services for identifying solvates and hydrates on a routine basis. Example companies offering these services include Wilmington PharmaTech (Wilmington, Del.), Avantium Technologies (Amsterdam) and Aptuit (Greenwich, Conn.).

### Other Utilities

Another object of the present invention relates to radio-labeled compounds of the present invention that would be useful not only in radio-imaging but also in assays, both in vitro and in vivo, for localizing and quantitating the S1P1 receptor in tissue samples (including human) and for identifying S1P1 receptor ligands by inhibition binding of a radio-labeled compound. It is a further object of this invention to develop novel S1P1 receptor assays of which comprise such radio-labeled compounds.

The present invention embraces isotopically-labeled compounds of the present invention. Isotopically or radio-labeled compounds are those which are identical to compounds disclosed herein, but for the fact that one or more atoms are replaced or substituted by an atom having an atomic mass or mass number different from the atomic mass or mass number most commonly found in nature. Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁷⁵Br, ⁷⁶Br, ⁷⁷Br, ⁸²Br, ¹²³I, ¹²⁴I, ¹²⁵I and ¹³¹I. The radionuclide that is incorporated in the instant radio-labeled compounds will depend on the specific application of that radio-labeled compound. For example, for in vitro S1P1 receptor labeling and competition assays, compounds that incorporate ³H, ¹⁴C, ⁸²Br, ¹²⁵I, ¹³¹I or ³⁵S will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled ” or “labeled compound” is a compound of Formula (Ia), (Ic), (Ie), etc that has incorporated at least one radionuclide; in some embodiments the radionuclide is selected from the group consisting of ³H, ¹⁴C, ¹²⁵I , ³⁵S and ⁸²Br.

Certain isotopically-labeled compounds of the present invention are useful in compound and/or substrate tissue distribution assays. In some embodiments the radionuclide ³H and/or ¹⁴C isotopes are useful in these studies. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present invention can generally be prepared by following procedures analogous to those disclosed in the Drawings and Examples infra, by substituting an isotopically labeled reagent for a non-isotopically labeled reagent. Other synthetic methods that are useful are discussed infra. Moreover, it should be understood that all of the atoms represented in the compounds of the invention can be either the most commonly occurring isotope of such atoms or the scarcer radio-isotope or nonradioactive isotope.

Synthetic methods for incorporating radio-isotopes into organic compounds are applicable to compounds of the invention and are well known in the art. These synthetic methods, for example, incorporating activity levels of tritium into target molecules, areas follows:

A. Catalytic Reduction with Tritium Gas: This procedure normally yields high specific activity products and requires halogenated or unsaturated precursors.

B. Reduction with Sodium Borohydride [³H]: This procedure is rather inexpensive and requires precursors containing reducible functional groups such as aldehydes, ketones, lactones, esters and the like.

C. Reduction with Lithium Aluminum Hydride [³H]: This procedure offers products at almost theoretical specific activities. It also requires precursors containing reducible functional groups such as aldehydes, ketones, lactones, esters and the like.

D. Tritium Gas Exposure Labeling: This procedure involves exposing precursors containing exchangeable protons to tritium gas in the presence of a suitable catalyst.

E. N-Methylation using Methyl Iodide [³H]: This procedure is usually employed to prepare O-methyl or N-methyl (³H) products by treating appropriate precursors with high specific activity methyl iodide (³H). This method in general allows for higher specific activity, such as for example, about 70-90 Ci/mmol.

Synthetic methods for incorporating activity levels of ¹²⁵I into target molecules include:

A. Sandmeyer and like reactions: This procedure transforms an aryl amine or a heteroaryl amine into a diazonium salt, such as a diazonium tetrafluoroborate salt and subsequently to ¹²⁵I labeled compound using Na¹²⁵I. A represented procedure was reported by Zhu, G-D. and co-workers in J. Org. Chem., 2002, 67, 943-948.

B. Ortho ¹²⁵Iodination of phenols: This procedure allows for the incorporation of ¹²⁵I at the ortho position of a phenol as reported by Collier, T. L. and co-workers in J. Labelled Compd. Radiopharm., 1999, 42, S264-S266.

C. Aryl and heteroaryl bromide exchange with ¹²⁵I: This method is generally a two step process. The first step is the conversion of the aryl or heteroaryl bromide to the corresponding tri-alkyltin intermediate using for example, a Pd catalyzed reaction [i.e. Pd(Ph₃P)₄] or through an aryl or heteroaryl lithium, in the presence of a tri-alkyltinhalide or hexaalkylditin [e.g., (CH₃)₃SnSn(CH₃)₃]. A representative procedure was reported by Le Bas, M.-D. and co-workers in J. Labelled Compd. Radiopharm. 2001, 44, S280-S282.

A radiolabeled S1P1 receptor compound of Formula (Ia) can be used in a screening assay to identify/evaluate compounds. In general terms, a newly synthesized or identified compound (i.e., test compound) can be evaluated for its ability to reduce binding of the “radio-labeled compound of Formula (Ia)” to the S1P1 receptor. Accordingly, the ability of a test compound to compete with the “radio-labeled compound of Formula (Ia)” for the binding to the S1P1 receptor directly correlates to its binding affinity.

The labeled compounds of the present invention bind to the S1P1 receptor. In one embodiment the labeled compound has an IC₅₀ less than about 500 μM, in another embodiment the labeled compound has an IC₅₀ less than about 100 μM, in yet another embodiment the labeled compound has an IC₅₀ less than about 10 μM, in yet another embodiment the labeled compound has an IC₅₀ less than about 1 μM and in still yet another embodiment the labeled inhibitor has an IC₅₀ less than about 0.1 μM.

Other uses of the disclosed receptors and methods will become apparent to those in the art based upon, inter alia, a review of this disclosure.

As will be recognized, the steps of the methods of the present invention need not be performed any particular number of times or in any particular sequence. Additional objects, advantages and novel features of this invention will become apparent to those skilled in the art upon examination of the following examples thereof, which are intended to be illustrative and not intended to be limiting.

### EXAMPLES

### Example 1

### Syntheses of Compounds of the Present Invention

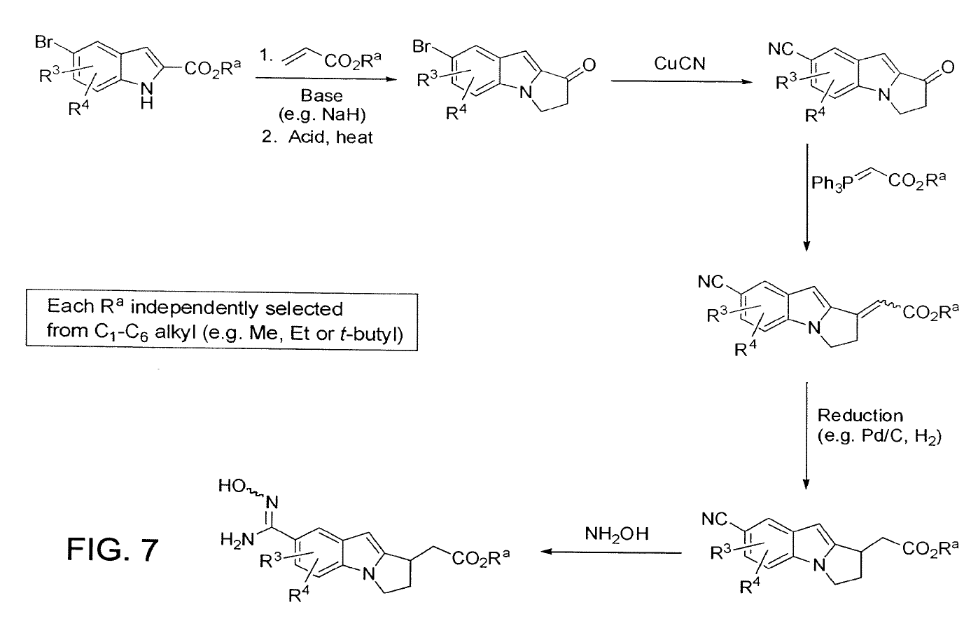

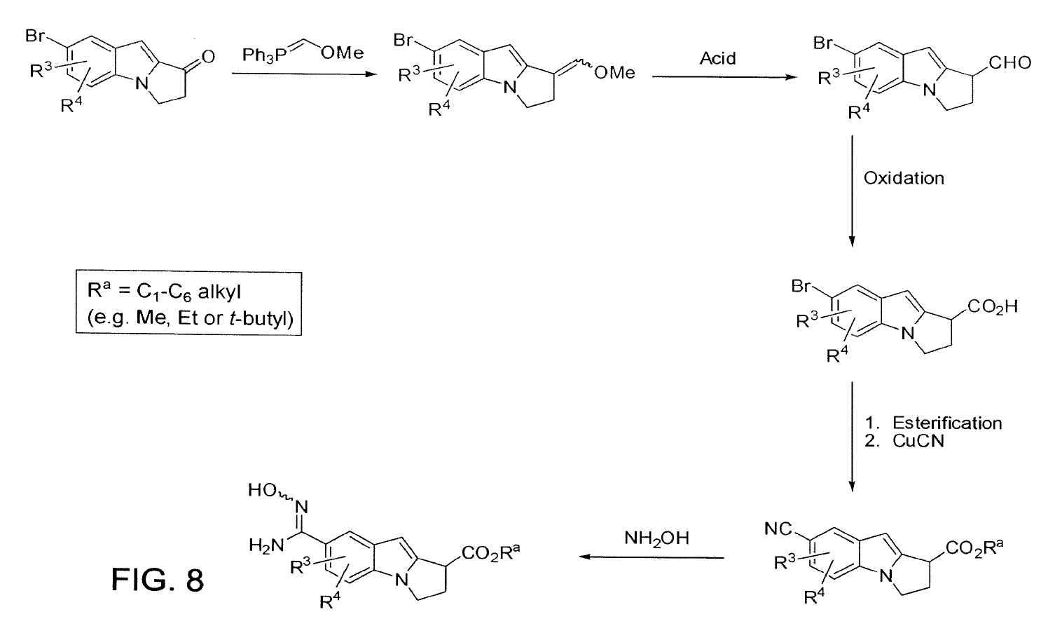

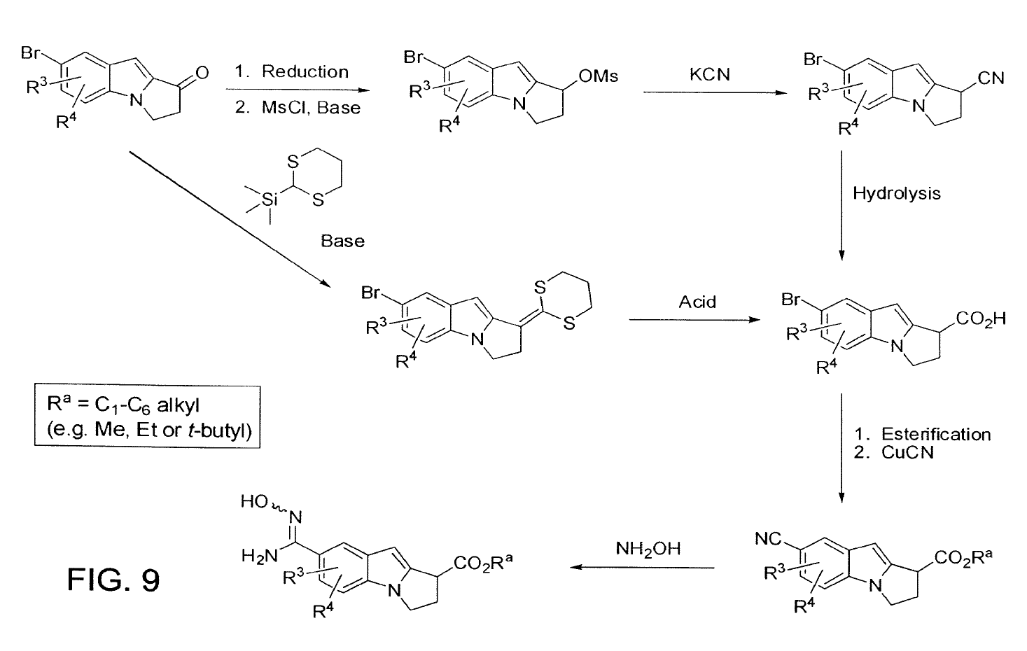

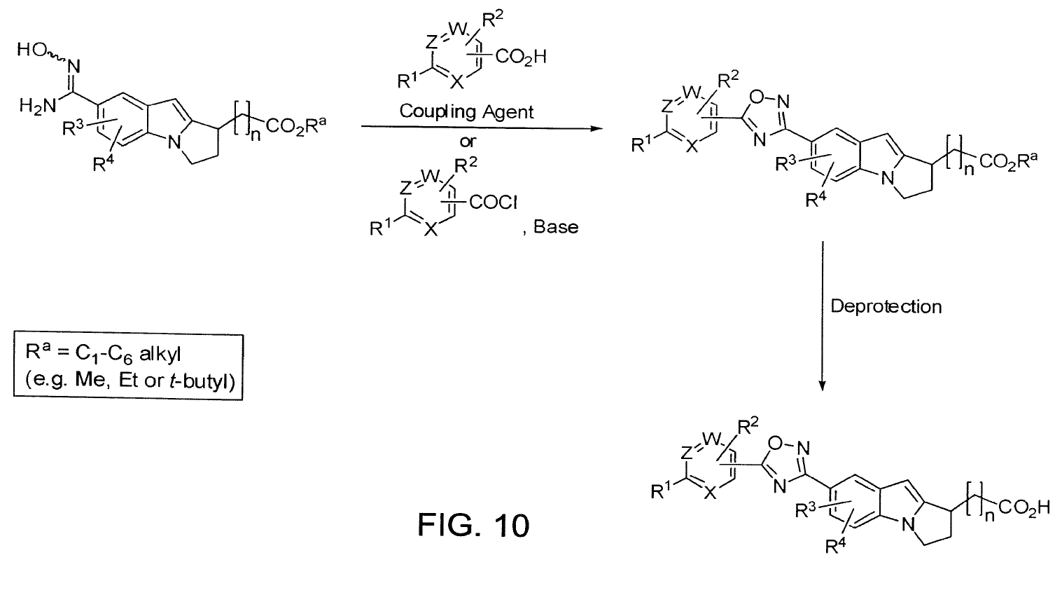

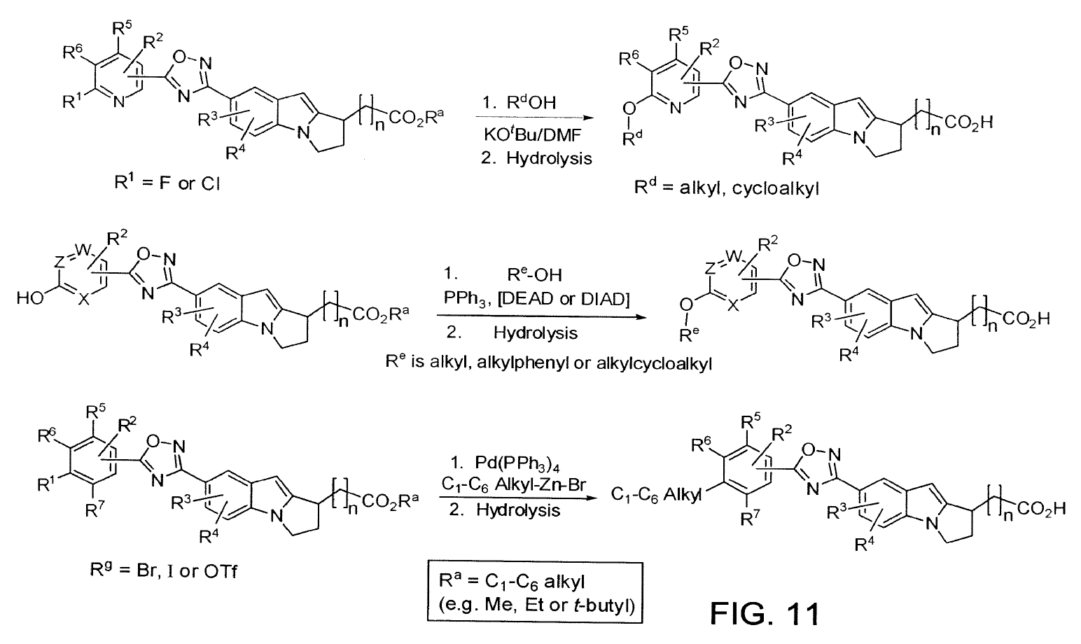

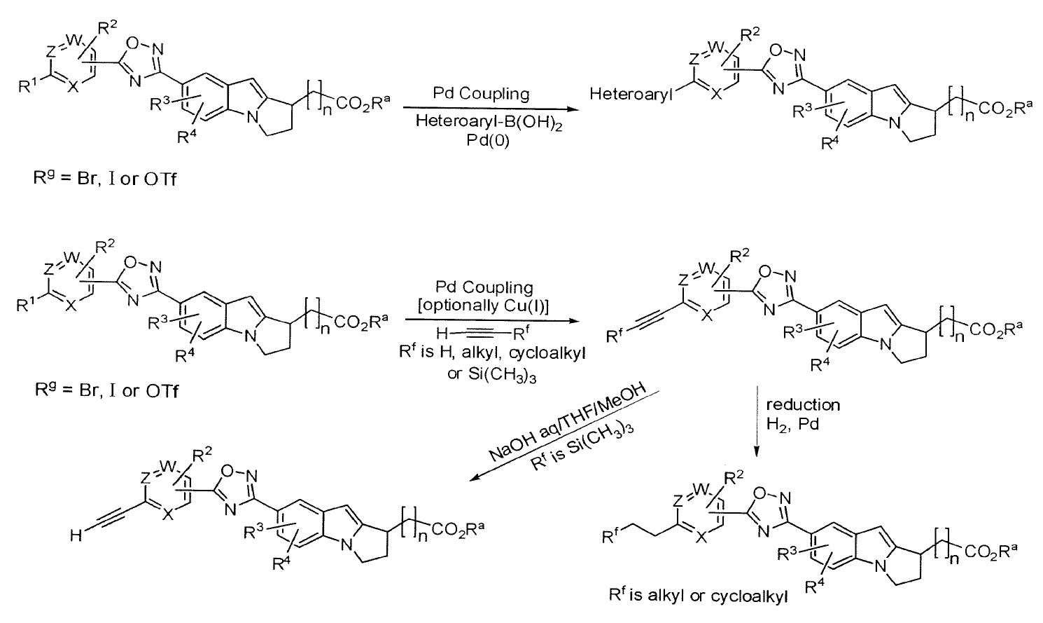

Illustrated syntheses for compounds of the present invention are shown in FIGS. 7 through 12 where the symbols have the same definitions as used throughout this disclosure.

The compounds of the invention and their syntheses are further illustrated by the following examples. The following examples are provided to further define the invention without, however, limiting the invention to the particulars of these examples. The compounds described herein, supra and infra, are named according to the AutoNom version 2.2, or CS ChemDraw Ultra Version 9.0.7. In certain instances common names are used and it is understood that these common names would be recognized by those skilled in the art.

Chemistry: Proton nuclear magnetic resonance (¹H NMR) spectra were recorded on a Bruker Avance-400 equipped with a QNP (Quad Nucleus Probe) or a BBI (Broad Band Inverse) and z-gradient. Chemical shifts are given in parts per million (ppm) with the residual solvent signal used as reference. NMR abbreviations are used as follows: s=singlet, d=doublet, dd=doublet of doublets, ddd=doublet of doublet of doublets, dt=doublet of triplets, t=triplet, td=triplet of doublets, tt=triplet of triplets, q=quartet, m=multiplet, bs=broad singlet, bt=broad triplet. Microwave irradiations were carried out using a Smith Synthesizer™ or an Emrys Optimizer™ (Biotage). Thin-layer chromatography (TLC) was performed on silica gel 60 F₂₅₄ (Merck), preparatory thin-layer chromatography (prep TLC) was preformed on PK6F silica gel 60 A 1 mm plates (Whatman) and column chromatography was carried out on a silica gel column using Kieselgel 60, 0.063-0.200 mm (Merck). Evaporation was done under reduced pressure on a Büchi rotary evaporator.

LCMS spec: HPLC-pumps: LC-10AD VP, Shimadzu Inc.; HPLC system controller: SCL-10A VP, Shimadzu Inc; UV-Detector: SPD-10A VP, Shimadzu Inc; Autosampler: CTC HTS, PAL, Leap Scientific; Mass spectrometer: API 150EX with Turbo Ion Spray source, AB/MDS Sciex; Software: Analyst 1.2.

### Example 1.1

### Preparation of 2-(7-(5-(6-Cyanopyridin-3-yl)-1,2,4-oxadiazol-3-yl)-2,3-dihydro-1H-pyrrolo[1,2-a]indol-1-yl)acetic Acid (Compound 3)

### Step A: Preparation of tert-Butyl 2-(7-(5-(6-Cyanopyridin-3-yl)-1,2,4-oxadiazol-3-yl)-2,3-dihydro-1H-pyrrolo[1,2-a]indol-1-yl)acetate

To a solution of tert-butyl 2-(7-(N′-hydroxycarbamimidoyl)-2,3-dihydro-1H-pyrrolo[1,2-a]indol-1-yl)acetate (50 mg, 0.152 mmol) in dioxane (914 μl) and 6-cyanonicotinic acid (45.0 mg, 0.304 mmol) was added TEA (212 μL, 1.518 mmol) followed by addition of 1-propylphosphonic acid cyclic anhydride (49.2 μL, 0.167 mmol). The reaction mixture was stirred for 30 min, heated to reflux for 18 h, and then diluted with water. The resultant precipitate was filtered off and washed with water to provide the title compound as a yellow solid (43 mg). LCMS m/z=442.4 [M+H]⁺.

### Step B: Preparation of 2-(7-(5-(6-Cyanopyridin-3-yl)-1,2,4-oxadiazol-3-yl)-2,3-dihydro-1H-pyrrolo[1,2-a]indol-1-yl)acetic Acid (Compound 3)

To a solution of tert-butyl 2-(7-(5-(6-cyanopyridin-3-yl)-1,2,4-oxadiazol-3-yl)-2,3-dihydro-1H-pyrrolo[1,2-a]indol-1-yl)acetate (43.1 mg, 0.098 mmol) in DCM (325 μL) was added thioanisole (107 μL, 0.976 mmol) and TFA (150 μL, 1.953 mmol). The reaction mixture was stirred for 1 h. The solvent was removed under reduced pressure. The residue was purified via preparative HPLC/MS. Appropriate fractions were pooled and acetonitrile was removed under reduced pressure. The solution was extracted with EtOAc (3×). The combined EtOAc extracts were washed with brine, dried over MgSO₄ and concentrated to provide the title compound as a light yellow solid (7.3 mg). LCMS m/z=385.9 [M+H]⁺; ¹H NMR (400 MHz, DMSO-d₆) δ ppm 2.24-2.31 (m, 1H), 2.58-2.90 (m, 3H), 3.60-3.73 (m, 1H), 4.06-4.12 (m, 1H), 4.21-4.27 (m, 1H), 6.32 (s, 1H), 7.52 (d, J=8.59 Hz, 1H), 7.82 (dd, J=8.46, 1.64 Hz, 1H), 8.30 (d, J=1.52 Hz, 1H), 8.34 (dd, J=8.08, 0.76 Hz, 1H), 8.80 (dd, J=8.08, 2.02 Hz, 1H), 9.49 (d, J=1.52 Hz, 1H), 12.37 (s, 1H).

### Example 1.2

### Preparation of 2-(7-(5-(3-Cyano-5-methoxyphenyl)-1,2,4-oxadiazol-3-yl)-2,3-dihydro-1H-pyrrolo[1,2-a]indol-1-yl)acetic Acid (Compound 4)

### Step A: Preparation of 3-Cyano-5-hydroxybenzoic Acid

To a solution of 3-bromo-5-hydroxybenzoic acid (1.00 g, 4.61 mmol) in NMP (11.52 mL) was added CuCN (0.413 g, 4.61 mmol). The reaction mixture was heated under microwave irradiation at 200° C. for 2 h. The resultant mixture was purified via preparative HPLC/MS. Appropriate fractions were pooled and acetonitrile was removed under reduced pressure. The solution was extracted with EtOAc (3×). The combined EtOAc extracts were washed with brine, dried over MgSO₄ and concentrated to provide the title compound as a light yellow solid (0.566 g). LCMS m/z=164.1. [M+H]⁺; ¹H NMR (400 MHz, DMSO-d₆) δ ppm 7.38 (dd, J=2.53, 1.52 Hz, 1H), 7.61 (dd, J=2.40, 1.39 Hz, 1H), 7.70 (t, J=1.39 Hz, 1H), 10.61 (s, 1H), 13.43 (s, 1H).

### Step B: Preparation of Methyl 3-Cyano-5-methoxybenzoate

To a solution of 3-cyano-5-hydroxybenzoic acid (0.245 g, 1.5 mmol) in acetonitrile (3 mL) was added Cs₂CO₃ (0.977 g, 3.00 mmol), followed by MeI (0.206 ml, 3.30 mmol). The reaction mixture was stirred for 18 h. The mixture was concentrated under reduced pressure and water and EtOAc were added. The EtOAc extracts were washed with NaHCO₃ (2×) and brine, dried over MgSO₄ and concentrated to provide the title compound as a white solid (0.250 g). ¹H NMR (400 MHz, CDCl₃) δ ppm 3.89 (s, 3H), 3.95 (s, 3H), 7.33 (dd, J=2.65, 1.39 Hz, 1H), 7.78 (dd, J=2.65, 1.39 Hz, 1H), 7.91 (t, J=1.39 Hz, 1H).

### Step C: Preparation of 3-Cyano-5-methoxybenzoic Acid

To a solution of methyl 3-cyano-5-methoxybenzoate (200 mg, 1.046 mmol) in dioxane (2325 μL)/water (1162 μL) was added 1M NaOH (1151 μL, 1.151 mmol). The reaction mixture was stirred for 1 h, quenched with HCl, and extracted with EtOAc (3×). The combined extracts were washed with brine, dried over MgSO₄, and concentrated to provide the title compound as a white solid (0.185 g). ¹H NMR (400 MHz, DMSO-d₆) δ ppm 3.88 (s, 3H), 7.68-7.74 (m, 2H), 7.85 (t, J=1.39 Hz, 1H), 13.58 (s, 1H).

### Step D: Preparation of tert-Butyl 2-(7-(5-(3-Cyano-5-methoxyphenyl)-1,2,4-oxadiazol-3-yl)-2,3-dihydro-1H-pyrrolo[1,2-a]indol-1-yl)acetate

From tert-butyl 2-(7-(N′-hydroxycarbamimidoyl)-2,3-dihydro-1H-pyrrolo[1,2-a]indol-1-yl)acetate and 3-cyano-5-methoxybenzoic acid, using a similar method to the one described in Example 1.1, Step A, the title compound was obtained. LCMS m/z=471.5 [M+H]⁺.

### Step E: Preparation of 2-(7-(5-(3-Cyano-5-methoxyphenyl)-1,2,4-oxadiazol-3-yl)-2,3-dihydro-1H-pyrrolo[1,2-a]indol-1-yl)acetic Acid (Compound 4)

To a solution of tert-butyl 2-(7-(5-(3-cyano-5-methoxyphenyl)-1,2,4-oxadiazol-3-yl)-2,3-dihydro-1H-pyrrolo[1,2-a]indol-1-yl)acetate (97 mg, 0.206 mmol) in CH₂Cl₂ (687 μL) was added triisopropylsilane (424 μL, 2.062 mmol) and TFA (794 μL, 10.31 mmol). The reaction mixture was stirred overnight and purified by preparative HPLC/MS. Appropriate fractions were pooled and acetonitrile was removed under reduced pressure. The remaining aqueous solution was extracted with EtOAc (3×). The EtOAc extracts were washed with brine, dried over MgSO₄ and concentrated to give a pink solid. LCMS m/z=415.3 [M+H]⁺; ¹H NMR (400 MHz, DMSO-d₆) δ ppm 2.21-2.31 (m, 1H), 2.63 (dd, J=16.42, 7.83 Hz, 1H), 2.72-2.90 (m, 2H), 3.61-3.70 (m, 1H), 3.97 (s, 3H), 4.04-4.13 (m, 111), 4.19-4.28 (m, 1H), 6.31 (s, 1H), 7.50 (d, J=8.59 Hz, 1H), 7.81 (dd, J=8.46, 1.64 Hz, 1H), 7.84 (dd, J=2.53, 1.26 Hz, 1H), 7.97 (dd, J=2.53, 1.52 Hz, 1H), 8.18 (t, J=1.39 Hz, 1H), 8.29 (d, J=1.01 Hz, 1H), 12.36 (s, 1H).

### Example 1.3

### Preparation of 2-(7-(5-(3-Cyano-4-isopropoxyphenyl)-1,2,4-oxadiazol-3-yl)-2,3-dihydro-1H-pyrrolo[1,2-a]indol-1-yl)acetic Acid (Compound 5)

### Step A: Preparation of tert-Butyl 2-(7-(5-(3-Cyano-5-isopropoxyphenyl)-1,2,4-oxadiazol-3-yl)-2,3-dihydro-1H-pyrrolo[1,2-a]indol-1-yl)acetate

From tert-butyl 2-(7-(N′-hydroxycarbamimidoyl)-2,3-dihydro-1H-pyrrolo[1,2-a]indol-1-yl)acetate and 3-cyano-4-isopropoxybenzoic acid, using a similar method to the one described in Example 1.1, Step A, the title compound was obtained as a white solid. LCMS m/z=499.8 [M+H]⁺.

### Step B: Preparation of 2-(7-(5-(3-Cyano-4-isopropoxyphenyl)-1,2,4-oxadiazol-3-yl)-2,3-dihydro-1H-pyrrolo[1,2-a]indol-1-yl)acetic Acid (Compound 5)

To a solution of tert-butyl 2-(7-(5-(3-cyano-4-isopropoxyphenyl)-1,2,4-oxadiazol-3-yl)-2,3-dihydro-1H-pyrrolo[1,2-a]indol-1-yl)acetate (94 mg, 0.189 mmol) in CH₂Cl₂ (628 μL) was added triisopropylsilane (388 μL, 1.885 mmol) and TFA (726 μL, 9.43 mmol). The reaction mixture was stirred for 1 h and purified by preparative HPLC/MS. Appropriate fractions were pooled and acetonitrile was removed under reduced pressure. The remaining aqueous solution was extracted with EtOAc (3×), The EtOAc extracts were washed with brine, dried over MgSO₄ and concentrated to give a pink solid. LCMS m/z=443.5 [M+H]⁺; ¹H NMR (400 MHz, DMSO-d₆) δ ppm 1.39 (d, J=6.06 Hz, 6H), 2.22-2.31 (m, 1H), 2.58-2.66 (m, 1H), 2.72-2.80 (m, 1H), 2.78-2.87 (m, 1H), 3.60-3.71 (m, 1H), 4.04-4.12 (m, 1H), 4.20-4.27 (m, 1H), 4.94-5.02 (m, 1H), 6.30 (s, 1H), 7.49 (d, J=8.34 Hz, 1H), 7.56 (d, J=9.09 Hz, 1H), 7.79 (dd, J=8.46, 1.64 Hz, 1H), 8.27 (d, J=1.26 Hz, 1H), 8.41 (dd, J=9.09, 2.27 Hz, 1H), 8.50 (d, J=2.27 Hz, 1H), 12.36 (s, 1H).

Resolution via Chiral HPLC.
Column: normal phase preparative ChiralPak AD-H, 250×20 mm ID, 5 μm particle size
Eluent: 100% acetonitrile with 0.05% trifluoroacetic acid
Gradient: isocratic
Flow: 30 mL/min
Detector: 254 nM
Retention Times: 1^{st} enantiomer: 9.7 min.; 2^{nd} enantiomer: 14.5 min.

### Example 1.4

### Preparation of 2-(7-(5-(Benzo[d][1,3]dioxol-5-yl)-1,2,4-oxadiazol-3-yl)-2,3-dihydro-1H-pyrrolo[1,2-a]indol-1-yl)acetic Acid (Compound 6)

### Step A: Preparation of tert-Butyl 2-(7-(5-(Benzo[d][1,3]dioxol-5-yl)-1,2,4-oxadiazol-3-yl)-2,3-dihydro-1H-pyrrolo[1,2-a]indol-1-yl)acetate

To a solution of tert-butyl 2-(7-(N′-hydroxycarbamimidoyl)-2,3-dihydro-1H-pyrrolo[1,2-a]indol-1-yl)acetate (50 mg, 0.152 mmol) in dioxane (914 μL) and TEA (74.0 μL, 0.531 mmol) was added benzo[d][1,3]dioxole-5-carbonyl chloride (30.8 mg, 0.167 mmol). The reaction mixture was heated to reflux for 18 h, and then diluted with water. The resultant precipitate was filtered off and washed with water to provide the title compound as a brown solid (52.4 mg). LCMS m/z=460.2 [M+H]⁺.

### Step B: Preparation of 2-(7-(5-(Benzo[d][1,3]dioxol-5-yl)-1,2,4-oxadiazol-3-yl)-2,3-dihydro-1H-pyrrolo[1,2-a]indol-1-yl)acetic Acid (Compound 6)

From tert-butyl 2-(7-(5-(benzo[d][1,3]dioxol-5-yl)-1,2,4-oxadiazol-3-yl)-2,3-dihydro-1H-pyrrolo[1,2-a]indol-1-yl)acetate, using a similar method to the one described in Example 1.3, Step B, the title compound was obtained as a white solid. LCMS m/z=404.2 [M+H]⁺; ¹H NMR (400 MHz, DMSO-d₆) δ ppm 2.21-2.31 (m, 1H), 2.58-2.66 (m, 1H), 2.72-2.88 (m, 2H), 3.60-3.72 (m, 1H), 4.04-4.12 (m, 1H), 4.19-4.26 (m, 1H), 6.21 (s, 2H), 6.30 (s, 1H), 7.19 (d, J=8.08 Hz, 1H), 7.48 (d, J=8.34 Hz, 1H), 7.65 (d, J=1.77 Hz, 1H), 7.76-7.78 (m, 1H), 7.79 (t, J=1.89 Hz, 1H), 8.25 (d, J=1.01 Hz, 1H), 12.36 (s, 1H).

### Example 1.5

### Preparation of 2-(7-(5-(2,6-Dimethoxypyridin-3-yl)-1,2,4-oxadiazol-3-yl)-2,3-dihydro-1H-pyrrolo[1,2-a]indol-1-yl)acetic Acid (Compound 7)

From tert-butyl 2-(7-(N′-hydroxycarbamimidoyl)-2,3-dihydro-1H-pyrrolo[1,2-a]indol-1-yl)acetate and 2,6-dimethoxynicotinic acid, using a similar method (HPLC fractions were lyophilized) to the one described in Example 1.2, Steps D and E, the title compound was obtained as a white solid. LCMS m/z=421.3 [M+H]⁺; ¹H NMR (400 MHz, DMSO-d₆) δ ppm 2.21-2.31 (m, 1H), 2.58-2.66 (m, 1H), 2.71-2.87 (m, 2H), 3.60-3.70 (m, 1H), 4.00 (s, 3H), 4.03-4.12 (m, 1H), 4.09 (s, 3H), 4.19-4.27 (m, 1H), 6.29 (s, 1H), 6.65 (d, J=8.59 Hz, 1H), 7.47 (d, J=8.34 Hz, 1H), 7.77 (dd, J=8.34, 1.52 Hz, 1H), 8.24 (d, J=1.26 Hz, 1H), 8.44 (d, J=8.34 Hz, 1H), 12.37 (s, 1H).

### Example 1.6

### Preparation of 2-(7-(5-(2,2-Difluorobenzo[d][1,3]dioxol-5-yl)-1,2,4-oxadiazol-3-yl)-2,3-dihydro-1H-pyrrolo[1,2-a]indol-1-yl)acetic Acid (Compound 8)

From tert-butyl 2-(7-(N′-hydroxycarbamimidoyl)-2,3-dihydro-1H-pyrrolo[1,2-a]indol-1-yl)acetate and 2,2-difluorobenzo[d][1,3]dioxole-5-carbonyl chloride, using a similar method to the one described in Example 1.4, the title compound was obtained as a white solid. LCMS m/z=440.2 [M+H]⁺; ¹H NMR (400 MHz, DMSO-d₆) δ ppm 2.22-2.31 (m, 1H), 2.63 (dd, J=16.42, 7.83 Hz, 1H), 2.72-2.90 (m, 2H), 3.60-3.70 (m, 1H), 4.01-4.12 (m, 1H), 4.19-4.28 (m, 1H), 6.31 (s, 1H), 7.50 (d, J=8.59 Hz, 1H), 7.72 (d, J=8.34 Hz, 1H), 7.80 (dd, J=8.46, 1.64 Hz, 1H), 8.12 (dd, J=8.46, 1.64 Hz, 1H), 8.23 (d, J=1.77 Hz, 1H), 8.27 (d, J=1.26 Hz, 1H), 12.36 (s, 1H).

### Example 1.7

### Preparation of 2-(7-(5-(4-Methylpyridin-3-yl)-1,2,4-oxadiazol-3-yl)-2,3-dihydro-1H-pyrrolo[1,2-a]indol-1-yl)acetic Acid (Compound 10)

From tert-butyl 2-(7-(N′-hydroxycarbamimidoyl)-2,3-dihydro-1H-pyrrolo[1,2-a]indol-1-yl)acetate and 4-methylnicotinic acid, using a similar method to the one described in Example 1.5, the title compound was obtained as a pink solid. LCMS m/z=375.1 [M+H]⁺; ¹H NMR (400 MHz, DMSO-d₆) δ ppm 2.23-2.31 (m, 1H), 2.59-2.67 (m, 1H), 2.70-2.89 (m, 2H), 2.77 (s, 3H), 3.60-3.71 (m, 1H), 4.04-4.14 (m, 1H), 4.20-4.27 (m, 1H), 6.32 (s, 1H), 7.50 (d, J=8.34 Hz, 1H), 7.58 (d, J=5.31 Hz, 1H), 7.82 (dd, J=8.46, 1.64 Hz, 1H), 8.30 (s, 1H), 8.71 (d, J=5.05 Hz, 1H), 9.23 (s, 1H).

### Example 1.8

### Preparation of 2-(7-(5-(4-(Trifluoromethyl)pyridin-3-yl)-1,2,4-oxadiazol-3-yl)-2,3-dihydro-1H-pyrrolo[1,2-a]indol-1-yl)acetic Acid (Compound 11)

From tert-butyl 2-(7-(N′-hydroxycarbamimidoyl)-2,3-dihydro-1H-pyrrolo[1,2-a]indol-1-yl)acetate and 4-trifluoromethylnicotinic acid, using a similar method to the one described in Example 1.5, the title compound was obtained as a yellow solid. LCMS m/z=429.1 [M+H]⁺; ¹H NMR (400 MHz, DMSO-d₆) δ ppm 2.22-2.31 (m, 1H), 2.58-2.69 (m, 1H), 2.70-2.90 (m, 2H), 3.60-3.73 (m, 1H), 4.02-4.14 (m, 1H), 4.18-4.31 (m, 1H), 6.32 (s, 1H), 7.52 (d, J=8.59 Hz, 1H), 7.81 (dd, J=8.34, 1.52 Hz, 1H), 8.12 (d, J=5.31 Hz, 1H), 8.28 (d, J=1.01 Hz, 1H), 9.18 (d, J=5.05 Hz, 1H), 9.44 (s, 1H), 12.35 (s, 1H).

### Example 1.9

### Preparation of 2-(7-(5-(6-Methylpyridin-3-yl)-1,2,4-oxadiazol-3-yl)-2,3-dihydro-1H-pyrrolo[1,2-a]indol-1-yl)acetic Acid (Compound 12)

From tert-butyl 2-(7-(N′-hydroxycarbamimidoyl)-2,3-dihydro-1H-pyrrolo[1,2-a]indol-1-yl)acetate and 6-methylnicotinic acid, using a similar method to the one described in Example 1.5, the title compound was obtained as a light yellow solid. LCMS m/z=375.3 [M+H]⁺; ¹H NMR (400 MHz, DMSO-d₆) δ ppm 2.22-2.31 (m, 1H), 2.57-2.69 (m, 1H), 2.62 (s, 3H), 2.72-2.88 (m, 2H), 3.60-3.70 (m, 1H), 4.04-4.13 (m, 1H), 4.19-4.28 (m, 1H), 6.31 (s, 1H), 7.50 (d, J=8.59 Hz, 1H), 7.58 (d, J=8.34 Hz, 1H), 7.81 (dd, J=8.34, 1.52 Hz, 1H), 8.28 (d, J=1.01 Hz, 1H), 8.45 (dd, J=8.08, 2.27 Hz, 1H), 9.23 (d, J=2.02 Hz, 1H).

### Example 1.10

### Preparation of 2-(7-(5-(3-Hydroxypyridin-2-yl)-1,2,4-oxadiazol-3-yl)-2,3-dihydro-1H-pyrrolo[1,2-a]indol-1-yl)acetic Acid (Compound 13)

From tert-butyl 2-(7-(N′-hydroxycarbamimidoyl)-2,3-dihydro-1H-pyrrolo[1,2-a]indol-1-yl)acetate and 3-hydroxypicolinic acid, using a similar method to the one described in Example 1.5, the title compound was obtained as a white solid. LCMS m/z=377.2 [M+H]⁺; ¹H NMR (400 MHz, DMSO-d₆) δ ppm 2.21-2.31 (m, 1H), 2.59-2.67 (m, 1H), 2.72-2.88 (m, 2H), 3.61-3.70 (m, 1H), 4.05-4.13 (m, 1H), 4.20-4.28 (m, 1H), 6.31 (s, 1H), 7.50 (d, J=8.59 Hz, 1H), 7.60-7.62 (m, 2H), 7.83 (dd, J=8.34, 1.52 Hz, 1H), 8.32 (d, J=1.26 Hz, 1H), 8.33-8.36 (m, 1H), 10.81 (s, 1H), 12.36 (s, 1H).

### Example 1.11

### Preparation of 2-(7-(5-(5-Methylpyridin-3-yl)-1,2,4-oxadiazol-3-yl)-2,3-dihydro-1H-pyrrolo[1,2-a]indol-1-yl)acetic Acid (Compound 14)

From tert-butyl 2-(7-(N′-hydroxycarbamimidoyl)-2,3-dihydro-1H-pyrrolo[1,2-a]indol-1-yl)acetate and 5-methylnicotinic acid, using a similar method to the one described in Example 1.5, the title compound was obtained as a yellow solid. LCMS m/z=375.3 [M+H]⁺; ¹H NMR (400 MHz, DMSO-d₆) δ ppm 2.21-2.31 (m, 1H), 2.46 (s, 3H), 2.56-2.70 (m, 1H), 2.69-2.88 (m, 2H), 3.60-3.70 (m, 1H), 4.04-4.14 (m, 1H), 4.18-4.30 (m, 1H), 6.32 (s, 1H), 7.50 (d, J=8.59 Hz, 1H), 7.81 (dd, J=8.34, 1.52 Hz, 1H), 8.29 (d, J=1.01 Hz, 1H), 8.41 (s, 1H), 8.74 (d, J=1.52 Hz, 1H), 9.16 (d, J=2.02 Hz, 1H), 12.38 (bs, 1H).

### Example 1.12

### Preparation of 2-(7-(5-(3-Cyano-4-(2,2,2-trifluoroethoxy)phenyl)-1,2,4-oxadiazol-3-yl)-2,3-dihydro-1H-pyrrolo[1,2-a]indol-1-yl)acetic Acid (Compound 15)

### Step A: Preparation of Sodium 2,2,2-Trifluoroethanolate

To a cooled (0° C.) solution of 2,2,2-trifluoroethanol (1.439 mL, 20 mmol) in THF (36.4 ml) was added NaH (0.727 g, 18.18 mmol). The reaction mixture was stirred for 1 h while warming to room temperature. The mixture was concentrated under reduced pressure, triturated in hexanes, and filtered to provide the title compound as a white solid (2.21 g).

### Step B: Preparation of Methyl 3-Cyano-4-(2,2,2-trifluoroethoxy)benzoate

To a solution of methyl 3-cyano-4-fluorobenzoate (0.327 g, 1.825 mmol) in dioxane (3.65 mL) was added sodium 2,2,2-trifluoroethanolate (0.234 g, 1.917 mmol). The reaction mixture was heated to 100° C. under microwave irradiation for 2 h. The mixture was diluted with water and extracted with EtOAc (3×). The combined extracts were washed with brine, dried over MgSO₄, and concentrated. The residue was purified via column chromatography (1:3 EtOAc/hexanes) to provide the title compound as a white solid (0.188 g). ¹H NMR (400 MHz, CDCl₃) δ ppm 3.94 (s, 3H), 4.56 (q, J=7.58 Hz, 2H), 7.04 (d, J=8.84 Hz, 1H), 8.26 (dd, J=8.84, 2.27 Hz, 1H), 8.32 (d, J=2.27 Hz, 1H).

### Step C: Preparation of 3-Cyano-4-(2,2,2-trifluoroethoxy)benzoic Acid

From methyl 3-cyano-4-(2,2,2-trifluoroethoxy)benzoate, using a similar method to the one described in Example 1.2, Step C, the title compound was obtained as a white solid. ¹H NMR (400 MHz, DMSO-d₆) δ ppm 5.10 (q, J=8.67 Hz, 2H), 7.49 (d, J=8.84 Hz, 1H), 8.21-8.25 (m, 1H), 8.27 (d, J=2.02 Hz, 1H), 13.34 (s, 1H).

### Step D: Preparation of 2-(7-(5-(3-Cyano-4-(2,2,2-trifluoroethoxy)phenyl)-1,2,4-oxadiazol-3-yl)-2,3-dihydro-1H-pyrrolo[1,2-a]indol-1-yl)acetic Acid (Compound 15)

From tert-butyl 2-(7-(N′-hydroxycarbamimidoyl)-2,3-dihydro-1H-pyrrolo[1,2-a]indol-1-yl)acetate and 3-cyano-4-(2,2,2-trifluoroethoxy)benzoic acid, using a similar method to the one described in Example 1.5, the title compound was obtained as a yellow solid. LCMS m/z=483.4 [M+H]⁺; ¹H NMR (400 MHz, DMSO-d₆) δ ppm 2.21-2.31 (m, 1H), 2.63 (dd, J=16.42, 8.08 Hz, 1H), 2.72-2.88 (m, 2H), 3.60-3.71 (m, 1H), 4.02-4.13 (m, 1H), 4.18-4.29 (m, 1H), 5.17 (q, J=8.76 Hz, 2H), 6.31 (s, 1H), 7.50 (d, J=8.59 Hz, 1H), 7.66 (d, J=9.09 Hz, 1H), 7.80 (dd, J=8.46, 1.64 Hz, 1H), 8.28 (d, J=1.26 Hz, 1H), 8.52 (dd, J=8.84, 2.27 Hz, 1H), 8.61 (d, J=2.27 Hz, 1H), 12.36 (s, 1H).

### Example 1.13

### Preparation of 2-(7-(5-(4-Methylpyridin-2-yl)-1,2,4-oxadiazol-3-yl)-2,3-dihydro-1H-pyrrolo[1,2-a]indol-1-yl)acetic Acid (Compound 16)

From tert-butyl 2-(7-(N′-hydroxycarbamimidoyl)-2,3-dihydro-1H-pyrrolo[1,2-a]indol-1-yl)acetate and 4-methylpicolinic acid, using a similar method to the one described in Example 1.5, the title compound was obtained as a yellow solid. LCMS m/z=375.2 [M+H]⁺; ¹H NMR (400 MHz, DMSO-d₆) δ ppm 2.22-2.31 (m, 1H), 2.59-2.66 (m, 1H), 2.73-2.87 (m, 2H), 3.17 (s, 3H), 3.61-3.71 (m, 1H), 4.02-4.14 (m, 111), 4.18-4.32 (m, 1H), 6.31 (s, 1H), 7.51 (d, J=8.59 Hz, 1H), 7.57 (d, J=4.80 Hz, 1H), 7.82 (dd, J=8.34, 1.52 Hz, 1H), 8.21-8.24 (m, 1H), 8.29 (d, J=1.01 Hz, 1H), 8.70 (d, J=5.05 Hz, 1H), 12.36 (bs, 1H).

### Example 1.14

### Preparation of 2-(7-(5-(6-(1H-Pyrazol-1-yl)pyridin-3-yl)-1,2,4-oxadiazol-3-yl)-2,3-dihydro-1H-pyrrolo[1,2-a]indol-1-yl)acetic Acid (Compound 17)

From tert-butyl 2-(7-(N′-hydroxycarbamimidoyl)-2,3-dihydro-1H-pyrrolo[1,2-a]indol-1-yl)acetate and 6-(1H-pyrazol-1-yl)nicotinic acid, using a similar method to the one described in Example 1.5, the title compound was obtained as a white solid. LCMS m/z=427.4 [M+H]⁺; ¹H NMR (400 MHz, DMSO-d₆) δ ppm 2.21-2.31 (m, 1H), 2.58-2.67 (m, 1H), 2.72-2.89 (m, 2H), 3.60-3.72 (m, 1H), 4.04-4.14 (m, 1H), 4.19-4.29 (m, 1H), 6.32 (s, 1H), 6.69 (dd, J=2.65, 1.64 Hz, 1H), 7.51 (d, J=8.34 Hz, 1H), 7.82 (dd, J=8.46, 1.64 Hz, 1H), 7.96-7.97 (m, 1H), 8.18 (d, J=8.84 Hz, 1H), 8.29 (d, J=1.26 Hz, 1H), 8.72 (dd, J=8.84, 2.27 Hz, 1H), 8.75 (d, J=2.78 Hz, 1H), 9.24 (d, J=2.27 Hz, 1H), 12.36 (s, 1H).

### Example 1.15

### Preparation of 2-(7-(5-(3-Bromo-5-(trifluoromethoxy)phenyl)-1,2,4-oxadiazol-3-yl)-2,3-dihydro-1H-pyrrolo[1,2-a]indol-1-yl)acetic Acid (Compound 18)

### Step A: Preparation of 3-Bromo-5-(trifluoromethoxy)benzoyl Chloride

To a cooled (0° C.) mixture of 3-bromo-5-(trifluoromethoxy)benzoic acid (1.425 g, 5.00 mmol) in DCM (10.00 mL) was added oxalyl chloride (0.481 mL, 5.50 mmol) followed by one drop of DMF. The reaction mixture was stirred for 2 h while warming to room temperature and concentrated under reduced pressure to give the title compound without further purification.

### Step B: Preparation of 2-(7-(5-(3-Bromo-5-(trifluoromethoxy)phenyl)-1,2,4-oxadiazol-3-yl)-2,3-dihydro-1H-pyrrolo[1,2-a]indol-1-yl)acetic Acid (Compound 18)

From tert-butyl 2-(7-(N′-hydroxycarbamimidoyl)-2,3-dihydro-1H-pyrrolo[1,2-a]indol-1-yl)acetate and 3-bromo-5-(trifluoromethoxy)benzoyl chloride, using a similar method to the one described in Example 1.4, the title compound was obtained as a white solid. LCMS m/z=522.3 [M+H]⁺; ¹H NMR (400 MHz, DMSO-d₆) δ ppm 2.22-2.31 (m, 1H), 2.59-2.66 (m, 1H), 2.72-2.89 (m, 2H), 3.60-3.70 (m, 1H), 4.02-4.13 (m, 1H), 4.19-4.28 (m, 1H), 6.31 (s, 1H), 7.50 (d, J=8.59 Hz, 1H), 7.81 (dd, J=8.46, 1.64 Hz, 1H), 8.10-8.15 (m, 2H), 8.29 (d, J=1.52 Hz, 1H), 8.38 (t, J=1.52 Hz, 1H), 12.36 (s, 1H).

### Example 1.16

### Preparation of 2-(7-(5-(2-Methylpyridin-3-yl)-1,2,4-oxadiazol-3-yl)-2,3-dihydro-1H-pyrrolo[1,2-a]indol-1-yl)acetic Acid (Compound 21)

From tert-butyl 2-(7-(N′-hydroxycarbamimidoyl)-2,3-dihydro-1H-pyrrolo[1,2-a]indol-1-yl)acetate and 2-methylnicotinic acid, using a similar method to the one described in Example 1.5, the title compound was obtained as a white solid. LCMS m/z=375.0 [M+H]⁺; ¹H NMR (400 MHz, DMSO-d₆) δ ppm 2.18-2.34 (m, 1H), 2.55-2.66 (m, 1H), 2.70-2.90 (m, 2H), 2.96 (s, 3H), 3.60-3.72 (m, 1H), 4.00-4.14 (m, 1H), 4.18-4.29 (m, 1H), 6.32 (s, 1H), 7.50 (d, J=8.59 Hz, 1H), 7.56 (dd, J=8.08, 4.80 Hz, 1H), 7.82 (dd, J=8.46, 1.64 Hz, 1H), 8.29 (d, J=1.26 Hz, 1H), 8.52 (dd, J=7.96, 1.64 Hz, 1H), 8.76 (dd, J=4.93, 1.64 Hz, 1H), 12.29 (s, 1H).

### Example 1.17

### Preparation of 2-(7-(5-(5-Methylpyrazin-2-yl)-1,2,4-oxadiazol-3-yl)-2,3-dihydro-1H-pyrrolo[1,2-a]indol-1-yl)acetic Acid (Compound 22)

From tert-butyl 2-(7-(N′-hydroxycarbamimidoyl)-2,3-dihydro-1H-pyrrolo[1,2-a]indol-1-yl)acetate and 5-methylpyrazine-2-carboxylic acid, using a similar method to the one described in Example 1.5, the title compound was obtained as a yellow solid. LCMS m/z=376.1 [M+H]⁺; ¹H NMR (400 MHz, DMSO-d₆) δ ppm 2.20-2.31 (m, 1H), 2.58-2.68 (m, 1H), 2.67 (s, 3H), 2.72-2.90 (m, 2H), 3.60-3.70 (m, 1H), 4.00-4.14 (m, 1H), 4.18-4.29 (m, 1H), 6.32 (s, 1H), 7.51 (d, J=8.59 Hz, 1H), 7.83 (dd, J=8.46, 1.64 Hz, 1H), 8.30 (d, J=1.26 Hz, 1H), 8.84 (s, 1H), 9.38 (d, J=1.52 Hz, 1H), 12.36 (s, 1H).

### Example 1.18

### Preparation of 2-(7-(5-(6-Methylpyridin-2-yl)-1,2,4-oxadiazol-3-yl)-2,3-dihydro-1H-pyrrolo[1,2-a]indol-1-yl)acetic Acid (Compound 23)

From tert-butyl 2-(7-(N′-hydroxycarbamimidoyl)-2,3-dihydro-1H-pyrrolo[1,2-a]indol-1-yl)acetate and 5-methylpicolinic acid, using a similar method to the one described in Example 1.5, the title compound was obtained as a white solid. LCMS m/z=375.3 [M+H]⁺; ¹H NMR (400 MHz, DMSO-d₆) δ ppm 2.21-2.31 (m, 1H), 2.56-2.68 (m, 1H), 2.63 (s, 3H), 2.72-2.90 (m, 2H), 3.57-3.75 (m, 1H), 4.02-4.13 (m, 1H), 4.18-4.28 (m, 1H), 6.31 (s, 1H), 7.50 (d, J=8.59 Hz, 1H), 7.60 (d, J=7.83 Hz, 1H), 7.82 (dd, J=8.59, 1.52 Hz, 1H), 8.01 (t, J=7.71 Hz, 1H), 8.16 (d, J=7.83 Hz, 1H), 8.29 (s, 1H), 12.36 (s, 1H).

### Example 1.19

### Preparation of 2-(7-(5-(2,2-Difluorobenzo[d][1,3]dioxol-4-yl)-1,2,4-oxadiazol-3-yl)-2,3-dihydro-1H-pyrrolo[1,2-a]indol-1-yl)acetic Acid (Compound 24)

From tert-butyl 2-(7-(N′-hydroxycarbamimidoyl)-2,3-dihydro-1H-pyrrolo[1,2-a]indol-1-yl)acetate and 2,2-difluorobenzo[d][1,3]dioxole-4-carboxylic acid, using a similar method to the one described in Example 1.5, the title compound was obtained as a white solid. LCMS m/z=440.1 [M+H]⁺; ¹H NMR (400 MHz, DMSO-d₆) δ ppm 2.23-2.31 (m, 1H), 2.59-2.66 (m, 1H), 2.73-2.88 (m, 2H), 3.61-3.71 (m, 1H), 4.05-4.13 (m, 1H), 4.20-4.28 (m, 1H), 6.33 (s, 1H), 7.47-7.53 (m, 2H), 7.78 (dd, J=8.08, 1.01 Hz, 1H), 7.81 (dd, J=8.34, 1.52 Hz, 1H), 7.98 (dd, J=8.34, 1.01 Hz, 1H), 8.28 (d, J=1.01 Hz, 1H), 12.36 (s, 1H).

### Example 1.20

### Preparation of 2-(7-(5-(3-(Difluoromethoxy)phenyl)-1,2,4-oxadiazol-3-yl)-2,3-dihydro-1H-pyrrolo[1,2-a]indol-1-yl)acetic Acid (Compound 25)

From tert-butyl 2-(7-(N′-hydroxycarbamimidoyl)-2,3-dihydro-1H-pyrrolo[1,2-a]indol-1-yl)acetate and 3-(difluoromethoxy)benzoic acid, using a similar method to the one described in Example 1.5, the title compound was obtained as a white solid. LCMS m/z=426.2 [M+H]⁺; ¹H NMR (400 MHz, DMSO-d₆) δ ppm 2.20-2.33 (m, 1H), 2.59-2.66 (m, 1H), 2.71-2.89 (m, 2H), 3.60-3.71 (m, 1H), 4.02-4.12 (m, 1H), 4.18-4.29 (m, 1H), 6.32 (s, 1H), 7.50 (d, J=8.34 Hz, 1H), 7.56 (dd, J=8.34, 2.27 Hz, 1H), 7.74 (t, J=8.08 Hz, 1H), 7.81 (dd, J=8.59, 1.52 Hz, 1H), 7.81 (dd, J=8.59, 1.52 Hz, 1H), 7.95 (d, J=1.77 Hz, 1H), 8.08 (d, J=8.08 Hz, 1H), 8.28 (d, J=1.26 Hz, 1H), 12.36 (s, 1H).

### Example 1.21

### Preparation of 2-(7-(5-(6-Acetamidopyridin-3-yl)-1,2,4-oxadiazol-3-yl)-2,3-dihydro-1H-pyrrolo[1,2-a]indol-1-yl)acetic Acid (Compound 26)

From tert-butyl 2-(7-(N′-hydroxycarbamimidoyl)-2,3-dihydro-1H-pyrrolo[1,2-a]indol-1-yl)acetate and 6-acetamidonicotinic acid, using a similar method to the one described in Example 1.5, the title compound was obtained as a brown solid. LCMS m/z=418.4 [M+H]⁺; ¹H NMR (400 MHz, DMSO-d₆) δ ppm 2.17 (s, 3H), 2.22-2.31 (m, 1H), 2.57-2.67 (m, 1H), 2.73-2.88 (m, 2H), 3.60-3.71 (m, 1H), 4.01-4.14 (m, 1H), 4.16-4.29 (m, 1H), 6.31 (s, 1H), 7.49 (d, J=8.34 Hz, 1H), 7.80 (dd, J=8.59, 1.52 Hz, 1H), 8.27 (d, J=1.01 Hz, 1H), 8.33 (d, J=8.84 Hz, 1H), 8.52 (dd, J=8.84, 2.53 Hz, 1H), 9.10 (d, J=1.52 Hz, 1H), 11.02 (s, 1H), 12.32 (bs, 1H).

### Example 1.22

### Preparation of 2-(7-(5-(6-Morpholinopyridin-3-yl)-1,2,4-oxadiazol-3-yl)-2,3-dihydro-1H-pyrrolo[1,2-a]indol-1-yl)acetic Acid (Compound 27)

From tert-butyl 2-(7-(N′-hydroxycarbamimidoyl)-2,3-dihydro-1H-pyrrolo[1,2-a]indol-1-yl)acetate and 6-morpholinonicotinic acid, using a similar method to the one described in Example 1.5, the title compound was obtained as a white solid. LCMS m/z=446.4 [M+H]⁺; ¹H NMR (400 MHz, DMSO-d₆) δ ppm 2.20-2.31 (m, 1H), 2.62 (dd, J=16.29, 7.96 Hz, 1H), 2.71-2.88 (m, 2H), 3.54-3.77 (m, 9H), 4.03-4.12 (m, 1H), 4.19-4.27 (m, 1H), 6.30 (s, 1H), 7.04 (d, J=9.09 Hz, 1H), 7.47 (d, J=8.34 Hz, 1H), 7.78 (dd, J=8.34, 1.52 Hz, 1H), 8.21 (dd, J=9.09, 2.27 Hz, 1H), 8.24 (d, J=1.01 Hz, 1H), 8.89 (d, J=2.27 Hz, 1H), 12.37 (bs, 1H).

### Example 1.23

### Preparation of 2-(7-(5-(5-(Thiophen-2-yl)pyridin-3-yl)-1,2,4-oxadiazol-3-yl)-2,3-dihydro-1H-pyrrolo[1,2-a]indol-1-yl)acetic Acid (Compound 28)

From tert-butyl 2-(7-(N′-hydroxycarbamimidoyl)-2,3-dihydro-1H-pyrrolo[1,2-a]indol-1-yl)acetate and 5-(thiophen-2-yl)nicotinic acid, using a similar method to the one described in Example 1.5, the title compound was obtained as a white solid. LCMS m/z=443.4 [M+H]⁺; ¹H NMR (400 MHz, DMSO-d₆) δ ppm 2.21-2.34 (m, 1H), 2.57-2.67 (m, 1H), 2.72-2.91 (m, 2H), 3.57-3.74 (m, J=7.33 Hz, 1H), 4.03-4.15 (m, 1H), 4.20-4.30 (m, 1H), 6.33 (s, 1H), 7.28 (dd, J=5.05, 3.79 Hz, 1H), 7.51 (d, J=8.59 Hz, 1H), 7.78 (d, J=5.05 Hz, 1H), 7.84 (dd, J=8.59, 1.26 Hz, 1H), 7.91 (d, J=3.54 Hz, 1H), 8.32 (s, 1H), 8.68 (t, J=2.02 Hz, 1H), 9.23 (d, J=2.27 Hz, 1H), 9.25 (d, J=2.02 Hz, 1H), 12.38 (bs, 1H).

### Example 1.24

### Preparation of 2-(7-(5-(3-(Pyridin-3-yl)-5-(trifluoromethoxy)phenyl)-1,2,4-oxadiazol-3-yl)-2,3-dihydro-1H-pyrrolo[1,2-a]indol-1-yl)acetic Acid (Compound 29)

### Step A: Preparation of tert-Butyl 2-(7-(5-(3-(Pyridine-3-yl)-5-(trifluoromethoxy)phenyl)-1,2,4-oxadiazol-3-yl)-2,3-dihydro-1H-pyrrolo[1,2-a]indol-1-yl)acetate

To a solution of tert-butyl 2-(7-(5-(3-bromo-5-(trifluoromethoxy)phenyl)-1,2,4-oxadiazol-3-yl)-2,3-dihydro-1H-pyrrolo[1,2-a]indol-1-yl)acetate (0.058 g, 0.100 mmol) in benzene (0.259 mL)/ethanol (0.074 mL) was added Na₂CO₃ (0.100 mL, 0.200 mmol), pyridin-3-ylboronic acid (0.015 g, 0.120 mmol), and tetrakis(triphenylphosphine)palladium (0) (3.47 mg, 3.00 μmol). The reaction mixture was heated under microwave irradiation at 100° C. for 3 h. It was then diluted with EtOAc, washed with water, aqueous NH₄Cl (3×) and brine, dried over MgSO₄, and concentrated. The residue was purified via column chromatography (1:3 EtOAc/hexanes) to provide the title compound as a white solid (20 mg). LCMS m/z=577.3 [M+H]⁺.

### Step B: Preparation of 2-(7-(5-(3-(pyridin-3-yl)-5-(trifluoromethoxy)phenyl)-1,2,4-oxadiazol-3-yl)-2,3-dihydro-1H-pyrrolo[1,2-a]indol-1-yl)acetic acid (Compound 29)

From tert-butyl 2-(7-(5-(3-(pyridine-3-yl)-5-(trifluoromethoxy)phenyl)-1,2,4-oxadiazol-3-yl)-2,3-dihydro-1H-pyrrolo[1,2-a]indol-1-yl)acetate, using a similar method to the one described in Example 1.5, Step B, the title compound was obtained as a white solid. LCMS m/z=521.6 [M+H]⁺; ¹H NMR (400 MHz, DMSO-d₆) δ ppm 2.23-2.35 (m, 1H), 2.58-2.66 (m, 1H), 2.70-2.93 (m, 2H), 3.61-3.70 (m, 1H), 4.04-4.14 (m, 1H), 4.18-4.29 (m, 1H), 6.32 (s, 1H), 7.51 (d, J=8.34 Hz, 1H), 7.66 (dd, J=7.96, 4.93 Hz, 1H), 7.84 (dd, J=8.34, 1.26 Hz, 1H), 8.19 (s, 2H), 8.32 (s, 1H), 8.41 (d, J=8.34 Hz, 1H), 8.55 (s, 1H), 8.69-8.78 (m, 1H), 9.13 (d, J=2.27 Hz, 1H), 12.35 (s, 1H).

### Example 1.25

### Preparation of 2-(7-(5-(3-Cyano-5-(trifluoromethoxy)phenyl)-1,2,4-oxadiazol-3-yl)-2,3-dihydro-1H-pyrrolo[1,2-a]indol-1-yl)acetic Acid (Compound 2)

### Step A: Preparation of 7-Bromo-2,3-dihydro-1H-pyrrolo[1,2-a]indol-1-one

To a solution of ethyl 5-bromo-1H-indole-2-carboxylate (30 g, 112 mmol) in toluene (500 mL) was added portionwise sodium hydride (60% dispersion in mineral oil, 9.40 g, 235 mmol). Vigorous gas evolution was observed. The resulting white suspension was heated to 110° C. Butyl acrylate (35.1 mL, 246 mmol) was added dropwise (using a syringe pump) over 24 h while stirring vigorously at an internal temperature of 110° C. Additional butyl acrylate (10 mL) was added in one portion and stirring was continued at 110° C. for 4 h followed by additional sodium hydride (60% dispersion in mineral oil, 5 g) and butyl acrylate (10 mL). 4 h later another 6 mL of butyl acrylate was added. Stirring was continued at 110° C. for a total of 48 h. The reaction was cooled in an ice-bath, and 2 M HCl (400 mL) was added carefully. The layers were separated and the aqueous layer was extracted with dichloromethane (2×200 mL). The combined organic extracts were washed with brine, dried over MgSO₄, filtered, and concentrated. The resulting orange residue was dissolved in acetic acid (900 mL) and water (100 mL). The orange solution was refluxed for 16 h. The solvents were removed under reduced pressure. To the residue was added dichloromethane (300 mL). The resulting precipitate was collected by filtration and rinsed twice with dichloromethane to provide the title compound. LCMS m/z=250.2 [M+H]⁺; ¹H NMR (400 MHz, DMSO-d₆) δ ppm 3.20 (t, J=6.1 Hz, 2H), 4.46 (t, J=6.1 Hz, 2H), 6.92 (s, 1H), 7.46 (dd, J=8.8, 1.8 Hz, 1H), 7.63 (d, J=8.8 Hz, 1H), 7.98 (d, J=2.0 Hz, 1H).

### Step B: Preparation of 1-Oxo-2,3-dihydro-1H-pyrrolo[1,2-a]indole-7-carbonitrile

To a suspension of 7-bromo-2,3-dihydro-1H-pyrrolo[1,2-a]indol-1-one (6.7 g, 26.8 mmol) in N-methylpyrrolidinone (20 mL) was added CuCN (3.12 g, 34.8 mmol). The mixture was heated under microwave irradiation in a sealed, thick-walled glass tube at 200° C. for 2 h. The reaction was diluted with dichloromethane (400 mL). Celite® was added and the mixture was stirred vigorously for 5 min. The solids were removed by filtration. The dichloromethane was removed under reduced pressure. The remaining brown liquid was poured into water (800 mL). The precipitate was collected by filtration, rinsed with water, and dried under reduced pressure to give the title compound as a beige solid (5.16 g). LCMS m/z=197.0 [M+H]⁺.

### Step C: Preparation of tert-Butyl 2-(7-Cyano-2,3-dihydro-1H-pyrrolo[1,2-a]indol-1-ylidene)acetate

To a solution of 1-oxo-2,3-dihydro-1H-pyrrolo[1,2-a]indole-7-carbonitrile (3.30 g, 16.82 mmol) in THF (50 mL) was added (tert-butoxycarbonylmethylene)triphenylphosphorane (15.83 g, 42.0 mmol). The mixture was stirred at 65° C. for 22 h and concentrated. The residue was purified by silica gel flash chromatography (20 to 75% EtOAc/hexanes) to provide the title compound as a pale yellow solid. LCMS m/z=295.1 [M+H]⁺.

### Step D: Preparation of tert-Butyl 2-(7-Cyano-2,3-dihydro-1H-pyrrolo[1,2-a]indol-1-yl)acetate

tert-Butyl 2-(7-cyano-2,3-dihydro-1H-pyrrolo[1,2-a]indol-1-ylidene)acetate (4.6 g, 15.63 mmol) was dissolved in EtOAc (100 mL). The flask was flushed with nitrogen gas. Degussa wet (50 wt % water) 10% Pd/C (1.663 g, 1.563 mmol) was added and the mixture was agitated on a Parr shaker under 70 psi hydrogen for 16 h. The mixture was filtered through Celite® and concentrated. To the residue was added further 10% Pd/C and the hydrogenation was repeated at 70 psi for 6 h. The reaction mixture was filtered and concentrated to provide the title compound as an orange solid. LCMS m/z=297.3 [M+H]⁺.

### Step E: Preparation of tert-Butyl 2-(7-(5-(3-Cyano-5-(trifluoromethoxy)phenyl)-1,2,4-oxadiazol-3-yl)-2,3-dihydro-1H-pyrrolo[1,2-a]indol-1-yl)acetate

To a solution of tert-butyl 2-(7-cyano-2,3-dihydro-1H-pyrrolo[1,2-a]indol-1-yl)acetate (4.95 g, 16.70 mmol) in ethanol (60 mL) was added a 50 wt % aqueous solution of hydroxylamine (10.24 mL, 167 mmol). The resulting solution was stirred in a sealed flask at 70° C. for 4 h. The solution was concentrated under reduced pressure to provide the title compound as a yellow solid. LCMS m/z=330.4 [M+H]⁺.

### Step F: Preparation of 3-Cyano-5-(trifluoromethoxy)benzoyl Chloride

To a solution of 3-cyano-5-(trifluoromethoxy)benzoic acid (11.0 g, 47.6 mmol) in dichloromethane (100 mL) was added DMF (10 drops). The solution was cooled in an ice-bath and a 2.0 M dichloromethane solution of oxalyl chloride (29.7 mL, 59.5 mmol) was added over 5 min. After addition was complete, the cooling bath was removed and the mixture was stirred at 23° C. for 4 h. The solvent was removed under reduced pressure to provide the title compound. ¹H NMR (400 MHz, CDCl₃) δ ppm 7.82 (s, 1H), 8.17 (s, 1H), 8.36 (s, 1H).

### Step G: Preparation of tert-Butyl 2-(7-(5-(3-Cyano-5-(trifluoromethoxy)phenyl)-1,2,4-oxadiazol-3-yl)-2,3-dihydro-1H-pyrrolo[1,2-a]indol-1-yl)acetate

To a solution of tert-butyl 2-(7-(N′-hydroxycarbamimidoyl)-2,3-dihydro-1H-pyrrolo[1,2-a]indol-1-yl)acetate (7.95 g, 24.14 mmol) in dioxane (175 mL) was added 3-cyano-5-(trifluoromethoxy)benzoyl chloride (6.02 g, 24.14 mmol) and triethylamine (11.77 mL, 84 mmol). The resulting suspension was stirred at 75° C. for 3.5 h. The reaction mixture was concentrated under reduced pressure. The residue was dissolved in dichloromethane, washed with 0.5 M HCl (200 mL), and then brine. The organic layer was dried over MgSO₄, filtered, and concentrated under reduced pressure to provide a beige solid. Crystallization from dichloromethane/hexanes yielded the title compound as a white solid. LCMS m/z=525.5 [M+H]⁺; NMR (400 MHz, CDCl₃) δ ppm 1.50 (s, 9H), 2.29-2.38 (m, 1H), 2.56 (dd, J=15.7, 8.1 Hz, 1H), 2.77 (dd, J=15.9, 6.8 Hz, 1H), 2.88-2.96 (m, 1H), 3.76 (quintet, J=7.5 Hz, 1H), 4.06-4.12 (m, 1H), 4.18-4.23 (m, 1H), 6.29 (s, 1H), 7.34 (d, J=8.6 Hz, 1H); 7.72 (s, 1H), 7.92 (dd, J=8.6, 1.5 Hz, 1H), 8.31 (s, 1H), 8.39 (s, 1H), 8.47 (t, J=1.3 Hz, 1H).

### Step H: Preparation of 2-(7-(5-(3-Cyano-5-(trifluoromethoxy)phenyl)-1,2,4-oxadiazol-3-yl)-2,3-dihydro-1H-pyrrolo[1,2-a]indol-1-yl)acetic Acid (Compound 2)

To a solution of tert-butyl 2-(7-(5-(3-cyano-5-(trifluoromethoxy)phenyl)-1,2,4-oxadiazol-3-yl)-2,3-dihydro-1H-pyrrolo[1,2-a]indol-1-yl)acetate (0.55 g, 1.049 mmol) and thioanisole (1.232 mL, 10.49 mmol) in dichloromethane (5 mL) was added trifluoroacetic acid (3.12 mL, 41.9 mmol). The resulting suspension was stirred at 23° C. for 3 h. The volatiles were removed under reduced pressure. The residue was taken up in hexanes and stirred overnight. The resulting solid was collected by filtration and rinsed with hexanes to provide a racemic mixture of the title compound. LCMS m/z=469.3 [M+H]⁺; ¹H NMR (400 MHz, DMSO-d₆) δ ppm 2.23-2.32 (m, 1H), 2.63 (dd, J=16.4, 8.1 Hz, 1H), 2.77 (dd, J=16.7, 6.8 Hz, 1H), 2.78-2.87 (m, 1H), 3.65 (quintet, J=7.5 Hz, 1H), 4.05-4.11 (m, 1H), 4.21-4.27 (m, 1H), 6.31 (s, 1H), 7.50 (d, J=8.8 Hz, 1H), 7.80 (dd, J=8.6, 1.5 Hz, 1H), 8.27 (d, J=1.0 Hz, 1H), 8.43 (s, 2H), 8.66 (t, J=1.3 Hz, 1H), 12.39 (s, 1H).

Resolution via Chiral HPLC.
Column: normal phase preparative ChiralPak AD-H, 250×20 mm ID, 5 μm particle size
Eluent: 100% acetonitrile with 0.05% trifluoroacetic acid
Gradient: isocratic
Flow: 13 mL/min
Detector: 280 nm
Retention Times: 1^{st} enantiomer: 7.9 min; 2^{nd} enantiomer: 14.9 min.

### Example 1.26

### Preparation of 2-(7-(5-(3-(1H-Tetrazol-5-yl)-5-(trifluoromethoxy)phenyl)-1,2,4-oxadiazol-3-yl)-2,3-dihydro-1H-pyrrolo[1,2-a]indol-1-yl)acetic Acid (Compound 9)

To a solution of 2-(7-(5-(3-cyano-5-(trifluoromethoxy)phenyl)-1,2,4-oxadiazol-3-yl)-2,3-dihydro-1H-pyrrolo[1,2-a]indol-1-yl)acetic acid (50 mg, 0.107 mmol) in DMF (1 mL) was added sodium azide (13.88 mg, 0.213 mmol) and ammonium chloride (11.42 mg, 0.213 mmol). The resulting mixture was stirred at 90° C. for 2 h and concentrated under reduced pressure. The residue was purified by preparative HPLC to provide the title compound as a white solid (28 mg). LCMS m/z=512.2 [M+H]⁺; ¹H NMR (400 MHz, DMSO-d6) δ ppm 2.23-2.32 (m, 1H), 2.63 (dd, J=16.4, 7.8 Hz, 1H), 2.78 (dd, J=16.7, 7.1 Hz, 1H), 2.78-2.87 (m, 1H), 3.65 (quintet, J=7.5 Hz, 1H), 4.05-4.11 (m, 1H), 4.21-4.27 (m, 1H), 6.32 (s, 1H), 7.50 (d, J=8.6 Hz, 1H), 7.83 (dd, J=8.6, 1.5 Hz, 1H), 8.31 (d, J=1.0 Hz, 3H), 8.85 (s, 1H), 12.41 (bs, 1H).

### Example 1.27

### Preparation of 2-(7-(5-(3-Cyano-4-(trifluoromethoxy)phenyl)-1,2,4-oxadiazol-3-yl)-2,3-dihydro-1H-pyrrolo[1,2-a]indol-1-yl)acetic Acid (Compound 33)

### Step A: Preparation of 3-Bromo-4-(trifluoromethoxy)benzoic Acid

To a suspension of 4-(trifluoromethoxy)benzoic acid (1.00 g, 4.85 mmol) in nitromethane (20 mL) was added FeCl₃ (790 mg, 4.85 mmol). To the resulting solution was added bromine (0.25 mL, 4.85 mmol). The orange solution was stirred at 100° C. for 1 h. Ice water (100 mL) was added. The mixture was extracted twice with EtOAc (2×70 mL). The combined organic extracts were washed with brine, dried over MgSO₄, filtered, and concentrated under reduced pressure to provide an off-white solid. It was further purified by preparative HPLC to provide the title compound as a white solid (0.75 g). LCMS m/z=285.4 [M+H]⁺; ¹H NMR (400 MHz, DMSO-d₆) δ ppm 7.68 (dd, J=8.6, 1.5 Hz, 1H), 8.06 (dd, J=8.6, 2.0 Hz, 1H), 8.26 (d, J=2.0 Hz, 1H), 13.60 (s, 1H).

### Step B: Preparation of 3-Cyano-4-(trifluoromethoxy)benzoic Acid

To a solution of 3-bromo-4-(trifluoromethoxy)benzoic acid (350 mg, 1.228 mmol) in NMP (5 mL) was added CuCN (121 mg, 1.351 mmol). The resulting mixture was heated under microwave irradiation at 175° C. for 2 h. Another portion of CuCN (60 mg) was added and the reaction was heated under microwave irradiation at 200° C. for another 2 h. The reaction was diluted with 75 mL dichloromethane. Celite® was added and the mixture was stirred vigorously for 5 min. The solids were removed by filtration. The dichloromethane was removed under reduced pressure. The remaining brown solution was poured into water (75 mL). The mixture was extracted with EtOAc then 25% isopropanol/dichloromethane. The combined organic extracts were washed with brine, dried over MgSO₄, filtered, and concentrated under reduced pressure. The residue was purified by preparative HPLC to provide the title compound as a white solid (160 mg). LCMS m/z=232.2 [M+H]⁺; ¹H NMR (400 MHz, Acetonitrile-d₃) δ ppm 7.61 (s, 1H), 8.32 (s, 1H), 8.43 (s, 1H), 10.10 (s, 1H).

### Step C: Preparation of tert-Butyl 2-(7-(5-(3-Cyano-4-(trifluoromethoxy)phenyl)-1,2,4-oxadiazol-3-yl)-2,3-dihydro-1H-pyrrolo[1,2-a]indol-1-yl)acetate

3-Cyano-4-(trifluoromethoxy)benzoyl chloride was prepared by stirring a solution of 3-cyano-4-(trifluoromethoxy)benzoic acid, DMF (catalytic amount) and two equivalents of oxalyl chloride in dichloromethane at room temperature for 2 h, then concentrating under reduced pressure.

To a solution of tert-butyl 2-(7-(N′-hydroxycarbamimidoyl)-2,3-dihydro-1H-pyrrolo[1,2-a]indol-1-yl)acetate (66 mg, 0.200 mmol) and 3-cyano-4-(trifluoromethoxy)benzoyl chloride (50.0 mg, 0.200 mmol) in dioxane (1 mL) was added triethylamine (0.084 mL, 0.601 mmol). The resulting suspension was stirred at room temperature for 30 min, then at 90° C. for 2.5 h and then concentrated under reduced pressure. The reaction mixture was passed through a silica plug and concentrated. The residue was purified by silica gel flash chromatography (25 to 100% EtOAc/hexanes) to provide the title compound as a white solid (70 mg). LCMS m/z=525.4 [M+H]⁺.

### Step D: Preparation of 2-(7-(5-(3-cyano-4-(trifluoromethoxy)phenyl)-1,2,4-oxadiazol-3-yl)-2,3-dihydro-1H-pyrrolo[1,2-a]indol-1-yl)acetic acid (Compound 33)

To a solution of tert-butyl 2-(7-(5-(3-cyano-4-(trifluoromethoxy)phenyl)-1,2,4-oxadiazol-3-yl)-2,3-dihydro-1H-pyrrolo[1,2-a]indol-1-yl)acetate (70 mg, 0.133 mmol) and thioanisole (0.157 mL, 1.335 mmol) in dichloromethane (1 mL) was added trifluoroacetic acid (0.397 mL, 5.34 mmol). The resulting suspension was stirred at 23° C. for 3 h. The dichloromethane and trifluoroacetic acid were removed under reduced pressure. The resulting sludge was taken up in hexanes and stirred for 2 h. The resulting solid was collected by filtration, and rinsed with hexanes to give a light brown solid. It was further purified by preparative HPLC to provide a racemic mixture of the title compound as a white solid (35 mg). LCMS m/z=469.3 [M+H]⁺; ¹H NMR (400 MHz, DMSO-d₆) δ ppm 2.23-2.32 (m, 1H), 2.63 (dd, J=16.4, 8.1 Hz, 1H), 2.77 (dd, J=16.7, 6.8 Hz, 1H), 2.78-2.87 (m, 1H), 3.65 (quintet, J=7.5 Hz, 1H), 4.05-4.11 (m, 1H), 4.21-4.26 (m, 1H), 6.31 (s, 1H), 7.51 (d, J=8.3 Hz, 1H), 7.81 (dd, J=8.5, 1.5 Hz, 1H), 7.97 (dd, J=8.8, 1.4 Hz, 1H), 8.28 (s, 1H), 8.62 (dd, J=8.8, 2.2 Hz, 1H), 8.85 (d, J=2.2 Hz, 1H), 12.39 (s, 1H).

### Example 1.28

### Preparation of 2-(7-(5-(6-(Trifluoromethyl)pyridin-3-yl)-1,2,4-oxadiazol-3-yl)-2,3-dihydro-1H-pyrrolo[1,2-a]indol-1-yl)acetic Acid (Compound 34)

### Step A: Preparation of tert-Butyl 2-(7-(5-(6-(Trifluoromethyl)pyridin-3-yl)-1,2,4-oxadiazol-3-yl)-2,3-dihydro-1H-pyrrolo[1,2-a]indol-1-yl)acetate

To a solution of tert-butyl 2-(7-(N′-hydroxycarbamimidoyl)-2,3-dihydro-1H-pyrrolo[1,2-a]indol-1-yl)acetate (0.200 g, 0.607 mmol) and TEA (0.093 mL, 0.668 mmol) in THF (2 mL) at 0° C. was added 6-(trifluoromethyl)nicotinoyl chloride (0.097 mL, 0.668 mmol) and the mixture was heated to 50° C. for 16 h in a 20 mL vial. The solvent was removed under reduced pressure. The residue was diluted with EtOAc (10 mL), washed with water (3×10 mL), dried over MgSO₄, filtered, and concentrated to give the title compound as a tan solid (0.294 g) which was used without further purification. LCMS m/z=485.1 [M+H]⁺. ¹H NMR (400 MHz, DMSO-d₆) δ ppm 1.46 (s, 9H), 2.27 (dd, J=12.51, 8.21 Hz, 1H), 2.58-2.76 (m, 2H), 2.81 (m, 1H), 3.65 (m, 1H), 4.08 (m, 1H), 4.24 (m, 1H), 6.31 (s, 1H), 7.51 (d, J=8.59 Hz, 1H), 7.82 (dd, J=8.59, 1.52 Hz, 1H), 8.21 (d, J=7.83 Hz, 1H), 8.31 (d, J=1.26 Hz, 1H), 8.83 (dd, J=8.21, 1.89 Hz, 1H), 9.51 (s, 1H).

### Step B: Preparation of 2-(7-(5-(6-(Trifluoromethyl)pyridin-3-yl)-1,2,4-oxadiazol-3-yl)-2,3-dihydro-1H-pyrrolo[1,2-a]indol-1-yl)acetic Acid (Compound 34)

tert-Butyl 2-(7-(5-(6-(trifluoromethyl)pyridin-3-yl)-1,2,4-oxadiazol-3-yl)-2,3-dihydro-1H-pyrrolo[1,2-a]indol-1-yl)acetate (0.294 g, 0.607 mmol) was dissolved in a pre-mixed solution of DCM (10 mL), TFA (1.870 mL, 24.27 mmol) and thioanisole (0.718 mL, 6.07 mmol) at room temperature for 16 h in a 20 mL sealed scintillation vial. After 16 h, the reaction was diluted with anhydrous toluene and the solvent was removed under reduced pressure to give a slurry that was co-evaporated with toluene again to remove residual TFA and to give a solid. The solid was suspended in DCM and hexanes were added. The DCM was evaporated to give a suspension in hexanes. The resulting precipitate was collected, washed with hexanes (3×10 mL) and triturated with DCM. The solid was collected and dried in a vacuum oven to give the title compound as an off-white solid (0.100 g). LCMS m/z=429.3 [M+H]⁺. ¹H NMR (400 MHz, DMSO-d₆) δ ppm 2.27 (m, 1H), 2.57-2.70 (m, 1H), 2.71-2.90 (m, 2H), 3.64 (t, 1H), 4.03-4.15 (m, 1H), 4.24 (m, 1H), 6.32 (s, 1H), 7.51 (d, J=8.46 Hz, 1H), 7.82 (d, J=8.21 Hz, 1H), 8.21 (d, J=7.96 Hz, 1H), 8.30 (s, 1H), 8.82 (d, J=7.96 Hz, 1H), 9.51 (s, 1H), 12.38 (bs, 1H).

### Example 1.29

### Preparation of 2-(7-(5-(3-(Trifluoromethoxy)phenyl)-1,2,4-oxadiazol-3-yl)-2,3-dihydro-1H-pyrrolo[1,2-a]indol-1-yl)acetic Acid (Compound 19)

### Step A: Preparation of tert-Butyl 2-(7-(5-(3-(Trifluoromethoxy)phenyl)-1,2,4-oxadiazol-3-yl)-2,3-dihydro-1H-pyrrolo[1,2-a]indol-1-yl)acetate

To a solution of tert-butyl 2-(7-(N′-hydroxycarbamimidoyl)-2,3-dihydro-1H-pyrrolo[1,2-a]indol-1-yl)acetate (0.075 g, 0.228 mmol) in dioxane (2 mL) was added 3-(trifluoromethoxy)benzoyl chloride (0.037 mL, 0.228 mmol) and triethylamine (0.111 mL, 0.797 mmol). The resulting suspension was stirred at 50° C. for 1 h and at 100° C. for 2 h. The reaction mixture was concentrated and the residue was dissolved in dichloromethane. The organics were washed with 0.5 M HCl and brine, dried over MgSO₄, filtered, and concentrated to give the title compound as a beige solid (0.0784 g). LCMS m/z=500.6 [M+H]⁺.

### Step B: Preparation of 2-(7-(5-(3-(Trifluoromethoxy)phenyl)-1,2,4-oxadiazol-3-yl)-2,3-dihydro-1H-pyrrolo[1,2-a]indol-1-yl)acetic Acid (Compound 19)

To a solution of tert-butyl 2-(7-(5-(3-(trifluoromethoxy)phenyl)-1,2,4-oxadiazol-3-yl)-2,3-dihydro-1H-pyrrolo[1,2-a]indol-1-yl)acetate (0.0784 g, 0.157 mmol) and thioanisole (0.184 mL, 1.570 mmol) in dichloromethane (2 mL) was added trifluoroacetic acid (0.466 mL, 6.28 mmol). The resulting solution was stirred at room temperature overnight. Volatiles were removed under reduced pressure. The residue was taken up in hexanes and stirred overnight. The resulting solid was collected by filtration, and rinsed with hexanes to give a purple solid that was further purified by preparative LCMS to yield the title compound as a solid (12.3 mg). LCMS m/z=444.3 [M+H]⁺.

### Example 1.30

### Preparation of 2-(7-(5-(3-Cyanophenyl)-1,2,4-oxadiazol-3-yl)-2,3-dihydro-1H-pyrrolo[1,2-a]indol-1-yl)acetic Acid (Compound 20)

From tert-butyl 2-(7-(N′-hydroxycarbamimidoyl)-2,3-dihydro-1H-pyrrolo[1,2-a]indol-1-yl)acetate and 3-cyanobenzoyl chloride, using a similar method to the one described in Example 1.29, the title compound was obtained as a solid. LCMS m/z=385.2 [M+H]⁺.

### Example 1.31

### Preparation of 2-(7-(5-(2-Chloro-6-methylpyridin-4-yl)-1,2,4-oxadiazol-3-yl)-2,3-dihydro-1H-pyrrolo[1,2-a]indol-1-yl)acetic Acid (Compound 35)

### Step A: Preparation of tert-Butyl 2-(7-(5-(2-Chloro-6-methylpyridin-4-yl)-1,2,4-oxadiazol-3-yl)-2,3-dihydro-1H-pyrrolo[1,2-a]indol-1-yl)acetate

To a solution of tert-butyl 2-(7-(N′-hydroxycarbamimidoyl)-2,3-dihydro-1H-pyrrolo[1,2-a]indol-1-yl)acetate (0.075 g, 0.228 mmol) and triethylamine (0.111 mL, 0.797 mmol) in THF was added 2-chloro-6-methylisonicotinoyl chloride (0.032 mL, 0.228 mmol). The resulting suspension was stirred overnight. The mixture was concentrated and dissolved in DMF, then heated at 90° C. for 4 h. The solvent was removed and the residue was taken up in EtOAc. The organics were washed with 1 M HCl followed by brine (2×). The organic layer was dried over MgSO₄, filtered, and concentrated under reduced pressure. The residue was purified by silica gel to yield the title compound as a white solid (0.078 g). LCMS m/z=465.3 [M+H]⁺. ¹H NMR (400 MHz, CDCl₃) δ ppm 1.51 (s, 9H), 2.29-2.39 (m, 1H), 2.57 (dd, J=15.79, 8.21 Hz, 1H), 2.69 (s, 3H), 2.78 (dd, J=15.92, 6.82 Hz, 1H), 2.93 (m, 1H), 3.77 (m, 1H), 4.09 (dt, J=10.10, 7.58 Hz, 1H), 4.21 (m, 1H), 6.29 (s, 1H), 7.34 (d, J=8.34 Hz, 1H), 7.87 (s, 1H), 7.91-7.97 (m, 2H), 8.40 (d, J=1.52 Hz, 1H).

### Step B: Preparation of 2-(7-(5-(2-Chloro-6-methylpyridin-4-yl)-1,2,4-oxadiazol-3-yl)-2,3-dihydro-1H-pyrrolo[1,2-a]indol-1-yl)acetic Acid (Compound 35)

From tert-butyl 2-(7-(5-(2-chloro-6-methylpyridin-4-yl)-1,2,4-oxadiazol-3-yl)-2,3-dihydro-1H-pyrrolo[1,2-a]indol-1-yl)acetate, using a similar method to the one described in Example 1.28, Step B, the title compound was obtained. LCMS m/z=409.3 [M+H]⁺. ¹H NMR (400 MHz, DMSO-d₆) δ ppm 2.22-2.33 (m, 1H), 2.58-2.68 (m, 4H), 2.73-2.87 (m, 2H), 3.65 (quintet, J=7.45 Hz, 1H), 4.08 (ddd, J=10.23, 7.71, 7.58 Hz, 1H), 4.24 (ddd, J=10.29, 8.53, 4.17 Hz, 1H), 6.31 (s, 1H), 7.51 (d, J=8.59 Hz, 1H), 7.81 (dd, J=8.59, 1.52 Hz, 1H), 7.99 (s, 1H), 8.04 (s, 1H), 8.28 (d, J=1.26 Hz, 1H).

### Example 1.32

### Preparation of 2-(7-(5-(3,5-Bis(trifluoromethyl)phenyl)-1,2,4-oxadiazol-3-yl)-2,3-dihydro-1H-pyrrolo[1,2-a]indol-1-yl)acetic Acid (Compound 1)

### Step A: Preparation of Methyl 2-(7-(5-(3,5-Bis(trifluoromethyl)phenyl)-1,2,4-oxadiazol-3-yl)-2,3-dihydro-1H-pyrrolo[1,2-a]indol-1-yl)acetate

From methyl 2-(7-(N′-hydroxycarbamimidoyl)-2,3-dihydro-1H-pyrrolo[1,2-a]indol-1-yl)acetate and 3,5-bis(trifluoromethyl)benzoyl chloride, using a similar method to the one described in Example 1.28, Step A, the title compound was obtained as a beige solid. LCMS m/z=510.5.

### Step B: Preparation of 2-(7-(5-(3,5-Bis(trifluoromethyl)phenyl)-1,2,4-oxadiazol-3-yl)-2,3-dihydro-1H-pyrrolo[1,2-a]indol-1-yl)acetic Acid

To a mixture of methyl 2-(7-(5-(3,5-bis(trifluoromethyl)phenyl)-1,2,4-oxadiazol-3-yl)-2,3-dihydro-1H-pyrrolo[1,2-a]indol-1-yl)acetate (0.020 g, 0.039 mmol) in 1,4-dioxane (1.60 mL, 18.70 mmol) and water (0.24 mL) was added lithium hydroxide (1.00 N, 0.157 mL, 0.157 mmol) dropwise at room temperature. The reaction was heated at 100° C. for 1 h. The mixture was cooled to room temperature and added HCl (1.0 N, 0.236 mL, 0.236 mmol) dropwise to adjust to pH 1. The mixture was diluted with EtOAc (50 mL). The organic layer was washed with water, brine, dried with MgSO₄, and the solvent was evaporated in vacuo to give the title compound (0.019 g) as a solid. LCMS m/z=496.3 [M+H]⁺; ¹H NMR (400 MHz, DMSO-d₆) δ ppm 2.18-2.36 (m, 1H), 2.57-2.69 (m, 1H), 2.77-2.89 (m, 2H), 3.61-3.71 (m, 1H), 4.03-4.15 (m, 1H), 4.17-4.34 (m, 1H), 6.32 (s, 1H), 7.50 (d, J=8.59 Hz, 1H), 7.84 (dd, J=8.59, 1.52 Hz, 1H), 8.33 (d, J=1.01 Hz, 1H), 8.55 (s, 1H), 8.74 (s, 2H), 12.37 (bs, 1H).

### Example 1.33

### Preparation of 2-(7-(5-(3-Cyano-4-fluorophenyl)-1,2,4-oxadiazol-3-yl)-2,3-dihydro-1H-pyrrolo[1,2-a]indol-1-yl)acetic Acid (Compound 30)

From tert-butyl 2-(7-(N′-hydroxycarbamimidoyl)-2,3-dihydro-1H-pyrrolo[1,2-a]indol-1-yl)acetate and 3-cyano-4-fluorobenzoyl chloride, using a similar method to the one described in Example 1.4, the title compound was obtained as a white solid. LCMS m/z=403.4 [M+H]⁺; ¹H NMR (400 MHz, DMSO-d₆) δ ppm 2.20-2.35 (m, 1H), 2.58-2.67 (m, 1H), 2.71-2.89 (m, 2H), 3.59-3.71 (m, 1H), 4.04-4.13 (m, 1H), 4.18-4.29 (m, 1H), 6.31 (s, 1H), 7.50 (d, J=8.59 Hz, 1H), 7.75-7.88 (m, 2H), 8.28 (s, 1H), 8.50-8.63 (m, 1H), 8.76 (dd, J=6.06, 2.02 Hz, 1H), 12.37 (s, 1H).

### Example 1.34

### Preparation of 2-(7-(5-(3-Cyano-4-hydroxyphenyl)-1,2,4-oxadiazol-3-yl)-2,3-dihydro-1H-pyrrolo[1,2-a]indol-1-yl)acetic Acid (Compound 31)

### Step A: Preparation of tert-Butyl 2-(7-(5-(3-Cyano-4-hydroxyphenyl)-1,2,4-oxadiazol-3-yl)-2,3-dihydro-1H-pyrrolo[1,2-a]indol-1-yl)acetate

To a solution of tert-butyl 2-(7-(5-(3-cyano-4-fluorophenyl)-1,2,4-oxadiazol-3-yl)-2,3-dihydro-1H-pyrrolo[1,2-a]indol-1-yl)acetate (0.217 g, 0.473 mmol) and isopropanol (0.073 mL, 0.947 mmol) in dioxane (0.947 mL) was added NaH (60%, 0.028 g, 0.710 mmol). The reaction mixture was stirred for 10 min and heated to 100° C. under microwave conditions for 30 min. It was then diluted with water and extracted with EtOAc (3×). The combined extracts were washed with brine, dried over MgSO₄ and concentrated. The residue was purified by column chromatography (1:3 EtOAc/hexanes) to provide the title compound as a yellow solid (36 mg). LCMS m/z=457.3 [M+H]⁺.

### Step B: Preparation of 2-(7-(5-(3-Cyano-4-hydroxyphenyl)-1,2,4-oxadiazol-3-yl)-2,3-dihydro-1H-pyrrolo[1,2-a]indol-1-yl)acetic Acid (Compound 31)

From tert-butyl 2-(7-(5-(3-cyano-4-hydroxyphenyl)-1,2,4-oxadiazol-3-yl)-2,3-dihydro-1H-pyrrolo[1,2-a]indol-1-yl)acetate, using a similar method to the one described in Example 1.5, Step B, the title compound was obtained as a pink solid. LCMS m/z=401.1 [M+H]⁺; ¹H NMR (400 MHz, DMSO-d₆) δ ppm 2.23-2.32 (m, 1H), 2.62 (dd, J=16.29, 7.96 Hz, 1H), 2.71-2.89 (m, 2H), 3.59-3.71 (m, 1H), 4.04-4.13 (m, 1H), 4.19-4.28 (m, 1H), 6.30 (s, 1H), 7.26 (d, J=8.84 Hz, 1H), 7.49 (d, J=8.34 Hz, 1H), 7.78 (dd, J=8.59, 1.52 Hz, 1H), 8.25 (d, J=1.26 Hz, 1H), 8.30 (dd, J=8.72, 2.15 Hz, 1H), 8.43 (d, J=2.27 Hz, 1H), 12.26 (s, 1H), 12.35 (s, 1H).

### Example 1.35

### Preparation of 2-(7-(5-(3-Cyano-4-methoxyphenyl)-1,2,4-oxadiazol-3-yl)-2,3-dihydro-1H-pyrrolo[1,2-a]indol-1-yl)acetic Acid (Compound 32)

From tert-butyl 2-(7-(N′-hydroxycarbamimidoyl)-2,3-dihydro-1H-pyrrolo[1,2-a]indol-1-yl)acetate and 3-cyano-4-methoxybenzoic acid, using a similar method to the one described in Example 1.1, Step A, the title compound was obtained as a white solid. ¹H NMR (400 MHz, CDCl₃) δ ppm 1.50 (s, 9H), 2.28-2.38 (m, 1H), 2.55 (dd, J=16.04, 8.21 Hz, 1H), 2.77 (dd, J=15.79, 6.69 Hz, 1H), 2.86-2.97 (m, 1H), 3.70-3.80 (m, 1H), 4.06 (s, 3H), 4.06-4.13 (m, 1H), 4.15-4.24 (m, 1H), 6.28 (s, 1H), 7.15 (d, J=9.09 Hz, 1H), 7.33 (d, J=8.59 Hz, 1H), 7.93 (dd, J=8.59, 1.52 Hz, 1H), 8.36-8.43 (m, 2H), 8.47 (d, J=2.27 Hz, 1H).

### Step B: Preparation of 2-(7-(5-(3-Cyano-4-methoxyphenyl)-1,2,4-oxadiazol-3-yl)-2,3-dihydro-1H-pyrrolo[1,2-a]indol-1-yl)acetic Acid (Compound 32)

To a solution of tert-butyl 2-(7-(5-(3-cyano-4-methoxyphenyl)-1,2,4-oxadiazol-3-yl)-2,3-dihydro-1H-pyrrolo[1,2-a]indol-1-yl)acetate (0.431 g, 0.916 mmol) in CH₂Cl₂ (3.05 mL) was added triisopropylsilane (1.884 mL, 9.16 mmol) and TFA (3.53 mL, 45.8 mmol). The mixture was stirred overnight. The solvent was removed under reduced pressure. The residue was triturated in EtOAc to give a grey solid. LCMS m/z=415.4 [M+H]⁺; ¹H NMR (400 MHz, DMSO-d₆) δ ppm 2.20-2.30 (m, 1H), 2.58-2.66 (m, 1H), 2.71-2.88 (m, 2H), 3.60-3.71 (m, 1H), 4.02-4.13 (m, 1H), 4.06 (s, 3H), 4.18-4.28 (m, 1H), 6.30 (s, 1H), 7.49 (d, J=8.59 Hz, 1H), 7.54 (d, J=9.09 Hz, 1H), 7.80 (dd, J=8.59, 1.52 Hz, 1H), 8.27 (d, J=1.52 Hz, 1H), 8.47 (dd, J=8.97, 2.15 Hz, 1H), 8.53 (d, J=2.02 Hz, 1H), 12.36 (s, 1H).

Resolution via Chiral HPLC.
Column: normal phase preparative ChiralPak AD-H, 250×20 mm ID, 5 μm particle size
Eluent: 100% acetonitrile with 0.05% trifluoroacetic acid
Gradient: isocratic
Flow: 30 mL/min
Detector: 254 nM
Retention Times: 1^{st} enantiomer: 10.3 min.; 2^{nd} enantiomer: 18.0 min.

### Example 1.36

### Preparation of 2-(7-(5-(3-Cyano-4-(1,1,1-trifluoropropan-2-yloxy)phenyl)-1,2,4-oxadiazol-3-yl)-2,3-dihydro-1H-pyrrolo[1,2-a]indol-1-yl)acetic Acid (Compound 36)

To a solution of tert-butyl 2-(7-(5-(3-cyano-4-fluorophenyl)-1,2,4-oxadiazol-3-yl)-2,3-dihydro-1H-pyrrolo[1,2-a]indol-1-yl)acetate (50 mg, 0.109 mmol) and 1,1,1-trifluoropropan-2-ol (0.040 mL, 0.436 mmol) in THF (1.0 mL) was added sodium hydride (60% dispersion in mineral oil, 17.45 mg, 0.436 mmol) resulting in vigorous gas evolution. After stirring at room temperature for 10 min, the solution was heated under microwave irradiation in a sealed, thick-walled glass tube at 110° C. for 60 min. HCl (4 M in dioxane, 1 mL) was added and the mixture was concentrated. The residue was purified by preparative HPLC to provide the title compound as a white solid (20 mg) (mixture of diastereomers). LCMS m/z=497.4 [M+H]⁺; ¹H NMR (400 MHz, DMSO-d₆) δ ppm 1.56 (d, J=6.44 Hz; 3H), 2.24-2.33 (m, 1H), 2.63 (dd, J=16.3, 7.8 Hz, 1H), 2.76 (dd, J=16.2, 6.7 Hz, 1H), 2.78-2.87 (m, 1H), 3.65 (quintet, J=7.5 Hz, 1H), 4.05-4.11 (m, 1H), 4.21-4.27 (m, 1H), 5.65-5.75 (m, 1H), 6.30 (s, 1H), 7.49 (d, J=8.5 Hz, 1H), 7.77 (d, J=9.5 Hz, 1H), 7.80 (dd, J=8.5, 1.6 Hz, 1H), 8.27 (d, J=1.5 Hz, 1H), 8.48 (dd, J=9.0, 2.3 Hz, 1H), 8.56 (d, J=2.3 Hz, 1H), 12.31 (bs, 1H).

### Example 1.37

### Preparation of 2-(7-(5-(2,6-Dichloropyridin-4-yl)-1,2,4-oxadiazol-3-yl)-2,3-dihydro-1H-pyrrolo[1,2-a]indol-1-yl)acetic Acid (Compound 37)

From tert-butyl 2-(7-(N′-hydroxycarbamimidoyl)-2,3-dihydro-1H-pyrrolo[1,2-a]indol-1-yl)acetate and 2,6-dichloroisonicotinoyl chloride, using a similar method to the one described in Example 1.31, the title compound was obtained. LCMS m/z=429.3 [M+H]⁺; ¹H NMR (400 MHz, DMSO-d₆) δ ppm 2.28 (dd, J=12.19, 7.64 Hz, 1H), 2.58-2.66 (m, 1H), 2.72-2.88 (m, 2H), 3.66 (quintet, J=7.55 Hz, 1H), 4.08 (dt, J=10.23, 7.58 Hz, 1H), 4.24 (m, 1H), 6.31 (s, 1H), 7.50 (d, J=8.46 Hz, 1H), 7.81 (dd, J=8.53, 1.58 Hz, 1H), 8.23 (s, 2H), 8.29 (d, J=1.26 Hz, 1H), 12.33 (bs, 1H).

### Example 1.38

### Preparation of 2-(7-(5-(5-isopropoxypyrazin-2-yl)-1,2,4-oxadiazol-3-yl)-2,3-dihydro-1H-pyrrolo[1,2-a]indol-1-yl)acetic acid (Compound 38)

### Step A. Preparation of tert-butyl 2-(7-(5-(5-chloropyrazin-2-yl)-1,2,4-oxadiazol-3-yl)-2,3-dihydro-1H-pyrrolo[1,2-a]indol-1-yl)acetate

tert-Butyl 2-(7-(N-hydroxycarbamimidoyl)-2,3-dihydro-1H-pyrrolo [1,2-a]indol-1-yl)acetate (0.700 g, 2.125 mmol) and triethylamine (0.884 mL, 6.38 mmol) were dissolved in dioxane (20 mL). To this solution at 0° C. was added 5-chloropyrazine-2-carbonyl chloride (0.752 g, 4.25 mmol) in dioxane (5 mL). The reaction mixture was stirred at room temperature for 1 h. After removal of dioxane under reduced pressure, the residue was dissolved in DMA. The solution was heated at 80° C. for 1 h. DMA was removed under reduced pressure. The residue was treated with water and extracted with ethyl acetate. The extract was dried over magnesium sulfate and concentrated to provide a brown crude which was purified by flash column chromatography to provide the title compound as a yellowish solid (0.400 g). LCMS m/z=452.2 (M+H⁺).

### Step B. Preparation of tert-Butyl 2-(7-(5-(5-Isopropoxypyrazin-2-yl)-1,2,4-oxadiazol-3-yl)-2,3-dihydro-1H-pyrrolo[1,2-a]indol-1-yl)acetate

tert-Butyl 2-(7-(5-(5-chloropyrazin-2-yl)-1,2,4-oxadiazol-3-yl)-2,3-dihydro-1H-pyrrolo[1,2-a]indol-2-yl)acetate (0.400 g, 0.885 mmol) and propan-2-ol (0.678 mL, 8.85 mmol) were dissolved in DMF (8 mL). Potassium 2-methylpropan-2-olate (1.018 mL, 1.018 mmol) was added slowly at 0° C. followed by cold water. The mixture was extracted with dichloromethane. The combined organic layers were dried and concentrated. The residue was purified by preparative HPLC. Fractions containing desired product were combined, partially concentrated, treated with saturated NaHCO₃. The organic layer was separated, dried, and concentrated to give the title compound as a yellow solid (0.262 g). LCMS m/z=476.5 (M+H⁺).

### Step C. Preparation of 2-(7-(5-(5-isopropoxypyrazin-2-yl)-1,2,4-oxadiazol-3-yl)-2,3-dihydro-1H-pyrrolo[1,2-a]indol-1-yl)acetic acid (Compound 38)

From tert-butyl 2-(7-(5-(5-isopropoxypyrazin-2-yl)-1,2,4-oxadiazol-3-yl)-2,3-dihydro-1H-pyrrolo[1,2-a]indol-1-yl)acetate, using a similar method to the one that was described in Example 1.27, Step B, the title compound was obtained. LCMS m/z=420.3 (M+H⁺). ¹H NMR (400 MHz, DMSO-d₆) δ ppm 1.36-1.42 (d, 6H), 2.22-2.32 (m, 1H), 2.39-2.47 (m, 1H), 2.73-2.88 (m, 2H), 3.61-3.70 (m, 1H), 4.03-4.12 (m, 1H), 4.19-4.17 (m, 1H), 5.33-5.44 (m, 1H), 6.31 (s, 1H), 7.49 (d, J=8.21 Hz, 1H), 7.80 (dd, J=8.46 Hz, 1.52 Hz, 1H), 8.27 (s, 1H), 8.46 (s, 1H), 9.08 (s, 1H), 12.33 (bs, 1H).

### Example 1.39

### Preparation of 2-(7-(5-(3-Cyano-5-(trifluoromethoxy)phenyl)-1,2,4-oxadiazol-3-yl)-2,3-dihydro-1H-pyrrolo[1,2-a]indol-1-yl)acetic Acid (Compound 2)

### Step A: Preparation of tert-Butyl 2-(7-(5-(3-Cyano-5-(trifluoromethoxy)phenyl)-1,2,4-oxadiazol-3-yl)-2,3-dihydro-1H-pyrrolo[1,2-a]indol-1-yl)acetate

In a 2 L flask equipped with a stirrer and thermocouple were placed 3-cyano-5-(trifluoromethoxy)benzoic acid (35 g, 163 mmol) and CDI (25 g, 154 mmol) in acetonitrile (400 mL) at room temperature. The mixture was heated to 60° C., cooled to 45° C. to add tert-butyl 2-(7-(N′-hydroxycarbamimidoyl)-2,3-dihydro-1H-pyrrolo[1,2-a]indol-1-yl)acetate (45.8 g, 139 mmol) and heated to 60° C. (bath). 2-Pyridinol (0.5 g, 5.26 mmol) was then added. The reaction was heated at 80° C. for 70 min until the starting materials were consumed. The mixture was cooled down in an ice bath, filtered and dried to give the title compound (43.8 g). LCMS m/z=525.5 [M+H]⁺.

### Step B: Preparation of 2-(7-(5-(3-Cyano-5-(trifluoromethoxy)phenyl)-1,2,4-oxadiazol-3-yl)-2,3-dihydro-1H-pyrrolo[1,2-a]indol-1-yl)acetic Acid

In a 1 L three neck flask equipped with stirrer, thermocouple and nitrogen inlet, was placed TFA (220 mL) and 2-amino-3-mercaptopropanoic acid (18.4 g, 152 mmol). tert-Butyl 2-(7-(5-(3-cyano-5-(trifluoromethoxy)phenyl)-1,2,4-oxadiazol-3-yl)-2,3-dihydro -1H-pyrrolo[1,2-a]indol-1-yl)acetate (41 g, 78 mmol) was added in portions at room temperature. The reaction was stirred at room temperature for 2 h and concentrated in vacuo. The residue was poured into ice water (1 L). The solid was collected by filtration, washed with water (1L) and suspended in dichloromethane (3 L). After the filtration, the insoluble material was washed with acetonitrile and concentrated to a solid compound (22 g). The dichloromethane layer washed with water (20 mL), dried over magnesium sulfate, filtered and concentrated to give a white powder (18 g). The isolated materials (18 g and 22 g) were combined, triturated with dichloromethane (200 mL), filtered and dried to give the title compound (32.6 g). LCMS m/z=469.3 [M+H]⁺.

### Example 2

### Homogeneous Time-Resolved Fluorescence (HTRF®) Assay For Direct cAMP Measurement

Compounds were screened for S1P1 receptor agonism (e.g., human S1P1 receptor) using the HTRF® assay for direct cAMP measurement (Gabriel et al, ASSAY and Drug Development Technologies, 1:291-303, 2003) and recombinant CHO-K1 cells stably transfected with S1P1 receptor. CHO-K1 cells were obtained from ATCC® (Manassas, Va.; Catalog #CCL-61). An agonist of the S1P1 receptor was detected in the HTRF® assay for direct cAMP measurement as a compound which decreased cAMP concentration. The HTRF® assay also was used to determine EC₅₀ values for S1P1 receptor agonists.

Principle of the assay: The HTRF® assay kit was purchased from Cisbio-US, Inc. (Bedford, Mass.; Catalog #62AM4PEC). The HTRF® assay supported by the kit is a competitive immunoassay between endogenous cAMP produced by the CHO-K1 cells and tracer cAMP labeled with the dye d2. The tracer binding is visualized by a monoclonal anti-cAMP antibody labeled with Cryptate. The specific signal (i.e., fluorescence resonance energy transfer, FRET) is inversely proportional to the concentration of unlabeled cAMP in the standard or sample.

Standard curve: The fluorescence ratio (665 nm/620 nm) of the standards (0.17 to 712 nM cAMP) included in the assay was calculated and used to generate a cAMP standard curve according to the kit manufacturer's instructions. The fluorescence ratio of the samples (test compound or compound buffer) was calculated and used to deduce respective cAMP concentrations by reference to the cAMP standard curve.

Setup of the assay: The HTRF® assay was carried out using a two-step protocol essentially according to the kit manufacturer's instructions, in 20 μl total volume per well, 384-well plate format (ProxiPlates; PerkinElmer, Fremont, Calif.; catalog #6008280). To each of the experimental wells was transferred 1500 recombinant CHO-K1 cells in 5 μl phosphate buffered saline containing calcium chloride and magnesium chloride (“PBS+”; Invitrogen, Carlsbad, Calif.; catalog #14040) supplemented with IBMX (250 μM) and rolipram (20 μM) (phosphodiesterase inhibitors; Sigma-Aldrich, St. Louis, Mo.; catalog #I5879 and catalog #R6520, respectively), followed by test compound in 5 μl compound buffer (PBS+ supplemented with 10 μl NKH477 (water-soluble forskolin derivative; SignaGen Laboratories, Gaithersburg, Md.; catalog #PKI-NKH477-010)) or 5 μl compound buffer. The plate was then incubated at room temperature for 1 hour. To each well was then added 5 μl cAMP-d2 conjugate in lysis buffer and 5 μl Cryptate conjugate in lysis buffer according to the kit manufacturer's instructions. The plate was then further incubated at room temperature for 1 hour, after which the assay plate was read.

Assay readout: HTRF® readout was accomplished using a PHERAstar (BMG LABTECH Inc., Durham, N.C.) or EnVision™ (PerkinElmer, Fremont, Calif.) microplate reader.

Certain compounds of the present invention and their corresponding activity values are shown in TABLE B.

TABLE B

| Compound No. | EC₅₀ S1P1 (HTRF) | |
|---|---|---|
| 1 | 7.2 | nM |
| 2* | 10 | nM |
| 18 | 13 | nM |
| 15 | 6.3 | nM |
| 8 | 41 | nM |

*Enantiomer 1 as described in Example 1.25 from chiral resolution.

Certain other compounds of the invention had activity values ranging from about 20 μM to about 0.5 nM in this assay.

### Example 3

### Cellular/Functional Ca²⁺ Assay for Agonist Activity on S1P3 Receptor

A compound of the invention can be shown to have no or substantially no agonist activity on the S1P3 receptor using in assay a human neuroblastoma cell line which endogenously expresses S1P3 (predominantly), S1P2 and S1P5 receptors, but not S1P1 or S1P4 receptors, based on mRNA analysis (Villullas et al, J Neurosci Res, 73:215-226, 2003). Of these, S1P3 and S1P2 receptors respond to agonists, such as S1P, with an intracellular calcium increase. No or substantially no increase of intracellular calcium in response to a test compound is indicative of the test compound exhibiting no or substantially no agonist activity on the S1P3 receptor. Such an assay can be performed commercially, e.g. by Caliper LifeSciences (Hopkinton, Mass.).

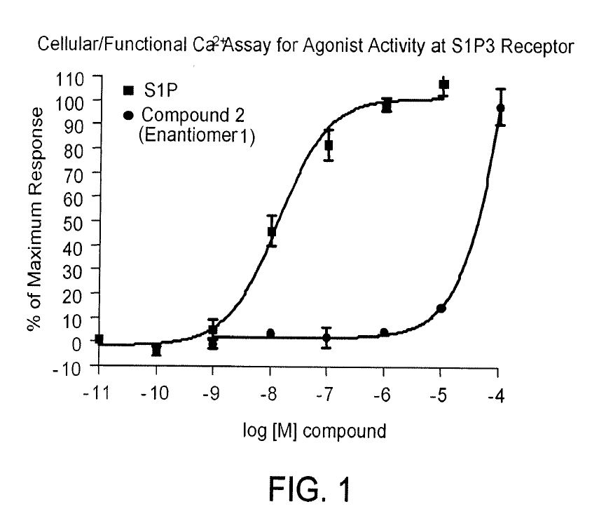

Assay: Compound 2 (Enantiomer 1) was assayed for agonist activity on S1P3 receptor by Caliper LifeSciences. Human neuroblastoma cells were washed and resuspended in physiological buffer. The cells were then loaded with dye that measures intracellular calcium. S1P was used as a reference agonist. Compound 2 (Enantiomer 1) was included in the assay as a test compound. After addition of S1P3 or Compound 2 (Enantiomer 1), fluorescence was measured at 485 nm excitation/525 nm emission every 2 sec for at least 60 sec. Calcium ionophore A23187 was then added as an internal positive control. Results are presented in FIG. 1. It is apparent from inspection of FIG. 1 that Compound 2 (Enantiomer 1) exhibited no or substantially no agonist activity on S1P3 receptor.

### Example 4

### Effect of Compounds in Peripheral Lymphocyte Lowering (PLL) Assay

A compound of the invention can be shown to induce peripheral lymphocyte lowering (PLL).

### A. Mouse PLL Assay

Animals: Male BALB/c mice (6 to 7 weeks of age at start of study) (Charles River Laboratories, Wilmington, Mass.) were housed four per cage and maintained in a humidity—(40-60%) and temperature—(68-72° F.) controlled facility on a 12 hr:12 hr light/dark cycle (lights on at 6:30 am) with free access to food (Harlan Teklad, Orange, Calif., Rodent Diet 8604) and water. Mice were allowed one week of habituation to the animal facility before testing.

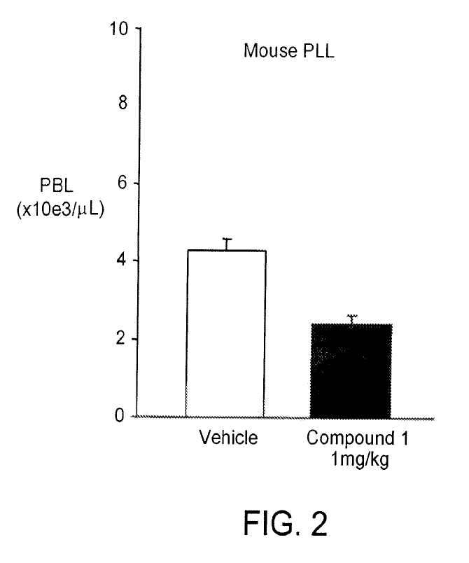

PLL Assay: Mice were administered via i.v. one of the following: vehicle (100% PEG400) or 1 mg/kg Compound 1 in vehicle, in a total volume of 5 mL/kg. Peripheral blood samples were collected at 5 hours post dose. The mice were anesthetized with isoflurane and bled retro-orbitally (300 μL) into 1.5 ml EDTA tubes. A complete cell count (CBC) (including lymphocyte count) was obtained using CELL-DYN® 3700 (Abbott Laboratories, Abbott Park, Ill.). Results are presented in FIG. 2, in which the peripheral blood lymphocyte (PBL) count is shown for the 5 hour group. Reduction of the peripheral blood lymphocyte count by the test compound in comparison with vehicle is indicative of the test compound exhibiting activity for inducing peripheral lymphocyte lowering. It is apparent from inspection of FIG. 2 that Compound 1 exhibited activity for inducing peripheral lymphocyte lowering (lymphopenia) in the mouse.

### B. Rat PLL Assay

Animals: Male Sprague-Dawley rats (7 weeks of age at start of study) (Charles River Laboratories) are housed two per cage and maintained in a humidity—(40-60%) and temperature—(68-72° F.) controlled facility on a 12 hr:12 hr light/dark cycle (lights on at 6:30 am) with free access to food (Harlan Teklad, Orange, Calif., Rodent Diet 8604) and water. Rats are allowed one week of habituation to the animal facility before testing.

PLL Assay: Rats are orally administered vehicle or test compound (e.g., 1 mg/kg, 3 mg/kg, 10 mg/kg or 30 mg/kg) in vehicle, in a total volume of 5 mL/kg. Peripheral blood samples are collected at 1, 3, 5, 8, 16 and 24 hours post dose. At each time point, the rats are anesthetized with isoflurane and bled retro-orbitally (200 μl) into 1.5 ml EDTA tubes. A complete cell count (CBC) (including lymphocyte count) is obtained using CELL-DYN® 3700 (Abbott Laboratories, Abbott Park, Ill.). Reduction of the peripheral blood lymphocyte count by the test compound in comparison with vehicle is indicative of the test compound exhibiting activity for inducing peripheral lymphocyte lowering.

### Example 5

### Effect of Compounds on Experimental Autoimmune Encephalomyelitis (EAE)

A compound of the invention can be shown to have therapeutic efficacy in multiple sclerosis by showing it to have therapeutic efficacy in experimental autoimmune encephalomyelitis (EAE), an animal model for multiple sclerosis. In certain exemplary well-established models, EAE is induced in rodents by injection of myelin oligodendrocyte glycoprotein (MOG) peptide, by injection of myelin basic protein (MBP) or by injection of proteolipid prbtein (PLP) peptide.

### A. MOG-Induced EAE in Mice

Animals: Female C57BL/6 mice (8 to 10 weeks of age at start of study) (Jackson Laboratory, Bar Harbor, Me.) were housed four per cage and maintained in a humidity—(40-60%) and temperature—(68-72° F.) controlled facility on a 12 hr:12 hr light/dark cycle (lights on at 6:30 am) with free access to food (Harlan Teklad, Orange, Calif., Rodent Diet 8604) and water. Mice were allowed one week of habituation to the animal facility before testing.

Induction of EAE: Mice were immunized subcutaneously, 50 μL per hind flank, with a total of 100 μg MOG₃₅₋₅₅ peptide emulsified 1:1 with Complete Freund's adjuvant containing 4 mg/mL heat-killed Mycobacterium tuberculosis. Mice also received 200 ng pertussis toxin intraperitoneally on the day of immunization and 48 hours later.

Clinical scoring: Severity of disease symptoms was scored as follows (in increasing order of severity): 0=normal; 1=limp tail OR hind limb weakness; 2=limp tail AND limb weakness/weakness of 2 or more limbs; 3=severe limb weakness or single limb paralysis; 4=paralysis of 2 or more limbs; 5=death.

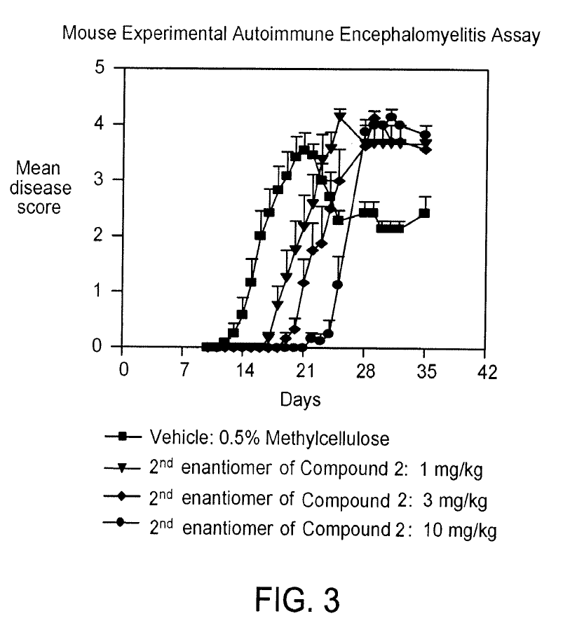

Drug treatment: Mice were dosed orally, with vehicle or test compound, once a day from day 3 until day 21. Dosing volume is 5 mL/kg. Test compound were dosed at 1 mg/kg, 3 mg/kg and 10 mg/kg. Mice were weighed daily. Mice were monitored daily from day 7 onward for disease symptoms. After the last dose on day 21, disease progression was monitored daily for 2 more weeks. Reduction of the severity of disease symptoms by treatment with the test compound in comparison with vehicle was indicative of the test compound exhibiting therapeutic efficacy in EAE. It is apparent from inspection of FIG. 3 that the 2^{nd} enantiomer of Compound 2 (isolated after resolution of compound 2 by HPLC, with a retention time of 14.9 min per the conditions reported in Example 1.25) exhibited activity in the mouse EAE assay.

### B. PLP-Induced EAE in Mice

Animals: Female SJL/J mice (8 to 10 weeks of age at start of study) (Jackson Laboratory, Bar Harbor, Me.) are housed four per cage and maintained in a humidity—(40-60%) and temperature—(68-72° F.) controlled facility on a 12 hr:12 hr light/dark cycle (lights on at 6:30 am) with free access to food (Harlan Teklad, Orange, Calif., Rodent Diet 8604) and water. Mice are allowed one week of habituation to the animal facility before testing.

Induction of EAE: Mice are immunized subcutaneously with 100 μg PLP₁₃₉₋₁₅₁ peptide emulsified 1:1 with Complete Freund's adjuvant containing 4 mg/mL heat-killed Mycobacterium tuberculosis. Mice also receive 200 ng pertussis toxin intravenously on the day of immunization.

Clinical scoring: Severity of disease symptoms is scored as follows (in increasing order of severity): 0=normal; 1=limp tail OR hind limb weakness; 2=limp tail AND limb weakness/weakness of 2 or more limbs; 3=severe limb weakness or single limb paralysis; 4=paralysis of 2 or more limbs; 5=death.

Drug treatment: Mice are dosed orally, with vehicle or test compound, once a day from day 3 until day 21. Dosing volume is 5 mL/kg. Test compound is dosed at, e.g., 1 mg/kg, 3 mg/kg, 10 mg/kg or 30 mg/kg. Mice are weighed daily. Mice are monitored daily from day 7 onward for disease symptoms. After the last dose on day 21, disease progression is monitored daily for 2 more weeks.

### C. MBP-Induced EAE in Rats

Animals: Male Lewis rats (325-375 g at start of study) (Harlan, San Diego, Calif.) are housed two per cage and maintained in a humidity—(30-70%) and temperature—(20-22° C.) controlled facility on a 12 hr:12 hr light/dark cycle (lights on at 6:30 A.M.) with free access to food (Harlan Teklad, Orange, Calif., Rodent Diet 8604) and water. Rats are allowed one week of habituation to the animal facility before testing. During the study, rats are weighed daily prior to clinical scoring at 11 am.

Induction of EAE: Myelin basic protein (MBP; guinea pig) is dissolved in sterile saline at a concentration of 1 mg/mL, and then emulsified 1:1 with Complete Freund's adjuvant (1 mg/mL). 50 μl of this emulsion is administered by intraplantar (ipl) injection into both hind paws of each rat, for a total injected volume of 100 μl per rat and a total dose of 50 μg of MBP per rat.

Clinical scoring: Severity of disease symptoms is scored daily after body weighing and before drug dosing. Severity of disease symptoms is scored as follows (in increasing order of severity): 0=normal; 1=tail OR limb weakness; 2=tail AND limb weakness; 3=severe hind limb weakness or single limb paralysis; 4=loss of tail tone and paralysis of 2 or more limbs; 5=death.

Drug treatment: Rats are dosed orally, with vehicle or test compound, 1 hr prior to MBP injection on day 0 and daily thereafter, after clinical scoring, for the duration of the study. Dosing volume is 5 mL/kg. Test compound is dosed at, e.g., 1 mg/kg, 3 mg/kg, 10 mg/kg or 30 mg/kg. Reduction of the severity of disease symptoms by treatment with the test compound in comparison with vehicle is indicative of the test compound exhibiting therapeutic efficacy in EAE.

### Example 6

### Effect of Compounds on Type 1 Diabetes

A compound of the invention can be shown to have therapeutic efficacy in type 1 diabetes using an animal model for type 1 diabetes, such as cyclophosphamide induced type 1 diabetes in mice.

Animals: Baseline blood glucose measurements were taken from 9-10 week old female NOD/Ltj mice (Jackson Laboratory, Bar Harbor, Me.) to ensure that they are normoglycemic (blood glucose is 80-120 mg/dL) prior to initiation of the experiment. Blood glucose was measured from tail bleeds using a One Touch Ultra® kit and test strips (LifeScan, Milpitas, Calif.).

Cyclophosphamide induction of Type 1 diabetes: On day 0 and day 14, normoglycemic NOD mice were injected intraperitoneally with 4 mg cyclophosphamide monohydrate (200 mg/kg) dissolved in 0.9% saline. If mice were diabetic (blood glucose is >250 mg/dL) they were not given a booster dose of cyclophosphamide on day 14.

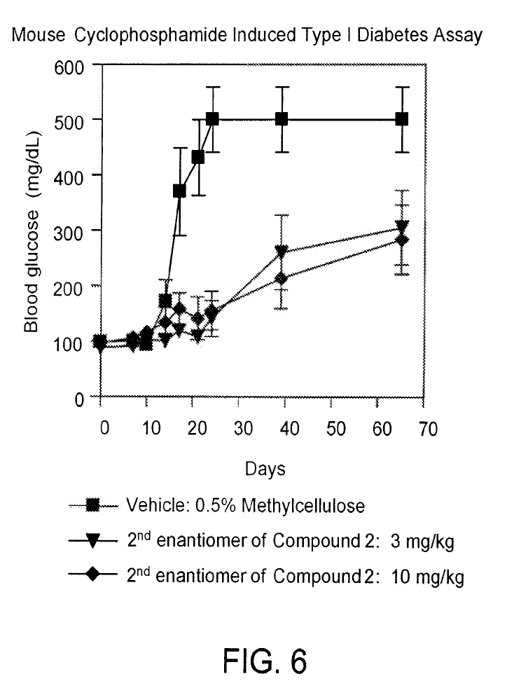

Drug Treatment: Mice were dosed orally, with vehicle or test compound, once a day from day 0 until day 25. Compounds were suspended in 0.5% methyl cellulose vehicle using a sonicator to ensure uniform suspension. Mice were weighed twice weekly and were dosed according to weight. Dosing volume is 5 mL/kg. Test compound was dosed at 3 mg/kg and 10 mg/kg. Blood glucose was measured twice weekly. After dosing was completed at day 25, the mice continued to be monitored and blood glucose measurements were taken once a week for 3 weeks. Promotion of normoglycemia by treatment with the test compound in comparison with vehicle was indicative of the test compound exhibiting therapeutic efficacy in Type 1 diabetes. It is apparent from inspection of FIG. 6 that the 2^{nd} enantiomer of Compound 2 (isolated after resolution of compound 2 by HPLC, with a retention time of 14.9 min per the conditions reported in Example 1.25) exhibited activity in the mouse type I diabetes assay.

### Example 7

### Allograft Survival

A compound of the invention can be shown to have therapeutic efficacy in prolonging allograft survival by showing it to have therapeutic efficacy in prolonging, e.g., survival of skin allograft in an animal model.

Animals: Female BALB/cJ mice (6 to 7 weeks of age at start of study) (Jackson Laboratory, Bar Harbor, Me.) were housed four per cage and maintained in a humidity—(40-60%) and temperature—(68-72° F.) controlled facility on a 12 hr:12 hr light/dark cycle (lights on at 6:30 am) with free access to food (Harlan Teklad, Orange, Calif., Rodent Diet 8604) and water. Female C57BL/6 mice (8 to 10 weeks of age at start of study) (Jackson Laboratory, Bar Harbor, Me.) were similarly housed and maintained. Mice were allowed one week of habituation to the animal facility before testing.

Skin allograft: BALB/cJ and C57BL/6 mice were used as donors and recipients, respectively, in a model of skin allograft transplantation. Donor BALB/cJ mice were anesthetized, and 0.5 cm—diameter full thickness areas of abdominal skin were surgically removed. Skin grafts harvested from the BALB/cJ mice were sutured onto the dorsum of anesthetized recipient C57BL/6 mice. Sutured allografts were covered with Vaseline gauze and Bolster dressing for 7 days. The allografted mice were divided into eight groups of 8 mice each.

Clinical scoring: Skin allografts were inspected and digital images recorded daily until rejection, which was defined as the first day on which more than 80% of the graft is necrotic. Histological analysis of the rejected graft was carried out on hematoxylin and eosin (H&E)-stained sections. In an optional related study, on posttransplantation day 5 isolated lymphocytes from peripheral lymph nodes and spleen were counted and characterized for activation markers (e.g., T-cell activation markers) by flow cytometry. Also on day 5, grafts were removed from transplanted recipients, cut into small fragments, digested with collagenase and sedimented over Ficoll-Paque (Pharmacia Biotech, Uppsala, Sweden) to isolate graft-infiltrating lymphocytes, which were counted and characterized for activation markers (e.g., T-cell activation markers) by flow cytometry. Histological analysis of the graft on day 5 was carried out on hematoxylin and eosin (H&E)-stained sections.

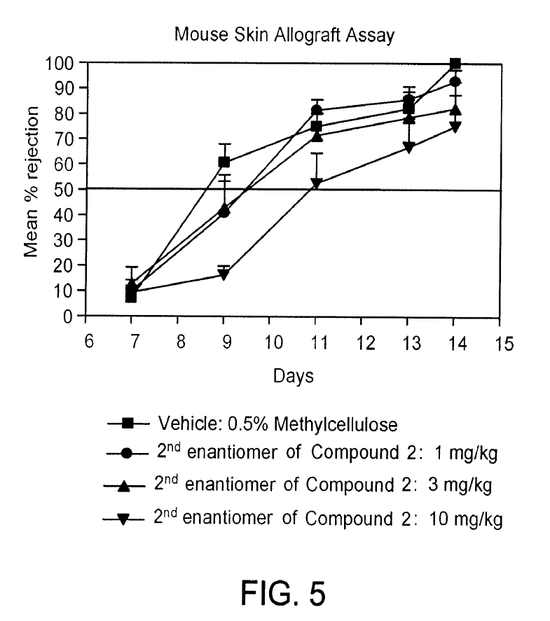

Drug treatment: Mice were dosed orally, with vehicle or test compound, once a day from the day of transplantation until the end of the study. Dosing volume is 5 mL/kg. Test compound was dosed at 1 mg/kg, 3 mg/kg and 10 mg/kg. Delay of time of rejection of the skin allograft by treatment with the test compound in comparison with vehicle was indicative of the test compound exhibiting therapeutic efficacy in prolonging skin allograft survival. It is apparent from inspection of FIG. 5 that the 2^{nd} enantiomer of Compound 2 (isolated after resolution of compound 2 by HPLC, with a retention time of 14.9 min per the conditions reported in Example 1.25) exhibited activity in the mouse skin allograft assay.

### Example 8

### Effect of Compounds on Colitis

A compound of the invention can be shown to have therapeutic efficacy in colitis using an animal model for colitis. Suitable animals models are known in the art (Boismenu et al, J Leukoc Biol, 67:267-278, 2000). A first exemplary animal model for colitis is trinitrobenzene sulfonic acid (TNBS)-induced colitis, which presents clinical and histopathological findings that resemble those in Crohn's disease (Neurath et al, J Exp Med, 182:1281-1290, 1995; Boismenu et al, J Leukoc Biol, 67:267-278, 2000). A second exemplary animal model for colitis is dextran sulfate sodium (DSS)-induced colitis, which presents clinical and histopathological findings that resemble those in ulcerative colitis (Okayasu et al, Gastroenterology, 98:694-702, 1990; Boismenu et al, J Leukoc Biol, 67:267-278, 2000). Compounds can be commercially tested for efficacy in at least DSS-induced colitis and TNBS-induced colitis, e.g. by Jackson Laboratory (Bar Harbor, Me.).

### A. Mouse Model for Colitis

Animals: Male BALB/c mice (6 weeks of age at start of study) (Jackson Laboratory, Bar Harbor, Me.) are housed four per cage and maintained in a humidity—(40-60%) and temperature—(68-72° F.) controlled facility on a 12 hr:12 hr light/dark cycle (lights on at 6:30 am) with free access to food (Harlan Teldad, Orange Calif., Rodent Diet 8604) and water. Mice are allowed one week of habituation to the animal facility before testing.

TNBS induction of colitis: Mice are weighed for baseline body weights and fasted later that day beginning at 6:15 pm just prior to lights-out (day 0). Body weights are taken again the following morning (day 1) at approximately 7:30 am. Mice are anesthetized with isoflurane prior to induction of colitis. Colitis is induced in the mice by intracolonic injection of about 150 mg/kg TNBS in 50% ethanol (in a volume of 150 μl) using an intubation needle (22 g, 1.5 in) inserted completely into the anus with the mouse held by the tail in a vertical position. The mouse is held vertically for a 30 additional sec to allow thorough absorption and minimize leakage, after which the mouse is returned to its cage. Mice are then fed, following the preceding approximately 14 hr of fasting. Each morning thereafter, the mice are weighed. In control experiments, mice receive 50% ethanol alone using the same protocol.

Drug treatment: Drug treatment begins on day 2. Mice are dosed orally, with vehicle or test compound, once a day from day 2 until the conclusion of the experiment on, e.g., day 7, 14 or 21. Dosing volume is 5 mL/kg. Test compound is dosed at, e.g., 1 mg/kg, 3 mg/kg, 10 mg/kg or 30 mg/kg.

Clinical scoring: Upon conclusion of the experiment, colons are extracted and measured. Mice are euthanized with CO₂ and colon is removed from anus to cecum. Excised colon is measured for entire length, length from anus to end of inflamed area, and length of inflamed (affected) area. After measurements, colon is cleared of excrement by flushing with saline and then cut open to clear more thoroughly. Colon is then weighed and preserved in neutral buffered formalin (NBF; 10% formalin, pH 6.7-7.0). The colon tissue is embedded in paraffin and processed for hematoxylin and eosin (H & E)-stained sections. Severity of disease symptoms is scored histologically from the stained sections as follows: 0=no evidence of inflammation; 1=low level of leukocyte infiltration with infiltration seen in <10% of high-power fields AND no structural changes; 2=moderate leukocyte infiltration with infiltration seen in 10% to 25% of high-power fields AND crypt elongation AND bowel wall thickening that does not extend beyond the mucosal layer AND no ulcerations; 3=high level of leukocyte infiltration seen in 25% to 50% of high-power fields AND crypt elongation AND infiltration beyond the mucosal layer AND thickening of the bowel wall AND superficial ulcerations; 4=marked degree of transmural leukocyte infiltration seen in >50% of high-power fields AND elongated and distorted crypts AND bowel-wall thickening AND extensive ulcerations. Reduction of the severity of the disease symptoms by treatment with the test compound in comparison with vehicle is indicative of the test compound exhibiting therapeutic efficacy in colitis.

### B. Rat Model for Colitis

Animals: Male Wistar rats (175-200 g at start of study) (Charles River Laboratories, Wilmington, Mass.) are housed two per cage and maintained in a humidity—(40-60%) and temperature—(68-72° F.) controlled facility on a 12 hr:12 hr light/dark cycle (lights on at 6:30 am) with free access to food (Harlan Teklad, Orange Calif., Rodent Diet 8604) and water. Rats are allowed one week of habituation to the animal facility before testing.

TNBS induction of colitis: Rats are weighed for baseline body weights and fasted later that day beginning at 6:15 pm just prior to lights-out (day 0). Body weights are taken again the following morning (day 1) at approximately 7:30 am. Rats are anesthetized with isoflurane prior to induction of colitis. Colitis is induced in the rats by intracolonic injection of about 60 mg/kg TNBS in 50% ethanol (in a volume of 500 μl) using a fabricated intubation needle (7.5 Fr umbilical catheter and 14 g hub) inserted 8 cm into the anus with the rat held by the tail in a vertical position. The rat is held vertically for 30 additional sec to allow thorough absorption and minimize leakage, after which the rat is returned to its cage. Rats are then fed, following the preceding approximately 14 hr of fasting. Each morning thereafter, the rats are weighed. In control experiments, rats receive 50% ethanol alone using the same protocol.

Drug treatment: Drug treatment begins on day 2. Rats are dosed orally, with vehicle or test compound, once a day from day 2 until the conclusion of the experiment on, e.g., day 7, 14 or 21. Dosing volume is 5 mL/kg. Test compound is dosed at, e.g., 1 mg/kg, 3 mg/kg, 10 mg/kg or 30 mg/kg.

Clinical scoring: Upon conclusion of the experiment, colons are extracted and measured. Rats are euthanized with CO₂ and colon is removed from anus to cecum. Excised colon is measured for entire length, length from anus to end of inflamed area, and length of inflamed (affected) area. After measurements, colon is cleared of excrement by flushing with saline and then cut open to clear more thoroughly. Colon is then weighed and preserved in neutral buffered formalin (NBF; 10% formalin, pH 6.7-7.0). The colon tissue is embedded in paraffin and processed for hematoxylin and eosin (H & E)-stained sections. Severity of disease symptoms is scored histologically from the stained sections as follows: 0=no evidence of inflammation; 1=low level of leukocyte infiltration with infiltration seen in <10% of high-power fields AND no structural changes; 2=moderate leukocyte infiltration with infiltration seen in 10% to 25% of high-power fields AND crypt elongation AND bowel wall thickening that does not extend beyond the mucosal layer AND no ulcerations; 3=high level of leukocyte infiltration seen in 25% to 50% of high-power fields AND crypt elongation AND infiltration beyond the mucosal layer AND thickening of the bowel wall AND superficial ulcerations; 4=marked degree of transmural leukocyte infiltration seen in >50% of high-power fields AND elongated and distorted crypts AND bowel-wall thickening AND extensive ulcerations. Reduction of the severity of the disease symptoms by treatment with the test compound in comparison with vehicle is indicative of the test compound exhibiting therapeutic efficacy in colitis.

### Example 9

### Effects of Compounds on Cardiac Telemetry in the Rat

Animals: Male Sprague-Dawley rats (250-300 g at time of surgery) are implanted by Charles River Laboratories (Wilmington, Mass.) with cardiac transmitting devices (Data Sciences PhysioTel C50-PXT) into the peritoneal space, with a pressure-sensing catheter inserted into the descending aorta. Rats are allowed at least one week to recover. Rats are housed in individual cages and maintained in a humidity—(30-70%) and temperature—(20-22° C.) controlled facility on a 12 hr:12 hr light/dark cycle (lights on at 7:00 am) with free access to food (Harlan-Teklad, Orange, Calif., Rodent Diet 8604) and water. Rats are allowed one week of habituation to the animal facility before testing.

Measurement of cardiovascular parameters: The implanted transmitting devices transmit continuous measurement of blood pressure (systolic, diastolic, mean arterial, pulse), heart rate, body temperature, and motor activity in freely moving conscious animals. These data are transmitted wirelessly to a computer which bins the data into 1-minute averages using DataSciences ART software. Telemetry recording occurred over a 21-hr period, starting at Noon and continuing until 9:00 am the following day. A maximum of eight rats are tested at a time, and the same eight rats are utilized for all treatment groups in a within-subject design.

Drug treatment: Rats are injected orally with vehicle or compound at 1:00 pm. A full study (vehicle+3 doses) requires 4 separate testing sessions, which occur on Mondays-Tuesdays and Thursdays-Fridays. During each testing session, the eight rats are divided into four treatment groups such that each group comprises N=2 for any given session. Rats are re-tested in subsequent test sessions in a crossover design such that by the end of the 4 sessions, all animals receive all treatments in a pseudo-random order, and each group comprises N=8.

Exemplary bradycardia assay: It is expressly contemplated that the rats can be used to show that a compound of the invention has no or substantially no activity for bradycardia. By way of illustration and not limitation, the rats are administered vehicle or test compound and heart rate is then measured over a 120 minute period. No or substantially no reduction of heart rate in response to the test compound in comparison with vehicle is indicative of the test compound exhibiting no or substantially no activity for bradycardia.

### Example 10

### Effect of Compounds on Arthritis

Female Lewis rats were used in this study. Acclimated animals were anesthetized with isoflurane and given the first collagen injection (day 0). On day 6, they were anesthetized again for the second collagen injection. Collagen was prepared by making a 4 mg/mL solution in 0.01 N acetic acid. Equal volumes of collagen and incomplete Freund's adjuvant were emulsified by hand mixing until a bead of this material held its form when placed in water. Each animal received 300 μL of the mixture each time, spread over 3 subcutaneous sites on the back.

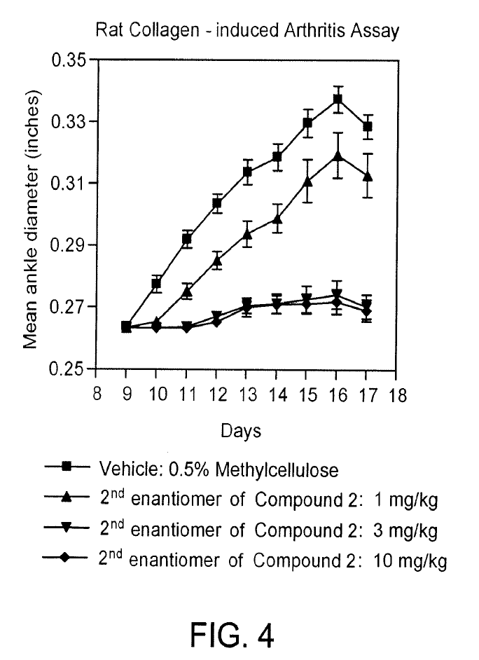

Treatment (p.o., q. d., 5 mL/kg dosing volume) began on day 0 and continued through day 16 with vehicle or compounds given at 24 h intervals. Rats were weighed on days 0, 3, 6 and 9 through 17 and caliper measurements of the ankles taken on days 9 through 17. The 2^{nd} enantiomer of Compound 2 (isolated after resolution of compound 2 by HPLC, with a retention time of 14.9 min per the conditions reported in Example 1.25) was dosed at 1, 3 and 10 mg/kg. Results are presented in FIG. 4. It is apparent from inspection of FIG. 4 that the 2^{nd} enantiomer of Compound 2 (isolated after resolution of compound 2 by HPLC, with a retention time of 14.9 min per the conditions reported in Example 1.25) exhibited activity for reducing mean ankle diameter in the rat.

Those skilled in the art will recognize that various modifications, additions, substitutions and variations to the illustrative examples set forth herein can be made without departing from the spirit of the invention and are, therefore, considered within the scope of the invention. All documents referenced above, including, but not limited to, printed publications and provisional and regular patent applications, are incorporated herein by reference in their entirety.

1. A compound selected from compounds of Formula (Ia) and pharmaceutically acceptable salts, solvates and hydrates thereof: 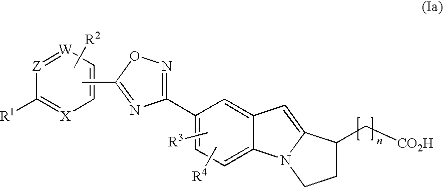
wherein:
n is 0 or 1;
W is N or CR⁵;
Z is N or CR⁶;
X is N or CR⁷; provided that W, Z and X are not all N;
R¹, R², R⁵, R⁶ and R⁷ are each independently selected from the group consisting of H, C₁-C₆ acyl, C₁-C₆ acyloxy, C₁-C₆ alkoxy, C₁-C₆ alkoxycarbonylamino, C₁-C₆ alkyl, C₂-C₆ alkynyl, C₁-C₆ alkylamino, C₂-C₈ dialkylamino, C₁-C₆ alkylcarboxamide, C₁-C₆ alkylsulfonamide, C₁-C₆ alkylsulfinyl, C₁-C₆ alkylsulfonyl, C₁-C₆ alkylthio, C₁-C₆ alkylureyl, amino, carbo-C₁-C₆-alkoxy, carboxamide, carboxy, cyano, C₃-C₇ cycloalkyl, C₃-C₇ cycloalkyloxy, C₃-C₇ cycloalkylthio, C₃-C₇ cycloalkylsulfinyl, C₃-C₇ cycloalkylsulfonyl, C₂-C₆ dialkylcarboxamide, C₁-C₆ haloalkoxy, C₁-C₆ haloalkyl, C₁-C₆ haloalkylsulfinyl, C₁-C₆haloalkylsulfonyl, halogen, heteroaryl, heterocyclyl, hydroxyl, nitro and sulfonamide, wherein C₁-C₆ alkoxy, C₁-C₆ alkylthio, C₁-C₆ alkyl, C₂-C₆ alkynyl and heteroaryl are optionally substituted with one substituent selected from the group consisting of C₁-C₆ alkoxy, carbo-C₁-C₆-alkoxy, cyano, C₃-C₇ cycloalkyl, halogen and phenyl, or
two adjacent groups selected from R¹, R², R⁵, R⁶ and R⁷ together with the atoms to which they are both bonded form a five or six member heterocyclyl ring optionally substituted with one or two halogen atoms; and
R³ and R⁴ are each independently selected from the group consisting of H, C₁-C₂ alkyl, fluoro and chloro.

2. The compound according to claim 1, wherein R³ and R⁴ are each independently selected from the group consisting of H, CH₃ and F.

3. The compound according to claim 1, wherein R³ and R⁴ are both H.

4. The compound according to claim 1, wherein n is 1.

5. The compound according to claim 1, wherein n is 0.

6. The compound according to claim 1, wherein R¹, R², R⁵, R⁶ and R⁷ are each independently selected from the group consisting of H, C₁-C₆ alkoxy, C₁-C₆ alkyl, C₁-C₆ alkylcarboxamide, cyano, C₁-C₆ haloalkoxy, C₁-C₆ haloalkyl, halogen, heteroaryl, heterocyclyl and hydroxyl, or
two adjacent groups selected from R¹, R², R⁵, R⁶ and R⁷ together with the atoms to which they are both bonded form a five member heterocyclyl ring optionally substituted with two halogen atoms.

7. The compound according to claim 1, wherein R¹, R², R⁵, R⁶ and R⁷ are each independently selected from the group consisting of H, methoxy, isopropoxy, methyl, acetamido, cyano, trifluoromethoxy, difluoromethoxy, 2,2,2-trifluoroethoxy, 1,1,1-trifluoropropan-2-yloxy, trifluoromethyl, fluoro, chloro, bromo, tetrazol-5-yl, pyrazol-1-yl, thiophen-2-yl, pyridin-3-yl, morpholino and hydroxyl, or
two adjacent groups selected from R¹, R², R⁵, R⁶ and R⁷ together with the atoms to which they are both bonded form a five member heterocyclyl ring containing two oxygen atoms and optionally substituted with two halogen atoms.

8. The compound according to claim 1, wherein R¹, R², R⁵, R⁶ and R⁷ are each independently selected from the group consisting of H, methoxy, isopropoxy, methyl, acetamido, cyano, trifluoromethoxy, difluoromethoxy, 2,2,2-trifluoroethoxy, 1,1,1-trifluoropropan-2-yloxy, trifluoromethyl, fluoro, chloro, bromo, tetrazol-5-yl, pyrazol-1-yl, thiophen-2-yl, pyridin-3-yl, morpholino and hydroxyl.

9. The compound according to claim 1, wherein R¹ is selected from the group consisting of H, methoxy, isopropoxy, methyl, acetamido, cyano, trifluoromethoxy, 2,2,2-trifluoroethoxy, 1,1,1-trifluoropropan-2-yloxy, trifluoromethyl, fluoro, chloro, pyrazol-1-yl, morpholino and hydroxyl.

10. The compound according to claim 1, wherein R² is selected from the group consisting of H, methoxy, methyl and chloro.

11. The compound according to claim 1, wherein R² is H.

12. The compound according to claim 1, wherein R⁵ is selected from the group consisting of H, methyl, trifluoromethyl and hydroxyl.

13. The compound according to claim 1, wherein R⁵ is H.

14. The compound according to claim 1, wherein R⁶ is selected from the group consisting of H, methoxy, methyl, cyano, trifluoromethoxy, difluoromethoxy, trifluoromethyl, bromo, tetrazol-5-yl, thiophen-2-yl and pyridin-3-yl.

15. The compound according to claim 1, wherein R⁷ is selected from the group consisting of H, methoxy, methyl, cyano, trifluoromethoxy, trifluoromethyl, bromo, tetrazol-5-yl and thiophen-2-yl.

16. The compound according to claim 1, wherein:
R¹ is selected from the group consisting of H, methoxy, isopropoxy, methyl, acetamido, cyano, trifluoromethoxy, 2,2,2-trifluoroethoxy, 1,1,1-trifluoropropan-2-yloxy, trifluoromethyl, fluoro, chloro, pyrazol-1-yl, morpholino and hydroxyl;
R² is selected from the group consisting of H, methoxy, methyl and chloro;
R⁵ is selected from the group consisting of H, methyl, trifluoromethyl and hydroxyl;
R⁶ is selected from the group consisting of H, methoxy, methyl, cyano, trifluoromethoxy, difluoromethoxy, trifluoromethyl, bromo, tetrazol-5-yl, thiophen-2-yl, and pyridin-3-yl; and
R⁷ is selected from the group consisting of H, methoxy, methyl, cyano, trifluoromethoxy, trifluoromethyl, bromo, tetrazol-5-yl and thiophen-2-yl.

17. The compound according to claim 1, wherein:
R¹ is selected from the group consisting of H, methoxy, isopropoxy, methyl, acetamido, cyano, trifluoromethoxy, 2,2,2-trifluoroethoxy, 1,1,1-trifluoropropan-2-yloxy, trifluoromethyl, fluoro, chloro, pyrazol-1-yl, morpholino and hydroxyl;
R² is H;
R⁵ is H;
R⁶ is selected from the group consisting of H, methoxy, methyl, cyano, trifluoromethoxy, difluoromethoxy, trifluoromethyl, bromo, tetrazol-5-yl, thiophen-2-yl and pyridin-3-yl; and
R⁷ is selected from the group consisting of H, methoxy, methyl, cyano, trifluoromethoxy, trifluoromethyl, bromo, tetrazol-5-yl and thiophen-2-yl.

18. The compound according to claim 1, wherein:
W is CR⁵;
Z is CR⁶; and
X is CR⁷.

19. The compound according to claim 1, wherein:
W is CR⁵;
Z is CR⁶; and
X is N.

20. The compound according to claim 1, wherein:
W is CR⁵;
Z is N; and
X is CR⁷.

21. The compound according to claim 1, selected from compounds of Formula (IIa) and pharmaceutically acceptable salts, solvates and hydrates thereof: 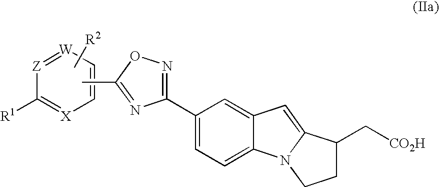
wherein:
W is N or CR⁵;
Z is N or CR⁶;
X is N or CR⁷; provided that W, Z and X are not all N, and when Z is N, then W is CR⁵ and X is CR⁷;
R¹ is selected from the group consisting of H, C₁-C₆ alkoxy, C₁-C₆ alkyl, C₁-C₆ alkylcarboxamide, cyano, C₁-C₆ haloalkoxy, C₁-C₆ haloalkyl, halogen, heteroaryl, heterocyclyl and hydroxyl;
R² is selected from the group consisting of H, C₁-C₆ alkoxy, C₁-C₆ alkyl and halogen;
R⁵ is selected from the group consisting of H, C₁-C₆ alkyl, C₁-C₆ haloalkyl and hydroxyl;
R⁶ is selected from the group consisting of H, C₁-C₆ alkoxy, C₁-C₆ alkyl, cyano, C₁-C₆ haloalkoxy, C₁-C₆ haloalkyl, halogen and heteroaryl; and
R⁷ is selected from the group consisting of H, C₁-C₆ alkoxy, C₁-C₆ alkyl, cyano, C₁-C₆ haloalkoxy, C₁-C₆ haloalkyl, halogen and heteroaryl.

22. The compound according to claim 1, selected from compounds of Formula (IIa) and pharmaceutically acceptable salts, solvates and hydrates thereof: 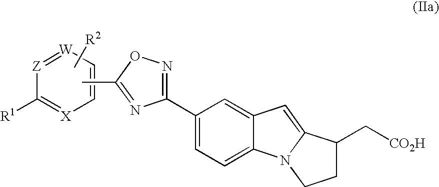
wherein:
W is N or CR⁵;
Z is N or CR⁶;
X is N or CR⁷; provided that W, Z and X are not all N, and when Z is N, then W is CR⁵ and X is CR⁷;
R¹ is selected from the group consisting of H, C₁-C₆ alkoxy, C₁-C₆ alkyl, C₁-C₆ alkylcarboxamide, cyano, C₁-C₆ haloalkoxy, C₁-C₆ haloalkyl, halogen, heteroaryl, heterocyclyl and hydroxyl;
R² is selected from the group consisting of H, methoxy, methyl and chloro;
R⁵ is selected from the group consisting of H, methyl, trifluoromethyl and hydroxyl;
R⁶ is selected from the group consisting of H, methoxy, methyl, cyano, trifluoromethoxy, difluoromethoxy, trifluoromethyl, bromo, tetrazol-5-yl, thiophen-2-yl, and pyridin-3-yl; and
R⁷ is selected from the group consisting of H, methoxy, methyl, cyano, trifluoromethoxy, trifluoromethyl, bromo, tetrazol-5-yl and thiophen-2-yl.

23. The compound according to claim 1, selected from compounds of Formula (IIa) and pharmaceutically acceptable salts, solvates and hydrates thereof: 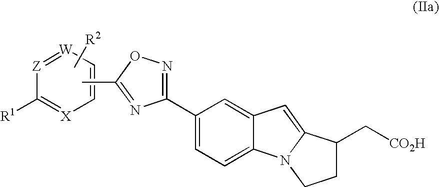
wherein:
W is N or CR⁵;
Z is N or CR⁶;
X is N or CR⁷; provided that W, Z and X are not all N, and when Z is N, then W is CR⁵ and X is CR⁷;
R¹ is selected from the group consisting of H, methoxy, isopropoxy, methyl, acetamido, cyano, trifluoromethoxy, 2,2,2-trifluoroethoxy, 1,1,1-trifluoropropan-2-yloxy, trifluoromethyl, fluoro, chloro, pyrazol-1-yl, morpholino and hydroxyl;
R² is H;
R⁵ is H;
R⁶ is selected from the group consisting of H, methoxy, methyl, cyano, trifluoromethoxy, difluoromethoxy, trifluoromethyl, bromo, tetrazol-5-yl, thiophen-2-yl and pyridin-3-yl; and
R⁷ is selected from the group consisting of H, methoxy, methyl, cyano, trifluoromethoxy, trifluoromethyl, bromo, tetrazol-5-yl and thiophen-2-yl.

24. The compound according to claim 1, selected from compounds of Formula (IIc) and pharmaceutically acceptable salts, solvates and hydrates thereof: 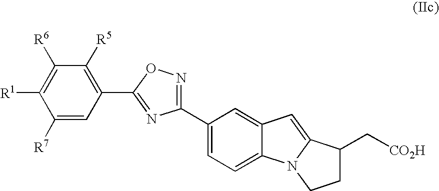
wherein:
R¹ is selected from the group consisting of H, C₁-C₆ alkoxy, C₁-C₆ haloalkoxy, halogen and hydroxyl;
R⁵ is H;
R⁶ is selected from the group consisting of C₁-C₆ alkoxy, cyano, C₁-C₆ haloalkoxy, C₁-C₆ haloalkyl, halogen and heteroaryl;
and
R⁷ is selected from the group consisting of H, C₁-C₆ alkoxy, cyano, C₁-C₆ haloalkoxy, C₁-C₆ haloalkyl, halogen and heteroaryl.

25. The compound according to claim 1, selected from compounds of Formula (IIc) and pharmaceutically acceptable salts, solvates and hydrates thereof: 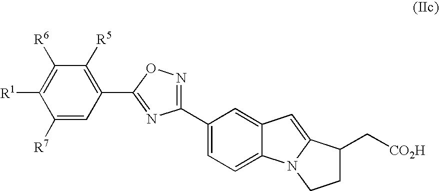
wherein:
R¹ is selected from the group consisting of H, methoxy, isopropoxy, trifluoromethoxy, 2,2,2-trifluoroethoxy, 1,1,1-trifluoropropan-2-yloxy, fluoro and hydroxyl;
R⁵ is H;
R⁶ is selected from the group consisting of cyano, trifluoromethoxy, difluoromethoxy, trifluoromethyl, bromo and pyridin-3-yl; and
R⁷ is selected from the group consisting of H, methoxy, cyano, trifluoromethoxy, trifluoromethyl and tetrazol-5-yl.

26. The compound according to claim 1, selected from compounds of Formula (IIe) and pharmaceutically acceptable salts, solvates and hydrates thereof: 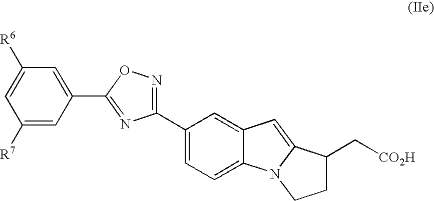
wherein:
R⁶ is selected from the group consisting of cyano, C₁-C₆ haloalkoxy, C₁-C₆ haloalkyl, halogen and heteroaryl;
and
R⁷ is selected from the group consisting of H, C₁-C₆ alkoxy, cyano, C₁-C₆ haloalkoxy, C₁-C₆ haloalkyl and heteroaryl.

27. The compound according to claim 1, selected from compounds of Formula (IIe) and pharmaceutically acceptable salts, solvates and hydrates thereof: 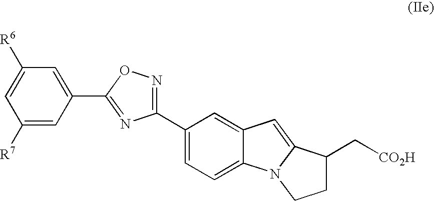
wherein:
R⁶ is selected from the group consisting of cyano, trifluoromethoxy, trifluoromethyl, bromo and pyridin-3-yl; and
R⁷ is selected from the group consisting of H, methoxy, cyano, trifluoromethoxy and trifluoromethyl.

28. The compound according to claim 1, selected from compounds of Formula (IIg) and pharmaceutically acceptable salts, solvates and hydrates thereof: 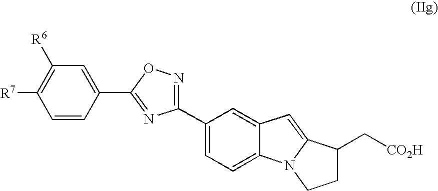
wherein:
R¹ is selected from the group consisting of H, C₁-C₆ alkoxy, C₁-C₆ haloalkoxy, halogen and hydroxyl; and
R⁶ is cyano or C₁-C₆ haloalkoxy.

29. The compound according to claim 1, selected from compounds of Formula (IIg) and pharmaceutically acceptable salts, solvates and hydrates thereof: 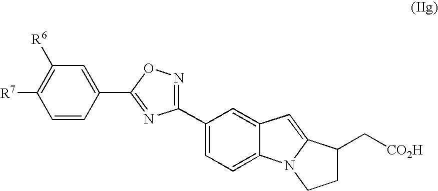
wherein:
R¹ is selected from the group consisting of H, methoxy, isopropoxy, trifluoromethoxy, 2,2,2-trifluoroethoxy, 1,1,1-trifluoropropan-2-yloxy, fluoro and hydroxyl; and
R⁶ is cyano or difluoromethoxy.

30. The compound according to claim 1, selected from compounds of Formula (IIi) and pharmaceutically acceptable salts, solvates and hydrates thereof: 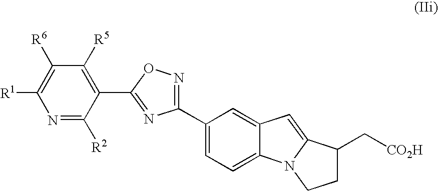
wherein:
R¹ is selected from the group consisting of H, C₁-C₆ alkoxy, C₁-C₆ alkyl, C₁-C₆ alkylcarboxamide, cyano, C₁-C₆ haloalkyl, heteroaryl and heterocyclyl;
R² is selected from the group consisting of H, C₁-C₆ alkoxy and C₁-C₆ alkyl;
R⁵ is selected from the group consisting of H, C₁-C₆ alkyl, C₁-C₆ haloalkyl and hydroxyl; and
R⁶ is selected from the group consisting of H, C₁-C₆ alkyl, and heteroaryl.

31. The compound according to claim 1, selected from compounds of Formula (IIi) and pharmaceutically acceptable salts, solvates and hydrates thereof: 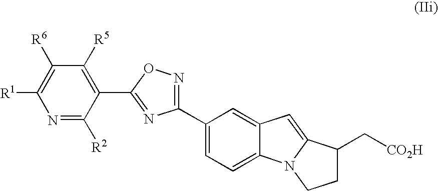
wherein:
R¹ is selected from the group consisting of H, methoxy, methyl, acetamido, cyano, trifluoromethyl, pyrazol-1-yl and morpholino;
R² is selected from the group consisting of H, methoxy and methyl;
R⁵ is selected from the group consisting of H, methyl, trifluoromethyl and hydroxyl; and
R⁶ is selected from the group consisting of H, methyl and thiophen-2-yl.

32. The compound according to claim 1, selected from the following compounds and pharmaceutically acceptable salts, solvates and hydrates thereof:
2-(7-(5-(3,5-bis(trifluoromethyl)phenyl)-1,2,4-oxadiazol-3-yl)-2,3-dihydro-1H-pyrrolo[1,2-a]indol-1-yl)acetic acid;
2-(7-(5-(3-cyano-5-(trifluoromethoxy)phenyl)-1,2,4-oxadiazol-3-yl)-2,3-dihydro-1H-pyrrolo[1,2-a]indol-1-yl)acetic acid;
2-(7-(5-(6-cyanopyridin-3-yl)-1,2,4-oxadiazol-3-yl)-2,3-dihydro-1H-pyrrolo[1,2-a]indol-1-yl)acetic acid;
2-(7-(5-(3-cyano-5-methoxyphenyl)-1,2,4-oxadiazol-3-yl)-2,3-dihydro-1H-pyrrolo[1,2-a]indol-1-yl)acetic acid;
2-(7-(5-(3-cyano-4-isopropoxyphenyl)-1,2,4-oxadiazol-3-yl)-2,3-dihydro-1H-pyrrolo[1,2-a]indol-1-yl)acetic acid;
2-(7-(5-(benzo[d][1,3 ]dioxol-5-yl)-1,2,4-oxadiazol-3-yl)-2,3-dihydro-1H-pyrrolo[1,2-a]indol-1-yl)acetic acid;
2-(7-(5-(2,6-dimethoxypyridin-3-yl)-1,2,4-oxadiazol-3-yl)-2,3-dihydro-1H-pyrrolo[1,2-a]indol-1-yl)acetic acid;
2-(7-(5-(2,2-difluorobenzo[d][1,3]dioxol-5-yl)-1,2,4-oxadiazol-3-yl)-2,3-dihydro-1H-pyrrolo[1,2-a]indol-1-yl)acetic acid;
2-(7-(5-(3-(1H-tetrazol-5-yl)-5-(trifluoromethoxy)phenyl)-1,2,4-oxadiazol-3-yl)-2,3-dihydro-1H-pyrrolo[1,2-a]indol-1-yl)acetic acid;
2-(7-(5-(4-methylpyridin-3-yl)-1,2,4-oxadiazol-3-yl)-2,3-dihydro-1H-pyrrolo[1,2-a]indol-1-yl)acetic acid;
2-(7-(5-(4-(trifluoromethyl)pyridin-3-yl)-1,2,4-oxadiazol-3-yl)-2,3-dihydro-1H-pyrrolo[1,2-a]indol-1-yl)acetic acid;
2-(7-(5-(6-methylpyridin-3-yl)-1,2,4-oxadiazol-3-yl)-2,3-dihydro-1H-pyrrolo[1,2-a]indol-1-yl)acetic acid;
2-(7-(5-(3-hydroxypyridin-2-yl)-1,2,4-oxadiazol-3-yl)-2,3-dihydro-1H-pyrrolo[1,2-a]indol-1-yl)acetic acid;
2-(7-(5-(5-methylpyridin-3-yl)-1,2,4-oxadiazol-3-yl)-2,3-dihydro-1H-pyrrolo[1,2-a]indol-1-yl)acetic acid;
2-(7-(5-(3-cyano-4-(2,2,2-trifluoroethoxy)phenyl)-1,2,4-oxadiazol-3-yl)-2,3-dihydro-1H-pyrrolo[1,2-a]indol-1-yl)acetic acid;
2-(7-(5-(4-methylpyridin-2-yl)-1,2,4-oxadiazol-3-yl)-2,3-dihydro-1H-pyrrolo[1,2-a]indol-1-yl)acetic acid;
2-(7-(5-(6-(1H-pyrazol-1-yl)pyridin-3-yl)-1,2,4-oxadiazol-3-yl)-2,3-dihydro-1H-pyrrolo[1,2-a]indol-1-yl)acetic acid;
2-(7-(5-(3-bromo-5-(trifluoromethoxy)phenyl)-1,2,4-oxadiazol-3-yl)-2,3-dihydro-1H-pyrrolo[1,2-a]indol-1-yl)acetic acid;
2-(7-(5-(3-(trifluoromethoxy)phenyl)-1,2,4-oxadiazol-3-yl)-2,3-dihydro-1H-pyrrolo[1,2-a]indol-1-yl)acetic acid;
2-(7-(5-(3-cyanophenyl)-1,2,4-oxadiazol-3-yl)-2,3-dihydro-1H-pyrrolo[1,2-a]indol-1-yl)acetic acid;
2-(7-(5-(2-methylpyridin-3-yl)-1,2,4-oxadiazol-3-yl)-2,3-dihydro-1H-pyrrolo[1,2-a]indol-1-yl)acetic acid;
2-(7-(5-(5-methylpyrazin-2-yl)-1,2,4-oxadiazol-3-yl)-2,3-dihydro-1H-pyrrolo[1,2-a]indol-1-yl)acetic acid;
2-(7-(5-(6-methylpyridin-2-yl)-1,2,4-oxadiazol-3-yl)-2,3-dihydro-1H-pyrrolo[1,2-a]indol-1-yl)acetic acid;
2-(7-(5-(2,2-difluorobenzo[d][1,3]dioxol-4-yl)-1,2,4-oxadiazol-3-yl)-2,3-dihydro-1H-pyrrolo[1,2-a]indol-1-yl)acetic acid;
2-(7-(5-(3-(difluoromethoxy)phenyl)-1,2,4-oxadiazol-3-yl)-2,3-dihydro-1H-pyrrolo[1,2-a]indol-1-yl)acetic acid;
2-(7-(5-(6-acetamidopyridin-3-yl)-1,2,4-oxadiazol-3-yl)-2,3-dihydro-1H-pyrrolo[1,2-a]indol-1-yl)acetic acid;
2-(7-(5-(6-morpholinopyridin-3-yl)-1,2,4-oxadiazol-3-yl)-2,3-dihydro-1H-pyrrolo[1,2-a]indol-1-yl)acetic acid;
2-(7-(5-(5-(thiophen-2-yl)pyridin-3-yl)-1,2,4-oxadiazol-3-yl)-2,3-dihydro-1H-pyrrolo[1,2-a]indol-1-yl)acetic acid;
2-(7-(5-(3-(pyridin-3-yl)-5-(trifluoromethoxy)phenyl)-1,2,4-oxadiazol-3-yl)-2,3-dihydro-1H-pyrrolo[1,2-a]indol-1-yl)acetic acid;
2-(7-(5-(3-cyano-4-fluorophenyl)-1,2,4-oxadiazol-3-yl)-2,3-dihydro-1H-pyrrolo[1,2-a]indol-1-yl)acetic acid;
2-(7-(5-(3-cyano-4-hydroxyphenyl)-1,2,4-oxadiazol-3-yl)-2,3-dihydro-1H-pyrrolo[1,2-a]indol-1-yl)acetic acid;
2-(7-(5-(3-cyano-4-methoxyphenyl)-1,2,4-oxadiazol-3-yl)-2,3-dihydro-1H-pyrrolo[1,2-a]indol-1-yl)acetic acid;
2-(7-(5-(3-cyano-4-(trifluoromethoxy)phenyl)-1,2,4-oxadiazol-3-yl)-2,3-dihydro-1H-pyrrolo[1,2-a]indol-1-yl)acetic acid;
2-(7-(5-(6-(trifluoromethyl)pyridin-3-yl)-1,2,4-oxadiazol-3-yl)-2,3-dihydro-1H-pyrrolo[1,2-a]indol-1-yl)acetic acid;
2-(7-(5-(2-chloro-6-methylpyridin-4-yl)-1,2,4-oxadiazol-3-yl)-2,3-dihydro-1H-pyrrolo[1,2-a]indol-1-yl)acetic acid;
2-(7-(5-(3-cyano-4-(1,1,1-trifluoropropan-2-yloxy)phenyl)-1,2,4-oxadiazol-3-yl)-2,3-dihydro-1H-pyrrolo[1,2-a]indol-1-yl)acetic acid;
2-(7-(5-(2,6-dichloropyridin-4-yl)-1,2,4-oxadiazol-3-yl)-2,3-dihydro-1H-pyrrolo[1,2-a]indol-1-yl)acetic acid; and
2-(7-(5-(5-isopropoxypyrazin-2-yl)-1,2,4-oxadiazol-3-yl)-2,3-dihydro-1H-pyrrolo[1,2-a]indol-1-yl)acetic acid.

33. The compound according to claim 1, wherein the stereochemistry of the C(1) ring carbon of said compound is R.

34. The compound according to claim 1, wherein the stereochemistry of the C(1) ring carbon of said compound is S.

35. A pharmaceutical composition comprising a compound according to claim 1 and a pharmaceutically acceptable carrier.

36. A method for treating a disease or disorder associated with the S1P1 receptor in an individual comprising administering to said individual in need thereof a therapeutically effective amount of a compound according to claim 1.

37. A method for treating a disease or disorder associated with the S1P1 receptor in an individual comprising administering to said individual in need thereof a therapeutically effective amount of a compound according to claim 1, wherein said disorder is selected from the group consisting of a disease or disorder mediated by lymphocytes, an autoimmune disease or disorder, an inflammatory disease or disorder, cancer, psoriasis, rheumatoid arthritis, Crohn's disease, transplant rejection, multiple sclerosis, systemic lupus erythematosus, ulcerative colitis, type I diabetes, sepsis, myocardial infarction, ischemic stroke and acne.

38. A method for treating a disease or disorder associated with the S1P1 receptor in an individual comprising administering to said individual in need thereof a therapeutically effective amount of a compound according to claim 1, wherein said disorder associated with the S1P1 receptor is a microbial infection or disease or a viral infection or disease.

39-45. (canceled)

46. A process for preparing a composition comprising admixing a compound according to claim 1 and a pharmaceutically acceptable carrier.
